(19) United States
(12) Patent Application Publication
(10) Pub. No.: US 2025/0195520 A1
(43) Pub. Date: Jun. 19, 2025

(54) COMBINATION THERAPY USING A PTPN11 INHIBITOR AND AN EGFR INHIBITOR

(71) Applicants: NAVIRE PHARMA, INC., Palo Alto, CA (US); BRIDGEBIO SERVICES, INC., Palo Alto, CA (US)

(72) Inventors: Pedro BELTRAN, Palo Alto, CA (US); Carl DAMBKOWSKI, Palo Alto, CA (US); Justin LIM, Palo Alto, CA (US); Anna WADE, Palo Alto, CA (US); Eli WALLACE, Palo Alto, CA (US); Yuting SUN, Palo Alto, CA (US); Nancy KOHL, Palo Alto, CA (US); Brooke MEYERS, Palo Alto, CA (US); Kerstin SINKEVICIUS, Palo Alto, CA (US); James STICE, Palo Alto, CA (US)

(21) Appl. No.: 18/696,952

(22) PCT Filed: Sep. 30, 2022
(86) PCT No.: PCT/US2022/045383

§ 371 Date: Mar. 28, 2024

Related U.S. Application Data
(60) Provisional application No. 63/250,869, filed on Sep. 30, 2021.

(51) Int. Cl. A61K 31/513; A61K 45/06; A61P 35/00

(52) U.S. Cl. CPC A61K 31/513; A61K 45/06; A61P 35/00

(57) ABSTRACT

The present disclosure provides a method of treating cancer in a subject. The method including administering to the subject: a) a therapeutically effective amount of a PTPN11 inhibitor; and b) a therapeutically effective amount of an EGFR inhibitor, wherein the PTPN11 inhibitor is represent by formula (I): (I) or a pharmaceutically acceptable salt, hydrate, solvate, stereoisomer, conformational isomer, tautomer, or a combination thereof, wherein the subscripts a and b, Y₁, Y₂, and R₁, R₂, R₃, R₄, R₅, R₆, R₇, R₈, R₉, R₁₀, R₁₁, and R₁₃ are as provided herein. In particular, the present disclosure provides a method of treating a solid tumor (e.g., an advanced non-small cell lung cancer) with a therapeutically effective amount of a compound of formula (10b) (i.e., 6-((3S,4S)-4-amino-3-methyl-2-oxa-8-azaspiro[4.5]decan-8-yl)-3-(Rₐ)-(2,3-dichlorophenyl)-2,5-dimethylpyrimidin-4(3H)-one) in combination with an EGFR inhibitor such as osimertinib.

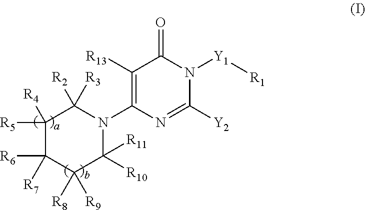

### CROSS-REFERENCES TO RELATED APPLICATIONS

This application claims priority to U.S. Provisional Application No. 63/250,869 filed Sep. 30, 2021, which is incorporated herein in its entirety for all purposes.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND

The epidermal growth factor receptor (EGFR) is a member of the ErbB family of receptors, a subfamily of four closely related receptor tyrosine kinases: EGFR (ErbB-1), HER2 (ErbB2), HER3 (ErbB3), and HER4 (ErbB4). They share a similar kinase domain structure and homology but differ in their extracellular domains and carboxy-terminal tails. The EGFRs play vital roles during development and are important regulators of cellular proliferation, survival, and migration. Mutations that lead to EGFR overexpression (known as upregulation or amplification) have been associated with a number of cancers, including adenocarcinoma of the lung (40% of cases), anal cancers, glioblastoma (50%) and epithelian tumors of the head and neck (80-100%). The identification of EGFR as an oncogene has led to the development of anticancer therapeutics directed against EGFR with EGFR inhibitors including gefitinib, erlotinib, afatinib, and icotinib for lung cancer, and cetuximab and panitumumab for colon cancer. More recently AstraZeneca has developed osimertinib, a third generation tyrosine kinase inhibitor. Additional EGFR inhibitors include vandetanib, necitumumab, brigatinib, neratinib, dacomitinib, amivantamab (JNJ-61186372), mobocertinib (TAK-788), BLU-945, varlitinib, tarloxitinib, poziotinib, and lapatinib.

Protein-tyrosine phosphatase non-receptor type 11 (PTPN11, also known as Src Homology-2 phosphatase (SHP2)) is a non-receptor protein tyrosine phosphatase encoded by the PTPN11 gene. SHP2 plays a key role in the RTK-mediated MAPK signal transduction pathway. This PTP contains two tandem Src homology-2 (SH2) domains, which function as phospho-tyrosine binding domains, a catalytic domain, and a C-terminal tail. In the basal state the protein typically exists in an inactive, self-inhibited conformation with the N-terminal SH2 domain blocking the active site. When stimulated by signal transduction mediated by cytokines and growth factor binding of phosphorylated proteins to the SH2 domains the auto-inhibition is relieved, this makes the active site available for dephosphorylation of PTPN11 substrates (MG Mohl, BG Neel, Curr. Opin. Genetics Dev. 2007, 17, 23-30. KS Grossmann, Adv. Cancer Res. 2010, 106, 53-89. W. Q. Huang et. al. Curr. Cancer Drug Targets 2014, 14, 567-588. C. Gordon et. al. Cancer Metastasis Rev. 2008, 27, 179-192.).

Germ-line and somatic mutations in PTPN11 have been reported in several human diseases resulting in gain-of-function in the catalytic activity, including Noonan Syndrome and Leopard Syndrome; as well as multiple cancers such as juvenile myelomonocytic leukemia, neuroblastoma, myelodysplastic syndrome, B cell acute lymphoblastic leukemia/lymphoma, melanoma, acute myeloid leukemia and cancers of the breast, lung and colon (MG Mohl, BG Neel, Curr. Opin. Genetics Dev. 2007, 17, 23-30). Recent studies have demonstrated that single PTPN11 mutations are able to induce Noonan syndrome, JMML-like myeloproliferative disease and acute leukemia in mice. These mutations disrupt the auto-inhibition between the N—SH2 domains and the catalytic site allowing constitutive access of substrates to the catalytic site of the enzyme (E. Darian et al, Proteins, 2011, 79, 1573-1588. Z-H Yu et al, JBC, 2013, 288, 10472, W Qiu et al BMC Struct. Biol. 2014, 14, 10).

PTPN11 is widely expressed in most tissues and plays a regulatory role in various cell signaling events that are important for a diversity of cell functions that includes proliferation, differentiation, cell cycle maintenance, epithelial-mesenchymal transition (EMT), mitogenic activation, metabolic control, transcription regulation, and cell migration, through multiple signaling pathways including the Ras-MAPK, the JAK-STAT or the PI3K-AKT pathways (Tajan, M. et. al. Eur. J. Medical Genetics, 2015, 58, 509-525. Prahallad, A. et. al. Cell Reports, 2015, 12, 1978-1985).

Additionally there is growing evidence that PTPN11/SHP2 is implicated in immune evasion during tumorigenesis, and hence a SHP2 inhibitor could stimulate the immune response in cancer patients (Cancer Res. 2015 Feb. 1; 75 (3): 508-18. T Yokosuka T, J Exp Med. 2012, 209 (6), 1201. S Amarnath Sci Transl Med. 2011, 3, 111ra120. T Okazaki, PNAS 2001, 98:24, 13866-71).

As cancers may have or develop resistance to EGFR inhibitors, there remains a need for effective and safe therapeutic agents, including agents that may be used in combination, to treat cancers.

### BRIEF SUMMARY

The present disclosure provides methods of treating diseases and disorders (e.g., cancers) by administering both a PTPN11 inhibitor (e.g., a compound represented by formula (I), such as formula (10b), as described herein) and an EGFR inhibitor.

In a first aspect, the present disclosure provides a method of treating cancer in a subject, the method including administering to the subject:
a) a therapeutically effective amount of a PTPN11 inhibitor; and
b) a therapeutically effective amount of an EGFR inhibitor,
   wherein the PTPN11 inhibitor is represent by formula (I):

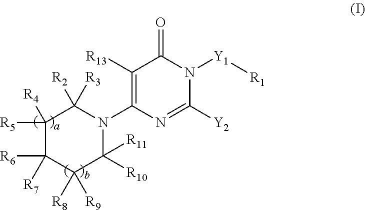

or a pharmaceutically acceptable salt, hydrate, solvate, stereoisomer, conformational isomer, tautomer, or a combination thereof, wherein the subscripts a and b, Y₁, Y₂, and R₁, R₂, R₃, R₄, R₅, R₆, R₇, R₈, R₉, R₁₀, R₁₁, and R₁₃ are as provided herein.

In a second aspect, the present disclosure provides a method of treating a solid tumor (e.g., an advanced non-small cell lung cancer) in a subject, the method including administering to a subject in need thereof:
a) a therapeutically effective amount of a compound represented by formula (10b):

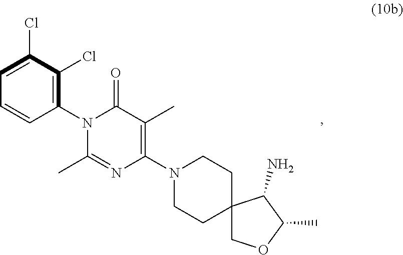

or a pharmaceutically acceptable salt, hydrate, solvate, stereoisomer, conformational isomer, tautomer, or a combination thereof; and
b) a therapeutically effective amount of an EGFR inhibitor.

In a third aspect, the present disclosure provides a pharmaceutical composition for treating cancer in a subject, the composition including:
a) a therapeutically effective amount of a PTPN11 inhibitor; and
b) a therapeutically effective amount of an EGFR inhibitor,
   together with a pharmaceutically acceptable carrier or excipient,
      wherein the PTPN11 inhibitor is represent by formula (I) as defined and described herein.

In a fourth aspect, the present disclosure provides a kit for treating cancer in a subject, the kit including:
a) a therapeutically effective amount of a PTPN11 inhibitor; and
b) a therapeutically effective amount of an EGFR inhibitor,
   together with instruction for effective administration,
      wherein the PTPN11 inhibitor is represent by formula (I) as defined and described herein.

### BRIEF DESCRIPTION OF THE DRAWINGS

FIGS. 1A-1B show treatment with the combination of osimertinib and formula (10b) synergistically suppresses the proliferation of HCC4006 human tumor cell lines in vitro. FIG. 1A shows a representative crystal violet stained plate at the end of the experiment; FIG. 1B shows the average Bliss score at indicated concentrations of osimertinib and formula (10b). The colors and numbers in the heatmap indicate whether the combination effect is synergistic (Bliss score>0), additive (Bliss score-0), or antagonistic (Bliss score<0).

FIGS. 2A-2B show single agent activity of osimertinib in HCC4006 parental and HCC4006-OsiR tumor cell lines in vitro. FIG. 2A shows in vitro 3-day proliferation assay was used. FIG. 2B shows in vitro 14-day clonogenic assay was used. Dotted line denotes where 50% inhibition of relative cell number.

FIGS. 3A-3B show treatment with the combination of osimertinib and formula (10b) synergistically suppresses the proliferation of HCC4006-OsiR human tumor cell lines in vitro. FIG. 3A shows a representative crystal violet stained plate at the end of the experiment. FIG. 3B shows average Bliss score at indicated concentrations of osimertinib and formula (10b). The colors and numbers in the heatmap indicate whether the combination effect is synergistic (Bliss score>0), additive (Bliss score=0), or antagonistic (Bliss score<0).

FIG. 4 shows treatment with formula (10b) as a single agent or in combination with osimertinib reduces the transcript levels of MAPK pathway signature gene DUSP6 in HCC4006-OsiR cells in vitro.

FIGS. 5A-5B show treatment with formula (10b) as a single agent or in combination with osimertinib suppresses the growth of the HCC827 subcutaneous tumors in vivo, at tolerated doses. FIG. 5A shows tumor volume monitored bi-weekly by caliper measurement; and FIG. 5B shows body weights recorded daily.

FIGS. 6A-6B shows tumor volume monitored bi-weekly by caliper following treatment with formula (10b) as a single agent or in combination with osimertinib suppresses the growth of the HCC827-ER1 tumors (FIG. 6A) and NCI-H1875 (C797S+) tumors (FIG. 6B), at tolerated doses.

FIG. 7 shows treatment with formula (10b) as a single agent or in combination with osimertinib suppresses DUSP6 mRNA levels in the HCC827-ER1 tumors. ** represents p<0.01. The dotted horizontal line denotes 50% suppression of DUSP6 mRNA levels.

FIG. 8 shows treatment with formula (10b) as a single agent or in combination with osimertinib suppresses the MPAS-plus signature in the HCC827-ER1 tumors.

FIG. 9 shows a flowchart for a trial conducted using the BOIN Design. Abbreviations: BOIN-Bayesian optimal interval design; DLT=dose limiting toxicity; MTD=maximum tolerated dose. Note: λe=19.7% and λd=29.8%. In practice, with 6 patients/cohort, if the DLT rate is ≤1/6 then escalate the dose, if the DLT rate is ≥2/6 then de-escalate the dose.

### DETAILED DESCRIPTION

### I. General

The present disclosure provides a combination therapy method of treating a disease or disorder (e.g., cancer, such as a solid tumor) in a subject. The method includes administering to the subject a) a therapeutically effective amount of a PTPN11 inhibitor; and b) a therapeutically effective amount of an EGFR inhibitor, wherein the PTPN11 inhibitor is represented by formula (I) as defined and described herein (e.g., a compound represented by formula (10b)). The EGFR inhibitor may at least partially inhibit EGFR kinase. The EGFR inhibitor may be a selective EGFR inhibitor. Also provided are a pharmaceutical composition thereof and a kit thereof for treating a disease or disorder (e.g., cancer) in a subject.

### II. Definitions

As used herein, the terms below have the meanings indicated.

“Comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

“About,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

“Acyl,” as used herein, alone or in combination, refers to a carbonyl attached to an alkenyl, alkyl, aryl, cycloalkyl, heteroaryl, heterocycle, or any other moiety were the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

“Alkenyl,” as used herein, alone or in combination, refers to a straight-chain or branched-chain hydrocarbon radical having one or more double bonds and containing from 2 to 20 carbon atoms. In certain embodiments, said alkenyl will comprise from 2 to 6 carbon atoms. The term “alkenylene” refers to a carbon-carbon double bond system attached at two or more positions such as ethenylene [(—CH═CH—), (—C::C—)]. Examples of suitable alkenyl radicals include ethenyl, propenyl, 2-methylpropenyl, 1,4-butadienyl and the like. Unless otherwise specified, the term “alkenyl” may include “alkenylene” groups.

“Alkynyl” refers to either a straight chain or branched hydrocarbon having at least 2 carbon atoms and at least one triple bond and having the number of carbon atom indicated (i.e., C₂₋₆ means to two to six carbons). Alkynyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Examples of alkynyl groups include, but are not limited to, acetylenyl, propynyl, 1-butynyl, 2-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, and 1,3,5-hexatriynyl.

“Alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

“Alkyl,” as used herein, alone or in combination, refers to a straight-chain or branched-chain alkyl radical containing from 1 to 20 carbon atoms. In certain embodiments, said alkyl will comprise from 1 to 10 carbon atoms. In further embodiments, said alkyl will comprise from 1 to 8 carbon atoms. Alkyl groups are unsubstituted or substituted as defined herein. Examples of alkyl radicals include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

“Alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like.

“Alkylthio,” as used herein, alone or in combination, refers to an alkyl thioether (R—S—) radical wherein the term alkyl is as defined above and wherein the sulfur may be singly or doubly oxidized. Examples of suitable alkyl thioether radicals include methylthio, ethylthio, n-propylthio, isopropylthio, n-butylthio, iso-butylthio, sec-butylthio, tert-butylthio, methanesulfonyl, ethanesulfinyl, and the like.

“Amido” and “carbamoyl,” as used herein, alone or in combination, refer to an amino group as described below attached to the parent molecular moiety through a carbonyl group, or vice versa. The “amido” group as used herein incudes a “C-amido” and “N-amido” groups. The term “C-amido” as used herein, alone or in combination, refers to a —C(O)N(RR′) group with R and R′ as defined herein or as defined by the specifically enumerated “R” groups designated. In some embodiments, the “amido” group includes —C(O) NH₂, C₁₋₄alkylamido, and di(C₁₋₄alkyl)amido. The term “C₁₋₄alkylamido”, as used herein, refers to —C(O) NH(C₁₋₄alkyl), wherein C₁₋₄alkyl is as defined herein. The term “N-amido” as used herein, alone or in combination, refers to a RC(O)N(R′)— group, with R and R′ as defined herein or as defined by the specifically enumerated “R” groups designated. The term “acylamino” as used herein, alone or in combination, embraces an acyl group attached to the parent moiety through an amino group. An example of an “acylamino” group is acetylamino (CH₃C(O)NH—).

“Amino,” as used herein, alone or in combination, refers to —NRR′, wherein R and R′ are independently selected from hydrogen, alkyl, acyl, heteroalkyl, aryl, cycloalkyl, heteroaryl, and heterocycloalkyl, any of which may themselves be unsubstituted or substituted. Additionally, R and R′ may combine to form heterocycloalkyl, either of which is unsubstituted or substituted.

“Aryl,” as used herein, alone or in combination, means a carbocyclic aromatic system containing one, two or three rings wherein such polycyclic ring systems are fused together. The term “aryl” embraces aromatic groups such as phenyl, naphthyl, anthracenyl, and phenanthryl.

“Arylalkenyl” or “aralkenyl,” as used herein, alone or in combination, refers to an aryl group attached to the parent molecular moiety through an alkenyl group.

“Arylalkoxy” or “aralkoxy,” as used herein, alone or in combination, refers to an aryl group attached to the parent molecular moiety through an alkoxy group.

“Arylalkyl” or “aralkyl,” as used herein, alone or in combination, refers to an aryl group attached to the parent molecular moiety through an alkyl group.

“Aryloxy,” as used herein, alone or in combination, refers to an aryl group attached to the parent molecular moiety through an oxy.

“Carbamate,” as used herein, alone or in combination, refers to an ester of carbamic acid (—NHCOO—) which may be attached to the parent molecular moiety from either the nitrogen or acid end, and which is unsubstituted or substituted as defined herein.

“O-carbamyl” as used herein, alone or in combination, refers to a —OC(O)NRR′, group—with R and R′ as defined herein.

“N-carbamyl” as used herein, alone or in combination, refers to a ROC(O)NR′— group, with R and R′ as defined herein.

“Carbonyl,” as used herein, when alone includes formyl [—C(O)H] and in combination is a —C(O)— group.

“Carboxyl” or “carboxy,” as used herein, refers to —C(O) OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

“Cyano,” as used herein, alone or in combination, refers to —CN.

“Cycloalkyl,” or, alternatively, “carbocycle,” as used herein, alone or in combination, refers to a saturated or partially saturated monocyclic, bicyclic or tricyclic alkyl group wherein each cyclic moiety contains from 3 to 12 carbon atom ring members and which may optionally be a benzo fused ring system which is unsubstituted or substituted as defined herein. The term “cycloalkenyl” refers to a cycloalkyl group having one or two double bonds. In certain embodiments, said cycloalkyl (or cycloalkenyl) will comprise from 5 to 7 carbon atoms. Examples of such groups include cyclopropyl, cyclobutyl, cyclopentyl, cyclohexyl, cycloheptyl, cyclobutenyl, cyclopentenyl, cyclohexenyl, tetrahydronapthyl, indanyl, octahydronaphthyl, 2,3-dihydro-1H-indenyl, adamantyl and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1,1,1] pentane, camphor, adamantane, and bicyclo[3,2,1]octane.

“Ester,” as used herein, alone or in combination, refers to a carboxy group bridging two moieties linked at carbon atoms.

“Ether,” as used herein, alone or in combination, refers to an oxy group bridging two moieties linked at carbon atoms.

“Halo,” or “halogen,” as used herein, alone or in combination, refers to fluorine, chlorine, bromine, or iodine.

“Haloalkoxy,” as used herein, alone or in combination, refers to a haloalkyl group attached to the parent molecular moiety through an oxygen atom.

“Haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro, or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

“Heteroalkyl,” as used herein, alone or in combination, refers to a stable straight or branched chain, or a combination thereof, fully saturated or containing from 1 to 3 degrees of unsaturation, consisting of the stated number of carbon atoms and from one to three heteroatoms selected from N, O, and S, and wherein the N and S atoms may optionally be oxidized and the N heteroatom may optionally be quaternized. The heteroatom(s) may be placed at any interior position of the heteroalkyl group. Up to two heteroatoms may be consecutive, such as, for example, —CH₂—NH—OCH₃.

“Heteroaryl,” as used herein, alone or in combination, refers to a 3 to 15 membered unsaturated heteromonocyclic ring, or a fused monocyclic, bicyclic, or tricyclic ring system in which at least one of the fused rings is aromatic, which contains at least one atom selected from N, O, and S. In certain embodiments, said heteroaryl will comprise from 1 to 4 heteroatoms as ring members. In further embodiments, said heteroaryl will comprise from 1 to 2 heteroatoms as ring members. In certain embodiments, said heteroaryl will comprise from 5 to 7 atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

“Heterocycloalkyl” and, interchangeably, “heterocycle,” as used herein, alone or in combination, each refer to a saturated, partially unsaturated, or fully unsaturated (but nonaromatic) monocyclic, bicyclic, or tricyclic heterocyclic group containing at least one heteroatom as a ring member, wherein each said heteroatom may be independently selected from nitrogen, oxygen, and sulfur. In certain embodiments, said heterocycloalkyl will comprise from 1 to 4 heteroatoms as ring members. In further embodiments, said heterocycloalkyl will comprise from 1 to 2 heteroatoms as ring members. In certain embodiments, said heterocycloalkyl will comprise from 3 to 8 ring members in each ring. In further embodiments, said heterocycloalkyl will comprise from 3 to 7 ring members in each ring. In yet further embodiments, said heterocycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include aziridinyl, azetidinyl, 1,3-benzodioxolyl, dihydroisoindolyl, dihydroisoquinolinyl, dihydrocinnolinyl, dihydrobenzodioxinyl, dihydro [1,3]oxazolo[4,5-b]pyridinyl, benzothiazolyl, dihydroindolyl, dihydropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups are unsubstituted or substituted unless specifically prohibited.

“Hydrazinyl” as used herein, alone or in combination, refers to two amino groups joined by a single bond, i.e., —N—N—.

“Hydroxy,” as used herein, alone or in combination, refers to —OH.

“Hydroxyalkyl,” as used herein, alone or in combination, refers to a hydroxy group attached to the parent molecular moiety through an alkyl group.

“Iminohydroxy,” as used herein, alone or in combination, refers to ═N(OH) and ═N—O—.

“Lower amino,” as used herein, alone or in combination, refers to —NRR′, wherein R and R′ are independently selected from hydrogen and lower alkyl, either of which is unsubstituted or substituted.

“Mercaptyl” as used herein, alone or in combination, refers to an RS— group, where R is as defined herein.

“Nitro,” as used herein, alone or in combination, refers to —NO₂.

“Oxy” or “oxa,” as used herein, alone or in combination, refer to —O—.

“Oxo,” as used herein, alone or in combination, refers to ═O.

“Perhaloalkoxy” refers to an alkoxy group where all of the hydrogen atoms are replaced by halogen atoms.

“Perhaloalkyl” as used herein, alone or in combination, refers to an alkyl group where all of the hydrogen atoms are replaced by halogen atoms.

“Ring,” or equivalently, “cycle,” as used herein, in reference to a chemical structure or portion thereof, means a group in which every atom is a member of a common cyclic structure. A ring can be saturated or unsaturated, including aromatic, unless otherwise provided, and may have between 3 and 9 members. If the ring is a heterocycle, it may contain between 1 and 4 heteroatoms or heteroatom-comprising groups selected from B, N, O, S, C(O), S(O)m. Unless specifically prohibited, a ring is unsubstituted or substituted.

“Sulfonate,” “sulfonic acid,” and “sulfonic,” as used herein, alone or in combination, refer to the —SO₃H group and its anion as the sulfonic acid is used in salt formation.

“Sulfanyl,” as used herein, alone or in combination, refers to —S—.

“Sulfinyl,” as used herein, alone or in combination, refers to —S(O)—.

“Sulfonyl,” as used herein, alone or in combination, refers to —S(O)₂—.

“N-sulfonamido” refers to a RS(═O)₂NR′— group with R and R′ as defined herein.

“S-sulfonamido” refers to a —S(═O)₂NRR′, group, with R and R′ as defined herein.

“Thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

“Thiol,” as used herein, alone or in combination, refers to an —SH group.

“Thiocarbonyl,” as used herein, when alone includes thioformyl —C(S)H and in combination is a —C(S)— group.

“N-thiocarbamyl” refers to an ROC(S)NR′— group, with R and R′ as defined herein.

“O-thiocarbamyl” refers to a —OC(S)NRR′, group with R and R′ as defined herein.

“Thiocyanato” refers to a —CNS group.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

“Bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

“Salt” refers to acid or base salts of the compounds of the present disclosure. Illustrative examples of pharmaceutically acceptable acid addition salts are mineral acid (hydrochloric acid, hydrobromic acid, phosphoric acid, and the like) salts and organic acid (acetic acid, propionic acid, glutamic acid, citric acid and the like) salts. Examples of pharmaceutically acceptable base addition salts include sodium, potassium, calcium, ammonium, organic amino, or magnesium salt, or a similar salt. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

“Solvate” refers to a compound provided herein or a salt thereof, that further includes a stoichiometric or non-stoichiometric amount of solvent bound by non-covalent intermolecular forces.

“Hydrate” refers to a compound that is complexed to a water molecule. The compounds of the present disclosure can be complexed with ½ water molecule or from 1 to 10 water molecules.

Asymmetric centers exist in the compounds disclosed herein. These centers are designated by the symbols “R” or “S,” depending on the configuration of substituents around the chiral carbon atom. It should be understood that the present disclosure encompasses all stereochemical isomeric forms, including diastereomeric, enantiomeric, and epimeric forms, as well as d-isomers and l-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method. Starting compounds of particular stereochemistry are either commercially available or can be made and resolved by various techniques. Additionally, the compounds disclosed herein may exist as geometric isomers. The present disclosure includes all cis, trans, syn, anti, entgegen (E), and zusammen (Z) isomers as well as the appropriate mixtures thereof. Additionally, compounds may exist as tautomers; all tautomeric isomers are provided by this disclosure. Additionally, the compounds disclosed herein can exist in unsolvated as well as solvated forms with pharmaceutically acceptable solvents such as water, ethanol, and the like. In general, the solvated forms are considered equivalent to the unsolvated forms.

“Tautomer”, as use herein, alone or in combination, refers to one of two or more isomers that rapidly interconvert. Generally, this interconversion is sufficiently fast so that an individual tautomer is not isolated in the absence of another tautomer. The ratio of the amount of tautomers can be dependent on solvent composition, ionic strength, and pH, as well as other solution parameters. The ratio of the amount of tautomers can be different in a particular solution and in the microenvironment of a biomolecular binding site in said solution. Examples of tautomers include keto/enol, enamine/imine, and lactam/lactim tautomers. Additional examples of tautomers also include 2-hydroxypyridine/2 (1H)-pyridone and 2-aminopyridine/2 (1H)-iminopyridone tautomers.

Conformational isomers exist in the compounds disclosed herein. When R₁ is aryl or heteroaryl in the formula:

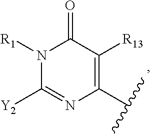

the aryl or heteroaryl group can orient in different conformations in relation to the pyrimidinone moiety, as represented by:

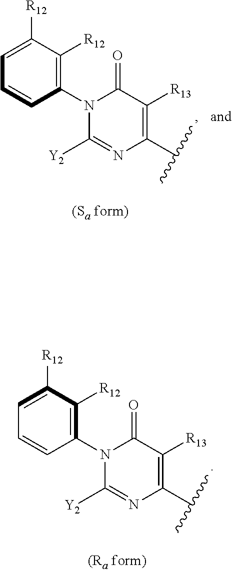

These forms are designated by the symbols “Sₐ” or “Rₐ”, depending on the conformation of the aryl or heteroaryl group in relation to the pyrimidinone moiety. Examples of “Sₐ” and “Rₐ” forms can be found in Examples 1-20 of International Patent Application No. PCT/US2019/045903, which is incorporated herein in its entirety for all purposes. The compound of formula (10b) is substantially in a “Rₐ” form.

“Pharmaceutically acceptable” refers to those compounds (salts, hydrates, solvates, stereoisomers, conformational isomers, tautomers, etc.) which are suitable for use in contact with the tissues of patients without undue toxicity, irritation, and allergic response, are commensurate with a reasonable benefit/risk ratio, and are effective for their intended use. The compounds disclosed herein can exist as pharmaceutically acceptable salts, as defined and described herein.

“Combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

“PTPN11 inhibitor” is used herein to refer to a compound that exhibits an IC₅₀ with respect to PTPN11 activity of no more than about 100 micromolar (μM) and more typically not more than about 50 μM, as measured in the PTPN11 assay described generally in International Patent Application No. PCT/US2019/045903 (e.g., the enzymatic activity of recombinant human PTPN11 proteins of Example 21). “IC₅₀” is that concentration of inhibitor which reduces the activity of an enzyme (e.g., PTPN11) to half-maximal level. In certain embodiments, compounds exhibit disclosed in PCT/US2019/045903 exhibit an IC₅₀ of no more than about 10 μM for inhibition of PTPN11; in further embodiments, compounds exhibit an IC₅₀ of no more than about 1 μM for inhibition of PTPN11; in yet further embodiments, compounds exhibit an IC₅₀ of not more than about 200 nM for inhibition of PTPN11; in yet further embodiments, compounds exhibit an IC₅₀ of not more than about 100 nM for inhibition of PTPN11; and in yet further embodiments, compounds exhibit an IC₅₀ of not more than about 50 nM for inhibition of PTPN11, as measured in the PTPN11 assay described therein. In certain embodiments, the compound of formula (2b) exhibits an IC₅₀ of no more than 150 nM for inhibition of PTPN11 (e.g., a PTPN11-E76K mutant enzyme). In certain embodiments, the compound of formula (10b) exhibits an IC₅₀ of no more than 50 nM for inhibition of PTPN11 (e.g., a PTPN11-E76K mutant enzyme).

“Therapeutically effective amount” refers to an amount of a compound or of a pharmaceutical composition useful for treating or ameliorating an identified disease or condition, or for exhibiting a detectable therapeutic or inhibitory effect. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Treat”, “treating”, and “treatment” refer to any indicia of success in the treatment or amelioration of an injury, pathology, or condition, including any objective or subjective parameter such as abatement; remission; diminishing of symptoms or making the injury, pathology or condition more tolerable to the patient; slowing in the rate of degeneration or decline; making the final point of degeneration less debilitating; and/or improving a patient's physical or mental well-being. The treatment or amelioration of symptoms can be based on objective or subjective parameters; including the results of a physical examination, neuropsychiatric exams, and/or a psychiatric evaluation.

“Administering” refers to therapeutic provision of the compound or a form thereof to a subject, such as by oral administration or intravenous administration.

“Patient” or “subject” refers to a living organism suffering from or prone to a disease or condition that can be treated by administration of a pharmaceutical composition as provided herein. Non-limiting examples include humans, non-human primates (e.g., monkeys), goats, pigs, sheep, cows, deer, horses, bovines, rats, mice, rabbits, hamsters, guinea pigs, cats, dogs, and other non-mammalian animals. In some embodiments, the subject is human. In some embodiments, a subject is an adult (e.g., at least 18 years of age).

“Composition,” as used herein, is intended to encompass a product comprising the specified ingredients in the specified amounts, as well as any product, which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

“Pharmaceutically acceptable excipient” refers to a substance that aids the administration of an active agent to and absorption by a subject. Pharmaceutical excipients useful in the present disclosure include, but are not limited to, binders, fillers, disintegrants, lubricants, coatings, sweeteners, flavors and colors. Other pharmaceutical excipients can also be useful in the present disclosure.

“Tablet” refers to solid pharmaceutical formulations with and without a coating. The term “tablet” also refers to tablets having one, two, three or even more layers, wherein each of the before mentioned types of tablets may be without or with one or more coatings. In some embodiments, tablets of the present disclosure can be prepared by roller compaction or other suitable means known in the art. The term “tablet” also comprises mini, melt, chewable, effervescent, and orally disintegrating tablets. Tablets include the compound of formula (I) or (10b) and one or more pharmaceutical excipients (e.g., fillers, binders, glidants, disintegrants, surfactants, binders, lubricants, and the like). Optionally, a coating agent can be also included. For the purposes of calculating percent weight of the tablet formulation, the amount of coating agent is not included in the calculation. That is, the percent weights reported herein are of the uncoated tablet.

Unless specifically indicated otherwise, the content of the compound of formula (I) (10b) in, e.g., a tablet formulation is calculated based on the normalized weight of the compound of formula (I) or (10b) on a salt-free and anhydrous basis. That is, the salt and/or water content in the compound of formula (I) or (10b) is not included in the calculation.

“EGFR inhibitor” as used herein refers to a compound which targets, decreases, or inhibits the synthesis or biological activity of epidermal growth factor receptor (EGFR). The EGFR inhibitor may at least partially inhibit EGFR. The EGFR inhibitor may be a selective EGFR inhibitor. In those cases, the selective EGFR inhibitor may have high potency for EGFR, along with low affinity for other related kinases. Examples of EGFR inhibitors include gefitinib, erlotinib, afatinib, icotinib, cetuximab, panitumumab, osimertinib, vandetanib, necitumumab, brigatinib, neratinib, dacomitinib, amivantamab (JNJ-61186372), mobocertinib (TAK-788), BLU-945, varlitinib, tarloxitinib, poziotinib, and lapatinib.

“EGFR-positive cancer” refers to a cancer with the EGFR gene rearranged, mutated, or amplified.

“A cancer resistant to an EGFR inhibitor” and “a cancer that is an EGFR-positive cancer resistant to an EGFR inhibitor” refer to a cancer or tumor that either fails to respond favorably to treatment with a prior EGFR inhibitor, or alternatively, recurs or relapses after responding favorably to an EGFR inhibitor.

“Jointly therapeutically effective amount” as used herein means the amount at which the therapeutic agents, when given separately (in a chronologically staggered manner, especially a sequence-specific manner) to a warm-blooded animal, especially to a human to be treated, show an (additive, but preferably synergistic) interaction (joint therapeutic effect). Whether this is the case can be determined inter alia by following the blood levels, showing that both compounds are present in the blood of the human to be treated at least during certain time intervals.

“Synergistic effect” as used herein refers to an effect of at least two therapeutic agents: a PTPN11 inhibitor as defined herein; and an EGFR inhibitor as defined herein, which is greater than the simple addition of the effects of each drug administered by themselves. The effect can be, for example, slowing the symptomatic progression of a proliferative disease, such as cancer, particularly lung cancer, or symptoms thereof. Analogously, a “synergistically effective amount” refers to the amount needed to obtain a synergistic effect.

“A,” “an,” or “a (n)”, when used in reference to a group of substituents or “substituent group” herein, mean at least one. For example, where a compound is substituted with “an” alkyl or aryl, the compound is substituted with at least one alkyl and/or at least one aryl, wherein each alkyl and/or aryl is optionally different. In another example, where a compound is substituted with “a” substituent group, the compound is substituted with at least one substituent group, wherein each substituent group is optionally different.

### III. Combination Therapy

In a first aspect, the present disclosure provides a method of treating cancer in a subject. The method includes administering to the subject:
a) a therapeutically effective amount of a PTPN11 inhibitor; and
b) a therapeutically effective amount of an EGFR inhibitor
   wherein the PTPN11 inhibitor is represent by formula (I):

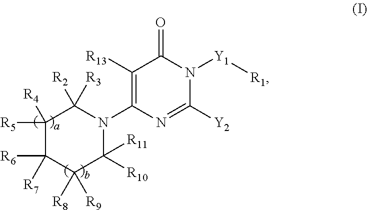

or a pharmaceutically acceptable salt, hydrate, solvate, stereoisomer, conformational isomer, tautomer, or a combination thereof,
wherein:
subscript a is 0 or 1;
subscript b is 0 or 1;
Y₁ is a direct bond or CR₁₇R₁₈;
Y₂ is selected from the group consisting of C₁₋₄alkyl, amino, C₁₋₄alkylC(O)O—, C₁₋₄alkylamino and C₁₋₄aminoalkyl;
R₁ is selected from the group consisting of C₆₋₁₀aryl, C₃₋₈cycloalkyl, C₃₋₈cycloalkenyl, and a 5-10 membered heteroaryl group having 1 to 4 heteroatoms or groups as ring vertices independently selected from N, C(O), O, and S; said aryl or heteroaryl of R₁ is unsubstituted or substituted with 1 to 5 R₁₂ groups independently selected from the group consisting of halo, hydroxy, amino, C₁₋₄alkylamino, di(C₁₋₄alkyl)amino, cyano, C₁₋₄alkyl, C₁₋₄alkoxy, C₁₋₄hydroxyalkyl, C₁₋₄haloalkyl, C₁₋₄aminoalkyl, C₃₋₈cycloalkyl, C₃₋₈cycloalkenyl, NR₁₅C(O) R₁₄, NR₁₅C(O)OR₁₄, NR₁₄C(O)NR₁₅R₁₆, NR₁₅S(O) R₁₄, NR₁₅S(O)₂R₁₄, C(O)NR₁₅R₁₆, S(O)NR₁₅R₁₆, S(O)₂NR₁₅R₁₆, C(O) R₁₄, C(O)OR₁₄, OR₁₄, SR₁₄, S(O) R₁₄, and S(O)₂R₁₄;
R₂, R₃, R₁₀, and R₁₁ are each independently selected from the group consisting of hydrogen, C₁₋₄alkyl, and C₃₋₈cycloalkyl;
R₄, R₅, R₈, and R₉ are each independently selected from the group consisting of hydrogen, cyano, C₁₋₄alkyl, C₁₋₄alkoxy, amino, hydroxy, C₃₋₈cycloalkyl, halo, and C₁₋₄alkylamino;
R₆ is selected from the group consisting of amino, C₁₋₄aminoalkyl, and C₁₋₄alkylamino;
R₇ is selected from the group consisting of hydrogen, amido, cyano, halo, and hydroxy, or is selected from the group consisting of C₁₋₄alkyl, C₁₋₄hydroxyalkyl, C₃₋₆cycloalkyl, phenyl, and 5- or 6-membered heteroaryl, any of which is unsubstituted or substituted with 1 to 5 groups independently selected from the group consisting of amino, halo, hydroxy, cyano, C₁₋₄alkyl, C₁₋₄alkoxy, C₁₋₄haloalkyl, C₁₋₄haloalkoxy, C₁₋₄alkylamino, and C₁₋₄aminoalkyl;
or R₆ and R₇ together with the carbon atom to which they are both attached form a 3- to 7-membered saturated or unsaturated ring, having 0 to 3 heteroatoms or groups as ring vertices independently selected from N, C(O), O, and S(O) m; subscript m is 0, 1, or 2; and said saturated or unsaturated ring formed by R₆ and R₇ is unsubstituted or substituted with 1 to 3 groups independently selected from the group consisting of amino, halo, hydroxy, cyano, C₁₋₄alkyl, C₁₋₄alkoxy, C₁₋₄haloalkyl, C₁₋₄haloalkoxy, C₁₋₄alkylamino and C₁₋₄aminoalkyl;
any two groups of R₂, R₃, R₄, R₅, R₇, R₈, R₉, R₁₀ and R₁₁ can form a 5 to 6 membered ring, having 0 to 2 heteroatoms as ring vertices elected from N, O and S;
any two groups of R₂, R₄, R₆, R₈ and R₁₀ can form a direct bond, or a 1 or 2 atom carbon bridge;
R₁₃ is selected from the group consisting of hydrogen, halo, cyano, C₁₋₆alkyl, C₁₋₆haloalkyl, C₁₋₆hydroxyalkyl, C₁₋₆ dihydroxyalkyl, —NH—NHR₁₉, —NHR₁₉, —OR₁₉, —NHC(O) R₁₉, —NHC(O) NHR₁₉, —NHS(O)₂NHR₁₉, —NHS(O)₂R₁₉, —C(O)OR₁₉, —C(O)NR₁₉R₂₀, —C(O) NH(CH₂)_{q}OH, —C(O) NH(CH₂)_{q}R₂₁, —C(O) R₂₁, —NH₂, —OH, —S(O)₂NR₁₉R₂₀, C₃₋₈cycloalkyl, aryl, heterocyclyl having 1-5 heteroatoms as ring vertices selected from N, O, S and P, and heteroaryl having 1-5 heteroatoms as ring vertices selected from N, O, S and P; subscript q is an integer of from 0 to 6; and each of aryl, heteroaryl, heterocyclyl and cycloalkyl of R₁₃ is unsubstituted or substituted with 1 to 3 groups independently selected from the group consisting of C₁₋₄alkyl, —OH, —NH₂, —OR₂₁, halo, cyano, and oxo;
R₁₄, R₁₅ and R₁₆ are each independently selected from the group consisting of hydrogen, C₁₋₄alkyl, C₃₋₈cycloalkyl, C₆₋₁₀aryl and 5-10 membered heteroaryl, any of which is unsubstituted or substituted with one or more groups independently selected from the group consisting of amido, amino, halo, hydroxy, cyano, C₁₋₄alkyl, C₁₋₄alkoxy, C₁₋₄haloalkyl, C₁₋₄haloalkoxy, C₁₋₄alkylamino and C₁₋₄aminoalkyl; R₁₇ and R₁₈ are each independently selected from the group consisting of hydrogen, C₁₋₄alkyl, and CF₃;
R₁₉ and R₂₀ are each independently selected from the group consisting of hydrogen, C₁₋₆alkyl, C₁₋₆haloalkyl, C₁₋₆hydroxyalkyl, C₂₋₆alkenyl, C₂₋₆alkynyl and C₃₋₆cycloalkyl; and
each R₂₁ is independently selected from the group consisting of hydrogen, —OH, C₁₋₆ alkyl, C₁₋₆haloalkyl, C₁₋₆hydroxyalkyl, C₂₋₆alkenyl, C₂₋₆alkynyl and C₃₋₆cycloalkyl.
   III-1: PTPN11 Inhibitors and/or EGFR Inhibitors

The PTPN11 inhibitor represented by formula (I) is further described according to Section IV. Compounds. In some embodiments, the PTPN11 inhibitor of formula (I) is any one of embodiments as described in Section IV. Compounds.

In some embodiments, the PTPN11 inhibitor is represented by formula (2b):

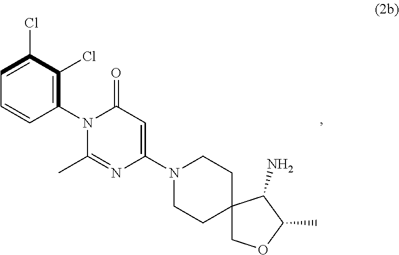

having the name of 6-((3S,4S)-4-amino-3-methyl-2-oxa-8-azaspiro[4.5]decan-8-yl)-3-(Rₐ)-(2,3-dichlorophenyl)-2-methylpyrimidin-4 (3H)-one.

In some embodiments, the PTPN11 inhibitor is represented by formula (10b):

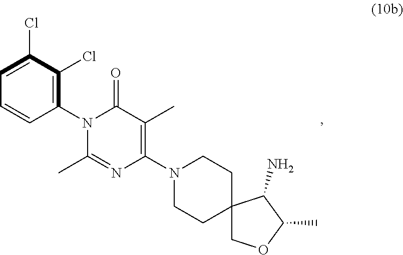

having the name of 6-((3S,4S)-4-amino-3-methyl-2-oxa-8-azaspiro[4.5]decan-8-yl)-3-(Rₐ)-(2,3-dichlorophenyl)-2,5-dimethylpyrimidin-4 (3H)-one.

The compound of any one of formula (I), formula (2b), and formula (10b) can be in a pharmaceutically acceptable salt form or in a neutral form, each of which is optionally in a solvate or a hydrate form.

In some embodiments, the compound of any one of formula (I), formula (2b), and formula (10b) is in a pharmaceutically acceptable salt form. In some embodiments, a pharmaceutically acceptable acid addition salt of the compound of formula (10b) is represented by formula (10b-HX):

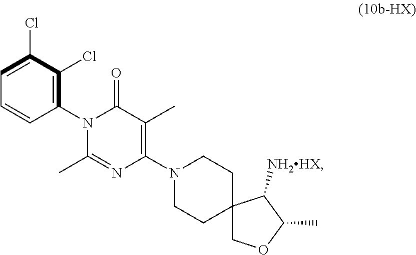

wherein HX is a pharmaceutically acceptable acid addition.

Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like.

In some embodiments, the compound of any one of formula (I), formula (2b), and formula (10b) is in a neutral form. In some embodiments, the compound of formula (10b) is in a neutral form.

In some embodiments, the compound of formula (10b) has a substantially moiety of 6-((3S,4S)-4-amino-3-methyl-2-oxa-8-azaspiro[4.5]decan-8-yl) with stereochemistry as shown in formula (10b):

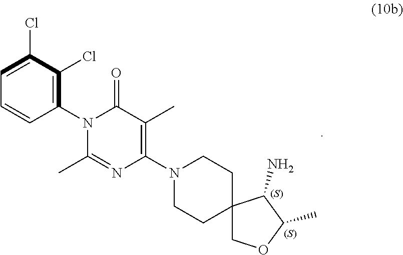

In some embodiments, the compound of formula (10b) is substantially in a Rₐ conformation as shown in formula (10b):

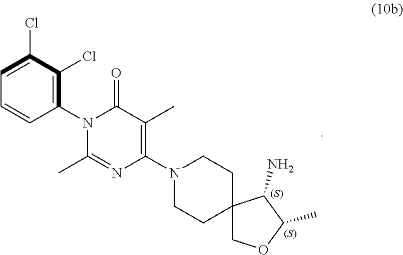

In some embodiments, the compound of formula (10b) is represented by the formula:

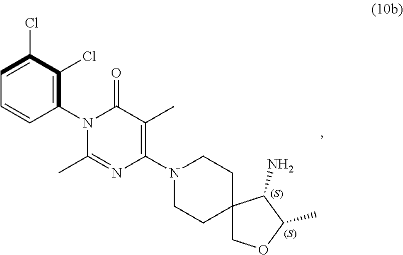

having the name of 6-((3S,4S)-4-amino-3-methyl-2-oxa-8-azaspiro[4.5]decan-8-yl)-3-(Rₐ)-(2,3-dichlorophenyl)-2,5-dimethylpyrimidin-4 (3H)-one.

In some embodiments, the compound of formula (10b) includes one or more corresponding enantiomer, diastereomers, and/or conformational isomers, as represented by formulae, respectively:

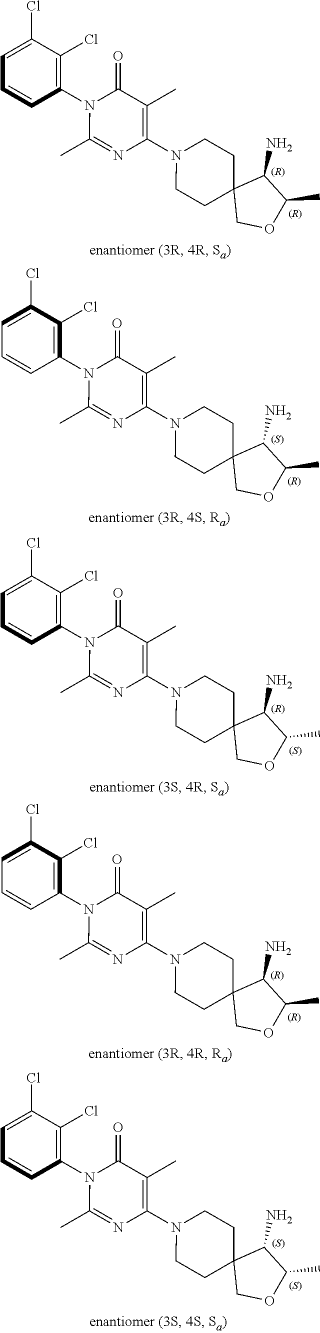

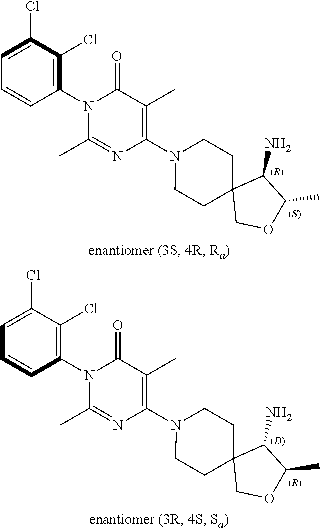

In some embodiments, the compound of formula (10b) has a purity of at least about 95 area % determined by a chiral high-performance liquid chromatography (HPLC). In some embodiments, the compound of formula (10b) has a purity of from about 95 area % to about 99 area %, from about 96 area % to about 99 area %, from about 97 area % to about 99 area %, or from about 98 area % to about 99 area %, determined by a chiral high-performance liquid chromatography (HPLC). In some embodiments, the compound of formula (10b) has a purity of from about 98 area % to about 99 area %.

In some embodiments, the compound of formula (10b) includes one or more corresponding enantiomer, diastereomers, and/or conformational isomers, as represented by the formulae above; and a total of the one or more isomers is no more than about 5 area % determined by a chiral high-performance liquid chromatography (HPLC).

In some embodiments, the corresponding enantiomer, diastereomers, and/or conformational isomers of the compound of formula (10b) are present in the compound of formula (10b) meet acceptance criteria as follows: enantiomer (3R, 4R, Sₐ)≤0.5 area %; diastereomer (3R, 4S, Rₐ)≤1.2 area %; diastereomer (3S, 4R, Sₐ)≤0.5 area %; diastereomer (3R, 4R, Rₐ)≤0.5 area %; diastereomer (3S, 4S, Sₐ)≤0.5 area %; diastereomer (3S, 4R, Rₐ)≤0.5 area %; and diastereomer (3R, 4S, Sₐ)≤0.5 area %, each of which is determined by a chiral high-performance liquid chromatography (HPLC). In some embodiments, the compound of formula (10b) has a purity of at least about 95 area %, wherein enantiomer (3R, 4R, Sₐ)<0.5 area %; diastereomer (3R, 4S, Rₐ)<1.2 area %; diastereomer (3S, 4R, Sₐ)<0.5 area %; diastereomer (3R, 4R, Rₐ)<0.5 area %; diastereomer (3S, 4S, Sₐ)<0.5 area %; diastereomer (3S, 4R, Rₐ)<0.5 area %; and diastereomer (3R, 4S, Sₐ)<0.5 area %, each of which is determined by a chiral high-performance liquid chromatography (HPLC). In some embodiments, the compound of formula (10b) has a purity of from about 95 area % to about 99 area %, from about 96 area % to about 99 area %, from about 97 area % to about 99 area %, or from about 98 area % to about 99 area %, wherein enantiomer (3R, 4R, Sₐ)<0.5 area %; diastereomer (3R, 4S, Rₐ)<1.2 area %; diastereomer (3S, 4R, Sₐ)<0.5 area %; diastereomer (3R, 4R, Rₐ)<0.5 area %; diastereomer (3S, 4S, Sₐ)<0.5 area %; diastereomer (3S, 4R, Rₐ)<0.5 area %; and diastereomer (3R, 4S, Sₐ)<0.5 area %, each of which is determined by a chiral high-performance liquid chromatography (HPLC). In some embodiments, the compound of formula (10b) has a purity of from about 98 area % to about 99 area %, wherein enantiomer (3R, 4R, Sₐ) is not detected; diastereomer (3R, 4S, Rₐ) is about 0.86 area %; diastereomer (3S, 4R, Sₐ) is not detected; diastereomer (3R, 4R, Rₐ) is about 0.07 area %; diastereomer (3S, 4S, Sₐ) is not detected; diastereomer (3S, 4R, Rₐ) is not detected; and diastereomer (3R, 4S, Sₐ) is not detected, each of which is determined by a chiral high-performance liquid chromatography (HPLC).

In some embodiments, the compound of any one of formula (I), formula (2b), formula (10b), and formula (10b-HX) is in a solvate and/or a hydrate form.

The EGFR inhibitor can be an inhibitor described for use in the treatment of a cancer. In some embodiments, the EGFR inhibitor at least partially inhibits EGFR kinase. In some embodiments, the EGFR inhibitor is an EGFR/HER2 dual inhibitor. In some embodiments, the EGFR inhibitor is a selective EGFR inhibitor.

In some embodiments, the EGFR inhibitor is erlotinib, cetuximab, panitumumab, vandetanib, afatinib, gefitinib, osimertinib, necitumumab, brigatinib, neratinib, dacomitinib, amivantamab (JNJ-61186372), mobocertinib (TAK-788), BLU-945, varlitinib, tarloxitinib, poziotinib, or lapatinib. In some embodiments, the EGFR inhibitor is osimertinib. In some embodiments, the EGFR inhibitor is erlotinib.

In some embodiments, the PTPN11 inhibitor is represented by formula (2b); and the EGFR inhibitor is erlotinib, cetuximab, panitumumab, vandetanib, afatinib, gefitinib, osimertinib, necitumumab, brigatinib, neratinib, dacomitinib, amivantamab (JNJ-61186372), mobocertinib (TAK-788), BLU-945, varlitinib, tarloxitinib, poziotinib, or lapatinib. In some embodiments, the PTPN11 inhibitor is represented by formula (2b); and the EGFR inhibitor is osimertinib. In some embodiments, the PTPN11 inhibitor is represented by formula (2b); and the EGFR inhibitor is erlotinib.

In some embodiments, the PTPN11 inhibitor is represented by formula (10b); and the EGFR inhibitor is erlotinib, cetuximab, panitumumab, vandetanib, afatinib, gefitinib, osimertinib, necitumumab, brigatinib, neratinib, dacomitinib, amivantamab (JNJ-61186372), mobocertinib (TAK-788), BLU-945, varlitinib, tarloxitinib, poziotinib, or lapatinib. In some embodiments, the PTPN11 inhibitor is represented by formula (10b); and the EGFR inhibitor is osimertinib. In some embodiments, the PTPN11 inhibitor is represented by formula (10b); and the EGFR inhibitor is erlotinib.

### III-2. Cancer/Solid Tumor

The cancer can be any cancer that responds to the treatment of a PTPN11 inhibitor and/or an EGFR inhibitor. In some embodiments, the cancer is an EGFR-positive cancer (e.g., a cancer characterized by a mutation in EGFR).

In some embodiments, the cancer is characterized by a mutation in EGFR, as described herein. In some embodiments, the cancer is characterized by an EGFR mutation including an EGFR exon 19 deletion, exon 20 insertion, L858X mutation, T790X mutation, C797X mutation, G719X mutation, L861X mutation, S768X mutation, E709X mutation, or any combination thereof. In some embodiments, the cancer is characterized by an EGFR mutation including an EGFR exon 19 deletion, and/or exon 20 insertion. In some embodiments, the cancer is characterized by an EGFR exon 19 deletion. In some embodiments, the cancer is characterized by an EGFR exon 20 insertion.

The cancer can be characterized by a solid tumor or a liquid tumor. In some embodiments, the cancer includes a solid tumor. In some embodiments, the cancer includes a liquid tumor.

In some embodiments, the cancer is biliary tract cancer, brain cancer, breast cancer, cervical cancer, colorectal cancer, esophageal cancer, gastric cancer, head and neck squamous cell carcinoma (HNSCC), lung cancer, pancreatic cancer, thyroid cancer, or a combination thereof. In some embodiments, the cancer is non-small cell lung cancer (NSCLC). In some embodiments, the cancer is NSCLC characterized by an EGFR mutation, such as an EGFR exon 19 deletion, and/or exon 20 insertion. In some embodiments, an EGFR mutation includes an EGFR exon 19 deletion, exon 20 insertion, L858X mutation, T790X mutation, C797X mutation, G719X mutation, L861X mutation, S768X mutation, E709X mutation, or any combination thereof. In some embodiments, the cancer is NSCLC characterized by an EGFR exon 19 deletion. In some embodiments, the cancer is NSCLC characterized by an EGFR exon 20 insertion. In some embodiments, the cancer is NSCLC that is not characterized by a mutation in KRAS or anaplastic lymphoma kinase (ALK).

In some embodiments, the cancer is an EGFR-positive cancer (e.g., a cancer characterized by a mutation in EGFR). In some embodiments, the cancer is an advanced or metastatic EGFR-positive solid tumor (e.g., biliary tract cancer, brain cancer, breast cancer, cervical cancer, colorectal cancer, esophageal cancer, gastric cancer, head and neck squamous cell carcinoma (HNSCC), lung cancer, pancreatic cancer, thyroid cancer, or a combination thereof). In some embodiments, the cancer is an advanced or metastatic EGFR-positive non-small cell lung cancer (NSCLC). In some embodiments, the cancer is an advanced or metastatic EGFR-positive solid tumor, provided that the solid tumor is other than non-small cell lung cancer (NSCLC).

The cancer can also be any cancer that is resistant to the treatment of an EGFR inhibitor (e.g., a selective EGFR inhibitor or an EGFR/HER2 dual inhibitor). In some embodiments, the cancer is resistant to an EGFR inhibitor. In some embodiments, the cancer is characterized by intrinsic and/or acquired resistance to an EGFR inhibitor. In some embodiments, the cancer is an EGFR-positive cancer resistant to an EGFR inhibitor. In some embodiments, the cancer is an EGFR-positive cancer characterized by intrinsic and/or acquired resistance to an EGFR inhibitor. In some embodiments, the cancer is characterized by EGFR-dependent and/or EGFR-independent resistance to an EGFR inhibitor.

In some embodiments, the cancer is resistant to an EGFR inhibitor selected from the group consisting of erlotinib, cetuximab, panitumumab, vandetanib, afatinib, gefitinib, osimertinib, necitumumab, brigatinib, neratinib, dacomitinib, amivantamab (JNJ-61186372), mobocertinib (TAK-788), BLU-945, varlitinib, tarloxitinib, poziotinib, and lapatinib. In some embodiments, the cancer is resistant to osimertinib. In some embodiments, the cancer is resistant to erlotinib. In some embodiments, the cancer is an EGFR-positive cancer resistant to an EGFR inhibitor selected from the group consisting of erlotinib, cetuximab, panitumumab, vandetanib, afatinib, gefitinib, osimertinib, necitumumab, brigatinib, neratinib, dacomitinib, amivantamab (JNJ-61186372), mobocertinib (TAK-788), BLU-945, varlitinib, tarloxitinib, poziotinib, and lapatinib. In some embodiments, the cancer is an EGFR-positive cancer resistant to osimertinib. In some embodiments, the cancer is an EGFR-positive cancer resistant to erlotinib.

In some embodiments, the cancer is characterized by intrinsic and/or acquired resistance to another therapy such as a KRAS modulator, platinum-based therapy, or taxane therapy. In some embodiments, the cancer is characterized by intrinsic and/or acquired resistance to a KRAS inhibitor, such as a KRAS G12C inhibitor (e.g., sotorasib or adagrasib). In some embodiments, the cancer is characterized by intrinsic and/or acquired resistance to a platinum-based therapy. In some embodiments, the cancer is characterized by intrinsic and/or acquired resistance to a taxane therapy.

In some embodiments, the cancer is characterized by intrinsic and/or acquired resistance to an EGFR inhibitor. In some embodiments, the cancer is characterized by intrinsic and/or acquired resistance to an EGFR inhibitor selected from the group consisting of erlotinib, cetuximab, panitumumab, vandetanib, afatinib, gefitinib, osimertinib, necitumumab, brigatinib, neratinib, dacomitinib, amivantamab (JNJ-61186372), mobocertinib (TAK-788), BLU-945, varlitinib, tarloxitinib, poziotinib, and lapatinib. In some embodiments, the cancer is characterized by intrinsic and/or acquired resistance to osimertinib. In some embodiments, the cancer is characterized by intrinsic and/or acquired resistance to erlotinib.

The solid tumor can be any solid tumor that responds to the treatment of a PTPN11 inhibitor and an EGFR inhibitor (e.g., osimertinib or erlotinib). In some embodiments, the solid tumor is a tumor with one or more genes in EGFR rearranged, mutated, or amplified. In some embodiments, the solid tumor is a tumor with one or more genes in EGFR rearranged, mutated, or amplified, provided that the tumor is not characterized by one or more additional activating mutations in BRAF V600X, PTPN11 (SHP2), or KRAS Q61X.

In some embodiments, the solid tumor is an advanced or metastatic non-small cell lung cancer (NSCLC) caused by a mutation in EGFR. In some embodiments, the solid tumor is an advanced or metastatic non-small cell lung cancer (NSCLC) caused by a mutation in EGFR, provided that the tumor is not characterized by one or more additional activating mutations in BRAF V600X, PTPN11 (SHP2), or KRAS Q61X. In some embodiments, the solid tumor is an EGFR-positive solid tumor. In some embodiments, the solid tumor is an advanced or metastatic EGFR-positive non-small cell lung cancer (NSCLC).

The solid tumor can also be any tumor that is resistant to the treatment of an EGFR inhibitor (e.g., erlotinib, cetuximab, panitumumab, vandetanib, afatinib, gefitinib, osimertinib, necitumumab, brigatinib, neratinib, dacomitinib, amivantamab (JNJ-61186372), mobocertinib (TAK-788), BLU-945, varlitinib, tarloxitinib, poziotinib, or lapatinib). In some embodiments, the solid tumor is resistant to an EGFR inhibitor (e.g., as described herein). In some embodiments, the solid tumor is characterized by intrinsic and/or acquired resistance to an EGFR inhibitor (e.g., as described herein). In some embodiments, the solid tumor is an EGFR solid tumor resistant to an EGFR inhibitor (e.g., as described herein). In some embodiments, the solid tumor is an EGFR-positive solid tumor characterized by intrinsic and/or acquired resistance to an EGFR inhibitor (e.g., as described herein). In some embodiments, the solid tumor is resistant to the treatment of an EGFR inhibitor selected from the group consisting of erlotinib, cetuximab, panitumumab, vandetanib, afatinib, gefitinib, osimertinib, necitumumab, brigatinib, neratinib, dacomitinib, amivantamab (JNJ-61186372), mobocertinib (TAK-788), BLU-945, varlitinib, tarloxitinib, poziotinib, and lapatinib. In some embodiments, the solid tumor is resistant to osimertinib. In some embodiments, the solid tumor is resistant to erlotinib. In some embodiments, the solid tumor is an EGFR-positive solid tumor resistant to the treatment of an EGFR inhibitor selected from the group consisting of erlotinib, cetuximab, panitumumab, vandetanib, afatinib, gefitinib, osimertinib, necitumumab, brigatinib, neratinib, dacomitinib, amivantamab (JNJ-61186372), mobocertinib (TAK-788), BLU-945, varlitinib, tarloxitinib, poziotinib, and lapatinib. In some embodiments, the solid tumor is an EGFR-positive solid tumor resistant to osimertinib. In some embodiments, the solid tumor is an EGFR-positive solid tumor resistant to erlotinib.

In any one of embodiments, a standard of care or curative therapy is unavailable for treating the cancer or solid tumor, as described herein.

### III-3: Subject

In some embodiments, the subject is human. In some embodiments, the subject is under the care of a medical practitioner, such as a physician. In some embodiments, the subject has been diagnosed with the cancer. In some embodiments, the subject has relapsed. In some embodiments, the subject has previously entered remission. In some embodiments, the subject has previously undergone, is undergoing, or will undergo a monotherapy course of treatment. In some embodiments, the subject has previously undergone, is undergoing, or will undergo radiation therapy. In some embodiments, the subject has previously undergone, is undergoing, or will undergo immunotherapy. In some embodiments, the subject has previously undergone, is undergoing, or will undergo chemotherapy. In some embodiments, the subject has previously undergone, is undergoing, or will undergo a platinum-based chemotherapy. In some embodiments, the subject has previously undergone, is undergoing, or will undergo a therapeutic regimen comprising administration of a KRAS modulator (e.g., KRAS inhibitor, such as a KRAS G12C inhibitor). In some embodiments, the subject has previously undergone, is undergoing, or will undergo a therapeutic regimen comprising administration of an EGFR inhibitor. In some embodiments, the subject has previously undergone, is undergoing, or will undergo a therapeutic regimen comprising administration of a PTPN11 inhibitor. In some embodiments, the subject has previously undergone, is undergoing, or will undergo a therapeutic regimen comprising administration of an anti-PD-1/PD-L1 inhibitor (e.g., checkpoint inhibitor).

The subject can have an advanced (e.g., primary, metastatic, or recurrent) solid tumor with a RAS mutation, such as a mutation in KRAS, NRAS, and/or HRAS. In some embodiments, the subject has a mutation in KRAS, NRAS, and/or HRAS. In some embodiments, the subject has a KRAS mutation such as a KRAS G12C, G12D, G12V, G12R, G12A, G12S, G13C, G13D, G13V, G13R, G13A, G13S, or Q61X mutation. In some embodiments, the subject has a KRAS, NRAS, and/or HRAS mutation other than a Q61X mutation. In some embodiments, the subject has a KRAS, NRAS, and/or HRAS mutation as assessed by molecular diagnostic using an appropriate clinically validated and/or FDA approved test and with no available standard of care or curative therapies. In some embodiments, the subject has a KRAS, NRAS, and/or HRAS mutation mutation, as assessed by molecular diagnostic using an appropriate clinically validated and/or FDA approved test within at least two (2) years prior to the admission to the treatment as described herein. In some embodiments, the subject has a KRAS, NRAS, and/or HRAS mutation other than a Q61X mutation as assessed by molecular diagnostic using an appropriate clinically validated and/or FDA approved test and with no available standard of care or curative therapies.

In some embodiments, the subject has cancer characterized by one or more mutations in EGFR, as described herein. In some embodiments, subject has cancer characterized by an EGFR mutation including an EGFR exon 19 deletion, exon 20 insertion, L858X mutation, T790X mutation, C797X mutation, G719X mutation, L861X mutation, S768X mutation, E709X mutation, or any combination thereof. In some embodiments, subject has cancer characterized by an EGFR mutation including an EGFR exon 19 deletion, and/or exon 20 insertion. In some embodiments, subject has cancer characterized by an EGFR exon 19 deletion. In some embodiments, subject has cancer characterized by an EGFR exon 20 insertion.

In some embodiments, the subject has a mutation in EGFR (e.g., the subject has a cancer characterized by a mutation in EGFR), as described herein. In some embodiments, subject has an EGFR mutation including an EGFR exon 19 deletion, exon 20 insertion, L858X mutation, T790X mutation, C797X mutation, G719X mutation, L861X mutation, S768X mutation, E709X mutation, or any combination thereof. In some embodiments, subject has an EGFR mutation including an EGFR exon 19 deletion, and/or exon 20 insertion. In some embodiments, subject has an EGFR exon 19 deletion. In some embodiments, subject has an EGFR exon 20 insertion.

In some embodiments, the subject has a mutation in PTPN11. In some embodiments, the subject has a mutation in PTPN11 including an E76K mutation. In some embodiments, the subject does not have a mutation in PTPN11, such as an E76K mutation.

In some embodiments, the subject has the solid tumor progressed or recurred on or after at least one prior line of a systemic therapy including a platinum-based doublet chemotherapy and/or an anti-PD-1/PD-L1 therapy, each of which is given in monotherapy or both of which are given in combination therapy.

In some embodiments, the subject has a measurable disease according to response evaluation criteria in solid tumors (RECIST). In some embodiments, treatment of the subject with the compound of formula (I) or (10b) and the EGFR inhibitor causes a measurable change in disease state according to RECIST.

In some embodiments, prior to administration of the compound of formula (I) or (10b) and the EGFR inhibitor (e.g., as described herein), the subject has not undergone treatment with any chemotherapy or other investigational therapy such as hormonal (including corticosteroids), biological, or targeted agents for ≥3 weeks; or the subject is at least 5 half-lives from hormonal (including corticosteroids), biological, or targeted agents, whichever is longer at the time of treatment initiation.

In some embodiments, prior to administration of the compound of formula (I) or (10b) and the EGFR inhibitor (e.g., as described herein), the subject is not previously treated with a cancer therapy comprising a chemotherapy, a hormone therapy, an immunotherapy or biological therapy, a targeted therapy, or a combination thereof.

In some embodiments, the subject that has been or is on a cancer therapy including a chemotherapy, a hormone therapy, an immunotherapy or biological therapy, a targeted therapy, or a combination thereof is treated with the compound of formula (I) or (10b) and the EGFR inhibitor (e.g., as described herein) if the subject discontinues such cancer therapy (e.g., a chemotherapy, a hormone therapy, an immunotherapy or biological therapy, a targeted therapy, or a combination thereof) for a period of at least about three weeks or five (5) half-lives of an agent used in the cancer therapy, whichever is longer prior to initiation of the treatment with the the compound of formula (I) or (10b) and the EGFR inhibitor (e.g., as described herein).

In some embodiments, the subject does not have one or more additional activating mutations in PTPN11 (SHP2), MEK, or RAS (e.g., NRAS, HRAS, KRAS) (such as a Q61 mutation). In some embodiments, the subject does not have one or more additional activating mutations in BRAF V600X, PTPN11 (SHP2), and/or KRAS (e.g., a KRAS Q61X mutation). In some embodiments, the subject does not have a tumor harboring one or more additional activating mutations in BRAF V600X, PTPN11 (SHP2), and/or KRAS (e.g., a KRAS Q61X mutation).

In some embodiments, prior to administration of the compound of formula (I) or (10b) and the EGFR inhibitor (e.g., as described herein), the subject has not previously taken or is not taking one or more of strong or moderate inducers or inhibitors of CYP3A4 and/or P-gp inducers or inhibitors (including herbal supplements).

In some embodiments, the subject has taken or is taking one or more of strong or moderate inducers or inhibitors of CYP3A4 and/or P-gp inducers or inhibitors (including herbal supplements) is treated with the compound of formula (I) or (10b) and the EGFR inhibitor (e.g., as described herein) if the subject discontinues such treatment for a period of at least about five (5) half-lives prior to initiation of the treatment with the compound of formula (I) or (10b) and the EGFR inhibitor (e.g., as described herein) and during the treatment period of the the compound of formula (I) or (10b) and the EGFR inhibitor (e.g., as described herein).

In some embodiments, prior to administration of the compound of formula (I) or (10b) and the EGFR inhibitor (e.g., as described herein), the subject has not previously taken or is not taking a drug that is a known substrate of P-gp, BCRP, OATP1B1, OATP1B3, MATE1, and/or MATE2-K transporters.

In some embodiments, the subject has taken or is taking a drug that is a known substrate of P-gp, BCRP, OATP1B1, OATP1B3, MATE1, and/or MATE2-K transporters is treated with the compound of formula (I) or (10b) if the subject discontinues such treatment prior to initiation of the treatment with the compound of formula (I) or (10b) and the EGFR inhibitor (e.g., as described herein) and during the treatment period of the compound of formula (I) or (10b) and the EGFR inhibitor (e.g., as described herein).

In some embodiments, the subject has not previously participated in an interventional clinical study within a period of at least about four (4) weeks or five (5) half-lives of an agent used in the interventional clinical study, whichever is shorter prior to initiation of the treatment with the compound of formula (I) or (10b) in combination with the EGFR inhibitor.

In some embodiments, the subject has not previously received a radiotherapy or a proton therapy including i) a limited field of radiation for palliation within a period of about one (1) week, or ii) a radiation to more than about 30% of bone marrow or a wide field of radiation within a period of about four (4) weeks, prior to initiation of the treatment with the compound of formula (I) or (10b) in combination with the EGFR inhibitor.

In some embodiments, the subject has not taken or is not taking a) one or more of strong or moderate inducers or inhibitors of CYP3A4 and/or P-gp inducers or inhibitors (including herbal supplements or food products containing grapefruit juice, star fruit, or Seville oranges) within a period of about 14 days or five (5) half-lives, whichever is longer prior to initiation of the treatment with the compound of formula (I) or (10b) in combination with the EGFR inhibitor; b) a drug that is a known substrate of CYP3A4, P-gp, multidrug and toxin extrusion protein (MATE)1, and/or MATE2-K transporters within a period of about 14 days or five (5) half-lives, whichever is longer prior to initiation of the treatment with the compound of formula (I) or (10b) in combination with the EGFR inhibitor; and/or one or more acid reducing agents, such as proton pump inhibitors (PPIs) or H2 receptor antagonists within a period of about 14 days or five (5) half-lives, whichever is longer prior to initiation of the treatment with the compound of formula (I) or (10b) in combination with the EGFR inhibitor.

In some embodiments, the subject does not have inadequate organ functions including adequate hematological, renal, hepatic, and coagulating functions, as defined below:

### Hematological

a. Absolute neutrophil count<1,500/μL;
b. Platelets<100,000/μL; and
c. Hemoglobin<9 g/dL without transfusion for ≤2 weeks or erythropoiesis-stimulating agents (e.g., Epo, Procrit) for ≤6 weeks.

### Renal

d. Serum creatinine>1.5×ULN, unless creatinine clearance≥40 mL/min (measured or calculated using the Cockcroft-Gault formula)

### Hepatic

e. Serum total bilirubin≥1.5× institutional upper limit of normal (ULN) or ≥3.0× institutional ULN if the patient has a diagnosis of Gilbert syndrome or hemolytic anemia as confirmed by the investigator; and
f. Aspartate aminotransferase/serum glutamic-oxaloacetic transaminase (AST/SGOT) and/or alanine aminotransferase/serum glutamic-pyruvic transaminase (ALT/SGPT)>2.5×ULN.

### Coagulation

g. International normalized ratio (INR) or prothrombin time (PT)>1.5×ULN unless the patient is receiving anticoagulant therapy and as long as PT or activated partial thromboplastin time (aPTT) is within the therapeutic range of intended use of anticoagulants; and
h. Activated partial thromboplastin time>1.5×ULN unless the patient is receiving anticoagulant therapy and as long as PT or aPTT is within the therapeutic range of intended use of anticoagulants.

In some embodiments, the subject does not have active hepatitis B infection, hepatitis C infection, or human immunodeficiency virus (HIV) infection with measurable viral load.

In some embodiments, the subject does not have has a life-threatening illness, medical condition, an active uncontrolled infection, or an organ system dysfunction (e.g., ascites, coagulopathy, or encephalopathy).

In some embodiments, the subject does not have one or more cardiac-related diseases or findings:
a) History of significant cardiovascular disease (e.g., cerebrovascular accident, myocardial infarction or unstable angina), within the last 6 months before starting the treatment;
b) Clinically significant cardiac disease, including New York Heart Association Class II or higher heart failure;
c) History of left ventricular ejection fraction (LVEF)<50% within the previous 12 months before starting the treatment;
d) Resting corrected QT interval (QTc)>470 msec, derived as the averaged from three electrocardiograms (ECGs), using the ECG machines provided; and/or
e) Any clinically significant abnormalities in rhythm, conduction, or morphology of resting ECG (e.g., third degree heart block, Mobitz Type II heart block, ventricular arrhythmias, uncontrolled atrial fibrillation).

In some embodiments, the subject does not have an additional malignancy that is progressing or requires an active treatment, wherein the additional malignancy includes basal cell carcinoma of the skin, squamous cell carcinoma of the skin that has undergone potentially curative therapy or in situ cervical cancer.

In some embodiments, the subject has not been diagnosed of an additional invasive malignancy within the previous 3 years, provided that the additional invasive malignancy is other than curatively treated non-melanomatous skin cancer, superficial urothelial carcinoma, in situ cervical cancer, or any other curatively treated malignancy that is not expected to require treatment for recurrence during the course of the treatment with the compound of formula (I) or (10b) in combination with the EGFR inhibitor.

In some embodiments, the subject does not have one or more untreated brain metastases from non-brain tumors.

In some embodiments, the subject does not have a primary central nervous system (CNS) tumor, an active CNS metastasis, and/or a carcinomatous meningitis. In some embodiments, the subject does not have a primary central nervous system (CNS) tumor. In some embodiments, the subject does not have an active CNS metastasis, and/or a carcinomatous meningitis.

In some embodiments, the subject has brain metastases and is treated with the compound of formula (I) or (10b) and the EGFR inhibitor (e.g., as described herein), if i) the brain metastases are stable (without evidence of progression by imaging for at least four weeks prior to administration of the compound of formula (I) or (10b) and the EGFR inhibitor (e.g., as described herein) and any neurologic symptoms have returned to baseline); ii) the subject has no evidence of new or enlarging brain metastases; and iii) the subject is not using steroids and/or anti-seizures medications for at least 7 days prior to prior to administration of the the compound of formula (I) or (10b) and the EGFR inhibitor (e.g., as described herein), provided that the subject does not have carcinomatous meningitis.

In some embodiments, the subject who has had brain metastases resected or have received radiation therapy ending at least 4 weeks prior to the initiation of the treatment (e.g., Cycle 1, Day 1) with the compound of formula (I) or (10b) in combination with the EGFR inhibitor is eligible, provided that the subject meets all of the following criteria prior to the initiation of the treatment: a) residual neurological symptoms related to the CNS treatment Grade≤2; b) on a stable or decreasing dose of ≤10 mg daily prednisone (or equivalent) for at least 2 weeks prior to Cycle 1, Day 1, if applicable; and c) follow-up magnetic resonance imaging (MRI) within 4 weeks prior to Cycle 1, Day 1 shows no new lesions appearing.

In some embodiments, the subject has not undergone a major surgery within 4 weeks prior to the enrollment for the treatment with the compound of formula (I) or (10b) in combination with the EGFR inhibitor, provided that the surgery or procedure is other than peripherally inserted central catheter line placement, thoracentesis, paracentesis, biopsies, or abscess drainage.

In some embodiments, the subject does not have a history of hypersensitivity to the EGFR inhibitor or the compound of formula (I) or (10b), active or inactive excipients of the EGFR inhibitor or the compound of formula (I) or (10b) or drugs with a similar chemical structure or class to either the EGFR inhibitor or the compound of formula (I) or (10b), dependent on which combination the subject could receive.

In some embodiments, the subject is not previously treated with a PTPN11 inhibitor (e.g., SHP2 inhibitor), provided that the PTPN11 inhibitor is other than the compound of formula (I) or (10b). In some embodiments, the subject is not previously treated with a PTPN11 inhibitor selected from the group consisting of TNO-155, RMC-4630, RLY-1971, JAB-3068, JAB-3312, PF-07284892, and ERAS601. In some embodiments, the subject is not previously treated with the compound of formula (I) or (10b). In some embodiments, the subject has previously been treated with a SHP2 inhibitor including any one of TNO-155, RMC-4630, RLY-1971, JAB-3068, JAB-3312, PF-07284892, ERAS601, and the compound of formula (I) or (10b). In some embodiments, the subject has previously been treated with the compound of formula (I) or (10b).

In some embodiments, the subject is not previously treated with an EGFR inhibitor (e.g., osimertinib or erlotinib). In some embodiments, the subject is not previously treated with osimertinib or erlotinib. In some embodiments, the subject is not previously treated with osimertinib. In some embodiments, the subject is not previously treated with erlotinib. In some embodiments, the subject has previously been treated with an EGFR inhibitor (e.g., osimertinib or erlotinib). In some embodiments, the subject has previously been treated with osimertinib or erlotinib. In some embodiments, the subject has previously been treated with osimertinib. In some embodiments, the subject has previously been treated with erlotinib.

In some embodiments, the subject does not have a gastrointestinal illness (e.g., post gastrectomy, short bowel syndrome, uncontrolled Crohn's disease, celiac disease with villous atrophy, or chronic gastritis), which may preclude absorption of the compound of formula (I) or (10b).

In some embodiments, the subject is not on dialysis.

In some embodiments, the subject does not have a history of allogenic bone marrow transplant.

Further inclusion and exclusion criteria for subjects who may benefit from treatment with the compound of formula (I) or (10b) in combination with the EGFR inhibitor, such as subjects enrolled in a clinical study of the SHP2 Inhibitor Compound (10b) in combination with an EGFR inhibitor, are described in Example 4.

In some embodiments, the subject meets all of inclusion criteria of 1) to 7) as described in Example 4. In some embodiments, the subject meets all of inclusion criteria of 1) to 7) as described in Example 4, provided that the subject does not meet any one of exclusion criteria of 1) to 19) as described in Example 4.

### III-4: Treatment Cycle and Dose Adjustment

Treatment with the compound of formula (I) or (10b) in combination with the EGFR inhibitor can include one or more treatment cycles (e.g., at least 1, 2, 3, or more treatment cycles). In some embodiments, the treatment includes one or more treatment cycles (e.g., at least 1, 2, 3, or more treatment cycles). In some embodiments, the treatment includes at least 2, 3, or more treatment cycles. In some embodiments, the treatment includes 2 to 3 treatment cycles. In some embodiments, the treatment includes 3 treatment cycles. In some embodiments, the treatment includes more than 3 treatment cycles.

In some embodiments, each of one or more treatment cycles has a duration of about 28 days; and the compound of formula (I) or (10b) is administered daily. In some embodiments, each of one or more treatment cycles has a duration of about 28 days; and the EGFR inhibitor is administered daily. In some embodiments, each of one or more treatment cycles has a duration of about 28 days; the compound of formula (I) or (10b) is administered daily; and the EGFR inhibitor is administered daily.

The treatment includes a dose escalation period, during which, after a previous treatment cycle, a dose of the compound of formula (I) or (10b) or the EGFR inhibitor can be adjusted (e.g., dose escalation or de-escalation) or retained. Dose adjustment may be based at least in part on a safety evaluation (e.g., a dose-limiting toxicity (DLT) assessment).

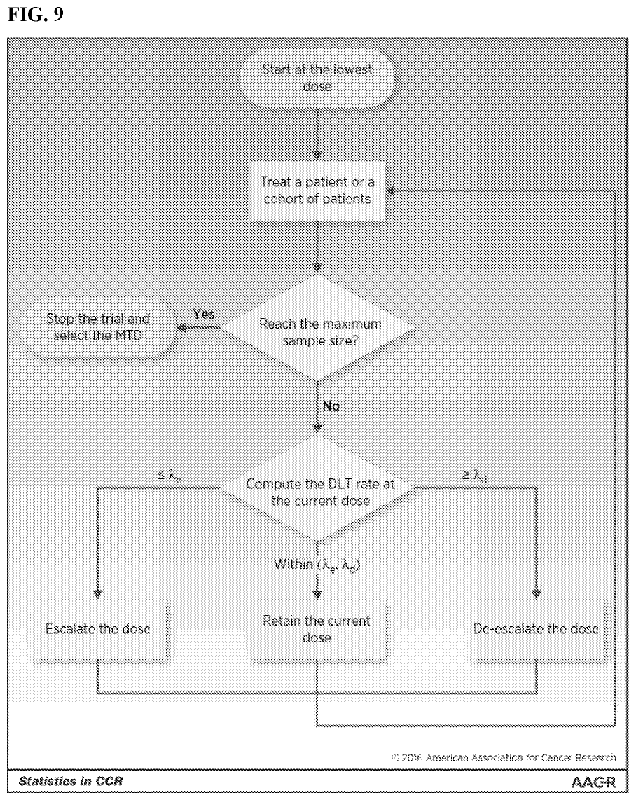

In some embodiments, a subject begins treatment with the compound of formula (I) or (10b) and the EGFR inhibitor at a first compound dose level and a first EGFR inhibitor dose level, and is subsequently treated at a second compound dose level and a second EGFR inhibitor dose level, where the second compound dose level differs from the first compound dose level and/or the second EGFR inhibitor dose level differs from the first EGFR inhibitor dose level. In some embodiments, the second EGFR inhibitor dose level is lower than the first EGFR inhibitor dose level. In some embodiments, the second EGFR inhibitor dose level is higher than the first EGFR inhibitor dose level. In some embodiments, the second compound dose level is lower than the first compound dose level. In some embodiments, the second compound dose level is higher than the first compound dose level. In some embodiments, the administration of EGFR inhibitor in combination with the compound of formula (I) or (10b) includes one or more dose escalations (e.g., dose increases), dose retentions, or dose de-escalations (e.g., dose reduction) of EGFR inhibitor and/or the compound of formula (I) or (10b). In some embodiments, the administration of EGFR inhibitor in combination with the compound of formula (I) or (10b) includes one or more dose escalations, dose retentions, or dose de-escalations of EGFR inhibitor. In some embodiments, the administration of EGFR inhibitor in combination with the compound of formula (I) or (10b) includes one or more dose de-escalations of EGFR inhibitor. In some embodiments, the administration of EGFR inhibitor in combination with the compound of formula (I) or (10b) includes one or more dose escalations of EGFR inhibitor. In some embodiments, the administration of EGFR inhibitor in combination with the compound of formula (I) or (10b) includes one or more dose escalations, dose retentions, or dose de-escalations of the compound of formula (I) or (10b). In some embodiments, the administration of EGFR inhibitor in combination with the compound of formula (I) or (10b) includes one or more dose de-escalations (e.g., dose reductions) of the compound of formula (I) or (10b). In some embodiments, the administration of EGFR inhibitor in combination with the compound of formula (I) or (10b) includes one or more dose escalations (e.g., dose increases) of the compound of formula (I) or (10b). In some embodiments, the administration of EGFR inhibitor in combination with the compound of formula (I) or (10b) includes one or more dose escalations (e.g., dose increases), dose retentions, or dose de-escalations (e.g., dose reductions) of EGFR inhibitor and/or the compound of formula (I) or (10b), each of which is determined by a safety or dose-limiting toxicity (DLT) assessment (e.g., relevant to a cohort of subjects). In some embodiments, the administration of EGFR inhibitor in combination with the compound of formula (I) or (10b) includes one or more dose escalations, dose retentions, or dose de-escalations of the compound of formula (I) or (10b), each of which is determined by a dose-limiting toxicity (DLT) assessment, as described in Example 4 and FIG. 9.

In some embodiments, the administration of the compound of formula (I) or (10b) includes a dose escalation after a previous treatment cycle, when a dose-limiting toxicity (DLT) rate is less than, e.g., about 19.7% as determined by a DLT assessment (e.g., relevant to a cohort of subjects). In some embodiments, the administration of the compound of formula (I) or (10b) includes a dose escalation in a second treatment cycle after a first treatment cycle, when a dose-limiting toxicity (DLT) rate is less than, e.g., about 19.7% as determined by a DLT assessment (e.g., relevant to a cohort of subjects). In some embodiments, the administration of the compound of formula (I) or (10b) includes a dose escalation in a third treatment cycle after a second treatment cycle, when a dose-limiting toxicity (DLT) rate is less than, e.g., about 19.7% as determined by a DLT assessment (e.g., relevant to a cohort of subjects).

In some embodiments, the administration of the compound of formula (I) or (10b) includes a dose de-escalation after a previous treatment cycle, when a dose-limiting toxicity rate is more than, e.g., about 29.8% as determined by a DLT assessment (e.g., relevant to a cohort of subjects). In some embodiments, the administration of the compound of formula (I) or (10b) includes a dose retention in a second treatment cycle after a first treatment cycle, when a dose-limiting toxicity rate is more than, e.g., about 29.8% as determined by a DLT assessment (e.g., relevant to a cohort of subjects). In some embodiments, the administration of the compound of formula (I) or (10b) includes a dose retention in a third treatment cycle after a second treatment cycle, when a dose-limiting toxicity rate is more than, e.g., about 29.8% as determined by a DLT assessment (e.g., relevant to a cohort of subjects).

In some embodiments, the administration of the compound of formula (I) or (10b) includes a dose retention after a previous treatment cycle, when a dose-limiting toxicity rate is in a range of from about 21.9% to about 29.8% as determined by a DLT assessment (e.g., relevant to a cohort of subjects). In some embodiments, the administration of the compound of formula (I) or (10b) includes a dose retention in a second treatment cycle after a first treatment cycle, when a dose-limiting toxicity rate is in a range of from about 21.9% to about 29.8% as determined by a DLT assessment (e.g., relevant to a cohort of subjects). In some embodiments, the administration of the compound of formula (I) or (10b) includes a dose retention in a third treatment cycle after a second treatment cycle, when a dose-limiting toxicity rate is in a range of from about 21.9% to about 29.8% as determined by a DLT assessment (e.g., relevant to a cohort of subjects).

The treatment includes a dose escalation period, during which, after a previous treatment cycle, a dose of the EGFR inhibitor can be adjusted (e.g., dose escalation or de-escalation) or retained. Dose adjustment may be based at least in part on a safety evaluation (e.g., a dose-limiting toxicity (DLT) assessment). In some embodiments, the administration of the EGFR inhibitor includes one or more dose escalations, dose retentions, or dose de-escalations, each of which is determined by a dose-limiting toxicity (DLT) assessment. In some embodiments, the administration of the EGFR inhibitor includes one or more dose escalations, dose retentions, or dose de-escalations, each of which is determined by a dose-limiting toxicity (DLT) assessment, as described herein.

After the dose escalation period, the treatment further includes a dose expansion/optimization period. In some embodiments of the dose expansion/optimization period, the compound of formula (I) or (10b) is administered at at a dose regiment (e.g., Dose Regiment 1 or Dose Regiment 2) determined during the dose escalation period.

In some embodiments, the administration of the compound of formula (I) or (10b) includes one or more dose adjustments. In some embodiments, the administration of the compound of formula (I) or (10b) includes one or more dose adjustments during the dose expansion/optimization period. In some embodiments, the administration of the compound of formula (I) or (10b) includes one or more dose adjustments during the dose expansion/optimization period; and the one or more dose adjustments are determined according to a safety evaluation by Safety Review Committee (SRC).

In any one of embodiments as described herein, the EGFR inhibitor in a total daily dosage is not adjusted (e.g., any dose escalation and/or de-escalation are not allowed during the treatment).

In some embodiments, dosing adjustments, delays, and discontinuations of the compound of formula (I) or (10b) and/or the EGFR inhibitor are further based on the criteria of Example 4.

### III-5: Therapeutically Effective Amount/Administration

The compound of formula (I) or (10b) and the EGFR inhibitor can be provided in jointly therapeutically effective amounts or in synergistically effective amounts, or each of which can be used at a dose different than when each is used alone. In some embodiments, the compound of formula (I) or (10b) and the EGFR inhibitor are provided in jointly therapeutically effective amounts. In some embodiments, the compound of formula (I) or (10b) and the EGFR inhibitor are provided in synergistically effective amounts. In some embodiments, the compound of formula (I) or (10b) and/or the EGFR inhibitor is used at a dose different than when it is used alone (e.g., as in a monotherapy treatment). In some embodiments, the compound of formula (I) or (10b) and the EGFR inhibitor are each used at a dose different than when each is used alone. In some embodiments, the compound of formula (I) or (10b) and the EGFR inhibitor are each used at a dose lower than when each is used alone. In some embodiments, the compound of formula (I) or (10b) is used at a dose lower than when it is used alone. In some embodiments, the EGFR inhibitor is used at a dose lower than when it is used alone. In some embodiments, the compound of formula (I) or (10b) is used at a dose higher than when it is used alone. In some embodiments, the EGFR inhibitor is used at a dose higher than when it is used alone.

The compound of formula (I) or (10b) and the EGFR inhibitor, as defined and described herein, can be administered concomitantly or sequentially. In some embodiments, the compound of formula (I) or (10b) and the EGFR inhibitor are administered concomitantly. In some embodiments, the compound of formula (I) or (10b) and the EGFR inhibitor are administered in a pharmaceutical composition including the compound of formula (I) or (10b) and the EGFR inhibitor. In some embodiments, the compound of formula (I) or (10b) and the EGFR inhibitor are administered sequentially. In some embodiments, the compound of formula (I) or (10b) is administered prior to the administration of the EGFR inhibitor. In some embodiments, the compound of formula (I) or (10b) is administered after the administration of the EGFR inhibitor.

The therapeutically effective amount of the compound of formula (I) or (10b) can be a total daily dosage of no more than about 2000 mg, on a salt-free and anhydrous basis. In some embodiments, the therapeutically effective amount of the compound of formula (I) or (10b) is a total daily dosage of no more than about 2000 mg, on a salt-free and anhydrous basis. In some embodiments, the therapeutically effective amount is a total daily dosage of from about 100 mg to about 2000 mg, from about 150 mg to about 1000 mg, from about 200 mg to about 1000 mg, from about 250 mg to about 1000 mg, from about 300 mg to about 1000 mg, from about 350 mg to about 1000 mg, from about 400 mg to about 1000 mg, from about 450 mg to about 1000 mg, from about 500 mg to about 1000 mg, from about 550 mg to about 1000 mg, from about 600 mg to about 1000 mg, from about 650 mg to about 1000 mg, from about 700 mg to about 1000 mg, from about 100 mg to about 700 mg, from about 150 mg to about 700 mg, from about 200 mg to about 700 mg, from about 250 mg to about 700 mg, from about 300 mg to about 700 mg, from about 350 mg to about 700 mg, from about 400 mg to about 700 mg, from about 450 mg to about 700 mg, from about 500 mg to about 700 mg, from about 550 mg to about 700 mg, from about 100 mg to about 550 mg, from about 150 mg to about 550 mg, from about 200 mg to about 550 mg, from about 250 mg to about 550 mg, from about 300 mg to about 550 mg, from about 350 mg to about 550 mg, from about 400 mg to about 550 mg, from about 450 mg to about 550 mg, from about 100 mg to about 400 mg, from about 150 mg to about 400 mg, from about 200 mg to about 400 mg, from about 250 mg to about 400 mg, or from about 300 mg to about 400 mg of the compound of formula (I) or (10b), on a salt-free and anhydrous basis, or any useful range therein. In some embodiments, the therapeutically effective amount is a total daily dosage of from about 250 mg to about 400 mg, from about 400 mg to about 550 mg, or from about 550 mg to about 700 mg of the compound of formula (I) or (10b), on a salt-free and anhydrous basis, or any useful range therein. In some embodiments, the therapeutically effective amount is a total daily dosage of from about 80 mg to about 700 mg, from about 80 mg to about 550 mg, from about 80 mg to about 400 mg, from about 80 mg to about 250 mg, or from about 80 mg to about 150 mg of the compound of formula (I) or (10b), on a salt-free and anhydrous basis, or any useful range therein.

In some embodiments, the therapeutically effective amount of the compound of formula (I) or (10b) is a total daily dosage of about 100 mg to about 2000 mg, from about 150 mg to about 1000 mg, from about 250 mg to about 1000 mg, from about 400 mg to about 1000 mg, from about 250 mg to about 700 mg, from about 250 mg to about 550 mg, from about 250 mg to about 400 mg, from about 400 mg to about 550 mg, or from about 550 mg to about 700 mg, on a salt-free and anhydrous basis, or any useful range therein. In some embodiments, the therapeutically effective amount is a total daily dosage of from about 250 mg to about 400 mg, from about 400 mg to about 550 mg, or from about 550 mg to about 700 mg of the compound of formula (I) or (10b), on a salt-free and anhydrous basis, or any useful range therein.

The therapeutically effective amount of the compound of formula (10b) can be a total daily dosage of no more than about 2000 mg, on a salt-free and anhydrous basis. In some embodiments, the therapeutically effective amount of the compound of formula (10b) is a total daily dosage of no more than about 2000 mg, on a salt-free and anhydrous basis. In some embodiments, the therapeutically effective amount is a total daily dosage of from about 10 mg to about 2000 mg, from about 50 mg to about 2000 mg, from about 80 mg to about 2000 mg, from about 80 mg to about 1000 mg, from about 80 mg to about 700 mg, from about 80 mg to about 550 mg, from about 80 mg to about 400 mg, from about 80 mg to about 250 mg, or from about 80 mg to about 150 mg of the compound of formula (10b), on a salt-free and anhydrous basis, or any useful range therein. In some embodiments, the therapeutically effective amount is a total daily dosage of from about 80 mg to about 700 mg, from about 80 mg to about 550 mg, from about 80 mg to about 400 mg, from about 80 mg to about 250 mg, or from about 80 mg to about 150 mg of the compound of formula (10b), on a salt-free and anhydrous basis, or any useful range therein.

In some embodiments, the therapeutically effective amount of the compound of formula (10b) is a total daily dosage of about 100 mg to about 2000 mg, from about 150 mg to about 1000 mg, from about 250 mg to about 1000 mg, from about 400 mg to about 1000 mg, from about 250 mg to about 700 mg, from about 250 mg to about 550 mg, from about 250 mg to about 400 mg, from about 400 mg to about 550 mg, or from about 550 mg to about 700 mg of the compound of formula (10b), on a salt-free and anhydrous basis, or any useful range therein. In some embodiments, the therapeutically effective amount is a total daily dosage of from about 250 mg to about 400 mg, from about 400 mg to about 550 mg, or from about 550 mg to about 700 mg, on a salt-free and anhydrous basis, or any useful range therein.

In some embodiments, the therapeutically effective amount of the compound of formula (10b) is a total daily dosage of about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg of the compound of formula (10b), on a salt-free and anhydrous basis. In some embodiments, the therapeutically effective amount is a total daily dosage of about 80 mg, about 150 mg, about 250 mg, about 400 mg, about 550 mg, or about 700 mg of the compound of formula (10b), on a salt-free and anhydrous basis. In some embodiments, the therapeutically effective amount of the compound of formula (10b) is a total daily dosage of about 250 mg, about 400 mg, or about 550 mg of the compound of formula (10b), on a salt-free and anhydrous basis. In some embodiments, the therapeutically effective amount is a total daily dosage of about 80 mg of the compound of formula (10b), on a salt-free and anhydrous basis. In some embodiments, the therapeutically effective amount is a total daily dosage of about 150 mg of the compound of formula (10b), on a salt-free and anhydrous basis. In some embodiments, the therapeutically effective amount is a total daily dosage of about 250 mg of the compound of formula (10b), on a salt-free and anhydrous basis. In some embodiments, the therapeutically effective amount is a total daily dosage of about 400 mg of the compound of formula (10b), on a salt-free and anhydrous basis. In some embodiments, the therapeutically effective amount is a total daily dosage of about 550 mg of the compound of formula (10b), on a salt-free and anhydrous basis. In some embodiments, the therapeutically effective amount is a total daily dosage of about 700 mg of the compound of formula (10b), on a salt-free and anhydrous basis.

The therapeutically effective amount of an EGFR inhibitor (e.g., as described herein) can be a total daily dosage of no more than about 2000 mg of the EGFR inhibitor. In some embodiments, the therapeutically effective amount of the EGFR inhibitor is a total daily dosage of no more than about 2000 mg. In some embodiments, the therapeutically effective amount of the EGFR inhibitor is a total daily dosage of from about 10 mg to about 2000 mg, from about 10 mg to about 1500 mg, from about 10 mg to about 1200 mg, from about 10 mg to about 1000 mg, from about 10 mg to about 960 mg, from about 10 mg to about 840 mg, from about 10 mg to about 800 mg, from about 10 mg to about 720 mg, from about 10 mg to about 600 mg, from about 10 mg to about 480 mg, from about 10 mg to about 360 mg, from about 10 mg to about 300 mg, from about 10 mg to about 240 mg, from about 10 mg to about 150 mg, from about 100 mg to about 2000 mg, from about 100 mg to about 1500 mg, from about 100 mg to about 1200 mg, from about 100 mg to about 1000 mg, from about 100 mg to about 960 mg, from about 100 mg to about 840 mg, from about 100 mg to about 800 mg, from about 100 mg to about 720 mg, from about 100 mg to about 600 mg, from about 100 mg to about 480 mg, from about 100 mg to about 360 mg, from about 100 mg to about 300 mg, from about 100 mg to about 240 mg, from about 100 mg to about 150 mg, from about 200 mg to about 2000 mg, from about 200 mg to about 1500 mg, from about 200 mg to about 1200 mg, from about 200 mg to about 1000 mg, from about 200 mg to about 960 mg, from about 200 mg to about 840 mg, from about 200 mg to about 800 mg, from about 200 mg to about 720 mg, from about 200 mg to about 600 mg, from about 200 mg to about 480 mg, from about 200 mg to about 360 mg, from about 200 mg to about 300 mg, from about 200 mg to about 240 mg, from about 300 mg to about 2000 mg, from about 300 mg to about 1500 mg, from about 300 mg to about 1200 mg, from about 300 mg to about 960 mg, from about 300 mg to about 840 mg, from about 300 mg to about 720 mg, from about 300 mg to about 600 mg, from about 300 mg to about 480 mg, from about 300 mg to about 360 mg, from about 400 mg to about 2000 mg, from about 400 mg to about 1500 mg, from about 400 mg to about 1200 mg, from about 400 mg to about 960 mg, from about 400 mg to about 840 mg, from about 400 mg to about 720 mg, from about 400 mg to about 600 mg, from about 400 mg to about 480 mg, from about 500 mg to about 2000 mg, from about 500 mg to about 1500 mg, from about 500 mg to about 1200 mg, from about 500 mg to about 960 mg, from about 500 mg to about 840 mg, from about 500 mg to about 720 mg, from about 500 mg to about 600 mg, from about 600 mg to about 2000 mg, from about 600 mg to about 1500 mg, from about 600 mg to about 1200 mg, from about 600 mg to about 960 mg, from about 600 mg to about 840 mg, from about 600 mg to about 720 mg, or from about 600 mg to about 1200 mg, or any useful range therein. In some embodiments, the therapeutically effective amount of the EGFR inhibitor is a total daily dosage of about 100 mg, 120 mg, about 150 mg, about 180 mg, about 200 mg, about 240 mg, about 250 mg, about 300 mg, about 350 mg, about 360 mg, about 400 mg, about 450 mg, about 480 mg, about 500 mg, about 550 mg, about 600 mg, about 650 mg, about 700 mg, about 720 mg, about 750 mg, about 800 mg, about 840 mg, about 850 mg, about 900 mg, about 950 mg, about 960 mg, about 1000 mg, about 1050 mg, about 1100 mg, about 1150 mg, or about 1200 mg.

In some embodiments, the therapeutically effective amount of the EGFR inhibitor (e.g., osimertinib) is a total daily dosage of about 80 mg. In some embodiments, the therapeutically effective amount of the EGFR inhibitor (e.g., erlotinib) is a total daily dosage of about 100 mg or about 150 mg. In some embodiments, the therapeutically effective amount of the EGFR inhibitor (e.g., gefitinib) is a total daily dosage of about 250 mg. In some embodiments, the therapeutically effective amount of the EGFR inhibitor (e.g., neratinib) is a total daily dosage of about 240 mg.

In some embodiments, the therapeutically effective amount of the compound of formula (10b) is a total daily dosage of no more than about 2000 mg, on a salt-free and anhydrous basis; and the therapeutically effective amount of the EGFR inhibitor is a total daily dosage of from about 10 mg to about 2000 mg. In some embodiments, the therapeutically effective amount of the compound of formula (10b) is a total daily dosage of about 100 mg to about 2000 mg, from about 150 mg to about 1000 mg, from about 250 mg to about 1000 mg, from about 400 mg to about 1000 mg, from about 250 mg to about 700 mg, from about 250 mg to about 550 mg, from about 250 mg to about 400 mg, from about 400 mg to about 550 mg, from about 550 mg to about 700 mg, or any useful range therein, on a salt-free and anhydrous basis; and the therapeutically effective amount of the EGFR inhibitor is a total daily dosage of from about 10 mg to about 2000 mg. In some embodiments, the therapeutically effective amount of the compound of formula (10b) is a total daily dosage of about 100 mg to about 2000 mg, from about 150 mg to about 1000 mg, from about 250 mg to about 1000 mg, from about 400 mg to about 1000 mg, from about 250 mg to about 700 mg, from about 250 mg to about 550 mg, from about 250 mg to about 400 mg, from about 400 mg to about 550 mg, from about 550 mg to about 700 mg, or any useful range therein, on a salt-free and anhydrous basis; and the therapeutically effective amount of osimertinib is a total daily dosage of about 80 mg. In some embodiments, the therapeutically effective amount of the compound of formula (10b) is a total daily dosage of from about 250 mg to about 400 mg, from about 400 mg to about 550 mg, from about 550 mg to about 700 mg, or any useful range therein, on a salt-free and anhydrous basis; and the therapeutically effective amount of the therapeutically effective amount of osimertinib is a total daily dosage of about 80 mg.

In some embodiments, the therapeutically effective amount of the compound of formula (10b) is a total daily dosage of about 250 mg, about 400 mg, or about 550 mg, on a salt-free and anhydrous basis; and the therapeutically effective amount of the therapeutically effective amount of osimertinib is a total daily dosage of about 80 mg. In some embodiments, the therapeutically effective amount of the compound of formula (10b) is a total daily dosage of about 250 mg, on a salt-free and anhydrous basis; and the therapeutically effective amount of the therapeutically effective amount of osimertinib is a total daily dosage of about 80 mg. In some embodiments, the therapeutically effective amount of the compound of formula (10b) is a total daily dosage of about 400 mg, on a salt-free and anhydrous basis; and the therapeutically effective amount of the therapeutically effective amount of osimertinib is a total daily dosage of about 80 mg. In some embodiments, the therapeutically effective amount of the compound of formula (10b) is a total daily dosage of about 550 mg, on a salt-free and anhydrous basis; and the therapeutically effective amount of the therapeutically effective amount of osimertinib is a total daily dosage of about 80 mg.

In general, the compound of formula (I) or (10b) can be administered orally. In some embodiments, the compound of formula (I) or (10b) is administered orally. In some embodiments, the compound of formula (10b) is administered orally. In some embodiments, the compound of formula (10b) in a tablet formulation is administered orally.

In general, the EGFR inhibitor as defined and described herein can be administered orally. In some embodiments, the EGFR inhibitor is administered orally. In some embodiments, osimertinib is administered orally.

In general, the compound of formula (I) or (10b) can be administered once or multiple times (e.g., 2, 3, 4, or more times) daily. In some embodiments, the compound of formula (I) or (10b) is administered once, twice, three times, or four times daily. In some embodiments, the compound of formula (10b) is administered once, twice, three times, or four times daily. In some embodiments, the compound of formula (10b) is administered once daily. In some embodiments, the compound of formula (10b) is administered twice daily. In some embodiments, the compound of formula (10b) is administered every other day. In some embodiments, the compound of formula (10b) is administered with four days on and three days off (e.g., compound is administered for four consecutive days and then not administered for three consecutive days), five days on and two days off, two days on and five days off, one week on and one week off, two weeks on and one week off, three weeks on and one week off, or a similar schedule.

In general, the EGFR inhibitor can be administered once, twice, or multiple times (e.g., 2, 3, 4, or more times) daily. In some embodiments, the EGFR inhibitor is administered once daily. In some embodiments, osimertinib is administered once daily.

In some embodiments, the compound of formula (I) or (10b) and the EGFR inhibitor are each administered orally. In some embodiments, the compound of formula (10b) and the EGFR inhibitor are each administered orally. In some embodiments, the compound of formula (I) or (10b) is administered once daily; and the EGFR inhibitor is administered once daily. In some embodiments, the compound of formula (10b) is administered once daily; and the EGFR inhibitor is administered once daily. In some embodiments, the compound of formula (10b) is administered once daily; and osimertinib is administered once daily.

The compound of formula (I) or (10b) can be in an oral dosage form in one or more dosage strengths, where the compound of formula (I) or (10b) is present in an amount of at least about 1 mg, 5 mg, 10 mg, 20 mg, 30 mg, 50 mg, 90 mg, 100 mg, 120 mg, 180 mg, 200 mg, 300 mg, 400 mg, or 500 mg, on a salt-free and anhydrous basis. In some embodiments, the oral dosage form is a tablet formulation in one or more dosage strengths. In some embodiments of the tablet formulation, the compound of formula (I) or (10b) is present in an amount of from 1 to 1000 mg, from 1 to 750 mg, from 1 to 500 mg, from 1 to 250 mg, from 30 to 1000 mg, from 30 to 750 mg, from 30 to 500 mg, from 30 to 200 mg, from 30 to 180 mg, from 30 to 120 mg, from 30 to 90 mg, from 50 to 1000 mg, from 50 to 750 mg, from 50 to 500 mg, from 50 to 250 mg, from 100 to 1000 mg, from 100 to 750 mg, from 100 to 500 mg, from 100 to 250 mg, from 200 to 1000 mg, from 200 to 750 mg, from 200 to 500 mg, from 300 to 1000 mg, from 300 to 750 mg, from 300 to 500 mg, from 400 to 1000 mg, from 400 to 750 mg, from 500 to 1000 mg, from 500 to 750 mg, from 600 to 1000 mg, from 5 to 250 mg, or from 5 to 100 mg in each tablet, on a salt-free and anhydrous basis. In some embodiments of the tablet formulation, the compound of formula (I) or (10b) is present in an amount of about 5 mg, 10 mg, 30 mg, 50 mg, 100 mg, 150 mg, 200 mg, 250 mg, 300 mg, 400 mg, 500 mg, 600 mg, 700 mg, 800 mg, 900 mg, or 1000 mg in each tablet, on a salt-free and anhydrous basis. In some embodiments of the tablet formulation, the compound of formula (I) or (10b) is present in an amount of about 30 mg, 50 mg, or 100 mg in each tablet, on a salt-free and anhydrous basis.

The compound of formula (10b) can be in an oral dosage form in one or more dosage strengths, where the compound of formula (10b) is present in an amount of at least about 1 mg, 5 mg, 10 mg, 20 mg, 30 mg, 50 mg, 90 mg, 100 mg, 120 mg, 180 mg, 200 mg, 300 mg, 400 mg, or 500 mg, on a salt-free and anhydrous basis. In some embodiments, the oral dosage form is a tablet formulation in one or more dosage strengths. In some embodiments of the tablet formulation, the compound of formula (10b) is present in an amount of from 1 to 1000 mg, from 1 to 750 mg, from 1 to 500 mg, from 1 to 250 mg, from 30 to 1000 mg, from 30 to 750 mg, from 30 to 500 mg, from 30 to 200 mg, from 30 to 180 mg, from 30 to 120 mg, from 30 to 90 mg, from 50 to 1000 mg, from 50 to 750 mg, from 50 to 500 mg, from 50 to 250 mg, from 100 to 1000 mg, from 100 to 750 mg, from 100 to 500 mg, from 100 to 250 mg, from 200 to 1000 mg, from 200 to 750 mg, from 200 to 500 mg, from 300 to 1000 mg, from 300 to 750 mg, from 300 to 500 mg, from 400 to 1000 mg, from 400 to 750 mg, from 500 to 1000 mg, from 500 to 750 mg, from 600 to 1000 mg, from 5 to 250 mg, or from 5 to 100 mg in each tablet, on a salt-free and anhydrous basis. In some embodiments of the tablet formulation, the compound of formula (10b) is present in an amount of about 5 mg, 10 mg, 30 mg, 50 mg, 100 mg, 150 mg, 200 mg, 250 mg, 300 mg, 400 mg, 500 mg, 600 mg, 700 mg, 800 mg, 900 mg, or 1000 mg in each tablet, on a salt-free and anhydrous basis. In some embodiments of the tablet formulation, the compound of formula (10b) is present in an amount of about 30 mg, 50 mg, or 100 mg in each tablet, on a salt-free and anhydrous basis. In some embodiments of the tablet formulation, the compound of formula (10b) is present in an amount of about 30 mg in each tablet, on a salt-free and anhydrous basis. In some embodiments of the tablet formulation, the compound of formula (10b) is present in an amount of about 50 mg in each tablet, on a salt-free and anhydrous basis. In some embodiments of the tablet formulation, the compound of formula (10b) is present in an amount of about 100 mg in each tablet, on a salt-free and anhydrous basis.

The EGFR inhibitor (e.g., as described herein) can be in an oral dosage form in a dosage strength of, e.g., 40 or 80 mg. In some embodiments, the EGFR inhibitor (e.g., as described herein) is in an oral dosage form in a dosage strength of, e.g., 40 or 80 mg. In some embodiments, osimertinib is in an oral dosage form in a dosage strength of about 80 mg.

In some embodiments, the compound of formula (10b) is administered once daily to provide a total daily dosage of no more than about 2000 mg of the compound of formula (10b). In some embodiments, the compound of formula (10b) is administered once daily to provide a total daily dosage of from about 100 mg to about 2000 mg, from about 150 mg to about 1000 mg, from about 200 mg to about 1000 mg, from about 250 mg to about 1000 mg, from about 300 mg to about 1000 mg, from about 350 mg to about 1000 mg, from about 400 mg to about 1000 mg, from about 450 mg to about 1000 mg, from about 500 mg to about 1000 mg, from about 550 mg to about 1000 mg, from about 600 mg to about 1000 mg, from about 650 mg to about 1000 mg, from about 700 mg to about 1000 mg, from about 100 mg to about 700 mg, from about 150 mg to about 700 mg, from about 200 mg to about 700 mg, from about 250 mg to about 700 mg, from about 300 mg to about 700 mg, from about 350 mg to about 700 mg, from about 400 mg to about 700 mg, from about 450 mg to about 700 mg, from about 500 mg to about 700 mg, from about 550 mg to about 700 mg, from about 100 mg to about 550 mg, from about 150 mg to about 550 mg, from about 200 mg to about 550 mg, from about 250 mg to about 550 mg, from about 300 mg to about 550 mg, from about 350 mg to about 550 mg, from about 250 mg to about 400 mg, from about 400 mg to about 550 mg, from about 450 mg to about 550 mg, from about 100 mg to about 400 mg, from about 150 mg to about 400 mg, from about 200 mg to about 400 mg, from about 250 mg to about 400 mg, from about 300 mg to about 400 mg, or from about 550 mg to about 700 mg of the compound of formula (10b), on a salt-free and anhydrous basis. In some embodiments, the compound of formula (10b) is administered once daily to provide a total daily dosage of from about 250 mg to about 400 mg, from about 400 mg to about 550 mg, or from about 550 mg to about 700 mg of the compound of formula (10b), on a salt-free and anhydrous basis. In some embodiments, the compound of formula (10b) is administered once daily to provide a total daily dosage of about 250 mg, about 400 mg, about 550 mg of the compound of formula (10b), on a salt-free and anhydrous basis.

In some embodiments, the compound of formula (10b) is administered once daily to provide a total daily dosage of no more than about 2000 mg of the compound of formula (10b); and the EGFR inhibitor is administered once daily to provide a total daily dosage of from about 10 mg to about 2000 mg. In some embodiments, the compound of formula (10b) is administered once daily to provide a total daily dosage of from about 100 mg to about 2000 mg, from about 150 mg to about 1000 mg, from about 250 mg to about 1000 mg, from about 400 mg to about 1000 mg, from about 250 mg to about 700 mg, from about 250 mg to about 550 mg, from about 250 mg to about 400 mg, from about 400 mg to about 550 mg, or from about 550 mg to about 700 mg of the compound of formula (10b), on a salt-free and anhydrous basis; and the EGFR inhibitor is administered once daily to provide a total daily dosage of from about 10 mg to about 2000 mg. In some embodiments, the compound of formula (10b) is administered once daily to provide a total daily dosage of from about 250 mg to about 400 mg, from about 400 mg to about 550 mg, or from about 550 mg to about 700 mg of the compound of formula (10b), on a salt-free and anhydrous basis; and osimertinib is administered once daily to provide a total daily dosage of about 80 mg. In some embodiments, the compound of formula (10b) is administered once daily to provide a total daily dosage of about 250 mg, about 400 mg, about 550 mg of the compound of formula (10b), on a salt-free and anhydrous basis; and osimertinib is administered once daily to provide a total daily dosage of about 80 mg.

In some embodiments, the compound of formula (10b) is administered once daily during each of one or more treatment cycles, as described herein. In some embodiments, the EGFR inhibitor is administered once daily during each of one or more treatment cycles, as described herein. In some embodiments, the compound of formula (10b) and the EGFR inhibitor are each administered once daily during each of one or more treatment cycles, as described herein. In some embodiments, the compound of formula (10b) and osimertinib are each administered once daily during each of one or more treatment cycles, as described herein.

In general, the compound of formula (10b) is recommended to be administered to a subject without food (e.g., after an overnight fast (minimum 8 hours) followed by 2 hours of fasting after the dose is taken). The subject is allowed to have water except for one (1) hour before and after the administration and the subject is given with water (e.g., 240 mL) at the administration. In some embodiments, the compound of formula (10b) is administered to the subject without food, at least about 8 hours prior to the administration and at least about 2 hours post the administration.

The EGFR inhibitor may be recommended to be administered to a subject with or without food. In some embodiments, the EGFR inhibitor is administered to the subject with food. In some embodiments, the EGFR inhibitor is administered to the subject without food, at least about 8 hours prior to the administration and at least about 2 hours post the administration.

In some embodiments, the EGFR inhibitor is administered once daily, in about 5 to about 60 minutes after administration of the compound of formula (10b).

### III-6: Efficacy

A clinical study of a PTPN inhibitor (e.g., a compound represented by formula (10b)) in combination with an EGFR inhibitor can be performed to evaluate the safety, tolerability, and efficacy of the combination to reduce or stabilize cancers (e.g., cancers including solid tumors) in subjects. In some embodiments, the subjects have a solid tumor including non-small cell lung cancer (NSCLC). In some embodiments, the subjects have non-small cell lung cancer (NSCLC). In some embodiments, the subjects have NSCLC characterized by an EGFR mutation.

In various embodiments, the subject is administered the therapy for at least 1 month, at least 2 months, at least 3 months, at least 4 months, at least 5 months, at least 6 months, at least 7 months, at least 8 months, at least 9 months, at least 10 months, at least 11 months, at least 12 months, at least 15 months, at least 18 months, at least 21 months, or at least 23 months, e.g., for 1 month, 2 months, 3 months, 4 months, 5 months, 6 months, 7 months, 8 months, 9 months, 10 months, 11 months, 12 months, 15 months, 18 months, 21 months, or 24 months. In various embodiments, the subject is administered the therapy for at least 1 month. In various embodiments, the subject is administered the therapy for at least 3 months. In various embodiments, the subject is administered the therapy for at least 6 months. In various embodiments, the subject is administered the therapy for at least 8 months.

The subject can respond to the therapy as measured by at least a stable disease (SD), as determined by Response Evaluation Criteria in Solid Tumors (RECIST) 1.1 protocol (Eisenhauer, et al., Eur J Cancer; 2009; 45 (2): 228-247). RECIST v1.1 is discussed in detail in the examples below. An at least stable disease is one that is a stable disease, has shown a partial response (PR) or has shown a complete response (CR) (i.e., “at least SD”=SD+PR+CR, often referred to as disease control). In various embodiments, the stable disease has neither sufficient shrinkage to qualify for partial response (PR) nor sufficient increase to qualify for progressive disease (PD). In various embodiments, the subject exhibits at least a partial response (i.e., “at least PR”=PR+CR, often referred to as objective response).

Response can be measured by one or more of decrease in tumor size, suppression or decrease of tumor growth, decrease in target or tumor lesions, delayed time to progression, no new tumor or lesion, a decrease in new tumor formation, an increase in survival or progression-free survival (PFS), and no metastases. In various embodiments, the progression of a subject's disease can be assessed by measuring tumor size, tumor lesions, or formation of new tumors or lesions, by assessing the subject using a computerized tomography (CT) scan, a positron emission tomography (PET) scan, a magnetic resonance imaging (MRI) scan, an X-ray, ultrasound, or some combination thereof.

Progression free survival (PFS) can be assessed as described in the RECIST 1.1 protocol. In various embodiments, the subject exhibits a PFS of at least 1 month. In various embodiments, the subject exhibits a PFS of at least 3 months. In some embodiments, the subject exhibits a PFS of at least 6 months.

Administration of a therapeutically effective amount of the compound of formula (I) or (10b) in combination with a therapeutically effective amount of the EGFR inhibitor can reduce or substantially eliminate cancers or solid tumors in subjects. In some embodiments, the therapeutically effective amount of formula (I) or (10b) in combination with the EGFR inhibitor substantially eliminates the solid tumor. In some embodiments, the therapeutically effective amount of formula (I) or (10b) in combination with the EGFR inhibitor reduces a volume of the solid tumor at least about 10%, about 20%, about 30%, about 40%, about 50%, about 60%, about 70%, about 80%, about 90%, or more. In some embodiments, the therapeutically effective amount of formula (I) or (10b) in combination with the EGFR inhibitor reduces a volume of the solid tumor in a size of from about 10% to about 90%, from about 10% to about 80%, from about 10% to about 70%, from about 10% to about 60%, from about 10% to about 50%, from about 10% to about 40%, from about 10% to about 30%, from about 10% to about 20%, from about 20% to about 90%, from about 20% to about 80%, from about 20% to about 70%, from about 20% to about 60%, from about 20% to about 50%, from about 20% to about 40%, from about 20% to about 30%, from about 30% to about 90%, from about 30% to about 80%, from about 30% to about 70%, from about 30% to about 60%, from about 30% to about 50%, from about 30% to about 40%, from about 40% to about 90%, from about 40% to about 80%, from about 40% to about 70%, from about 40% to about 60%, from about 40% to about 50%, from about 50% to about 90%, from about 50% to about 80%, from about 50% to about 70%, from about 50% to about 60%, from about 60% to about 90%, from about 60% to about 80%, from about 60% to about 70%, from about 70% to about 90%, from about 70% to about 80%, from about 80% to about 90%, or any range therein. In some embodiments, the therapeutically effective amount of formula (I) or (10b) in combination with the EGFR inhibitor reduces a volume of the solid tumor about 10%, about 20%, about 30%, about 40%, about 50%, about 60%, about 70%, about 80%, or about 90%.

Administration of a therapeutically effective amount of the compound of formula (I) or (10b) in combination with a therapeutically effective amount of the EGFR inhibitor can stabilize cancers or solid tumors in subjects. In some embodiments, the therapeutically effective amount of formula (I) or (10b) in combination with the EGFR inhibitor stabilize the solid tumor.

Administration of a therapeutically effective amount of the compound of formula (I) or (10b) in combination with a therapeutically effective amount of the EGFR inhibitor can maintain a reduction or stabilization of cancers or solid tumors in subjects for a period of time (e.g., 1 to 12 months). In some embodiments, the solid tumor is reduced or stabilized for a period of at least about one month with the therapeutically effective amount of the compound of formula (I) or (10b) in combination with the EGFR inhibitor. In some embodiments, the solid tumor is reduced or stabilized for a period of at least about 2, 3, 4, 5, 6, 7, 8, 9, 10, 11, or 12 months with the therapeutically effective amount of the compound of formula (10b) in combination with the EGFR inhibitor. In some embodiments, the solid tumor is reduced or stabilized for a period of from about 1 to about 12 months, from about 1 to about 6 months, from about 1 to about 3 months, or from about 1 to about 2 months.

In some embodiments, the subject is further evaluated to by one or more tests to provide overall assessments including plasma pharmacokinetic and/or pharmacodynamic profiles.

In some embodiments, the subject is further evaluated for one or more biomarkers to determine a correlation of the one or more biomarkers to an antitumor response.

### IV. COMPOUNDS

The present disclosure provides a PTPN11 inhibitor represent by formula (I) for use in a method of treating a disease or disorder (e.g., cancer) in a subject as described in Section III: Combination Therapy, a pharmaceutical composition for treating a disease or disorder (e.g., cancer) in a subject as described in Section V: Composition; and a kit for treating a disease or disorder (e.g., cancer) in a subject as described in Section VI: Kits. The PTPN11 inhibitor is as defined and described in WO 2020/033828, the entirety of which is hereby incorporated for all purpose.

The PTPN11 inhibitor is represent by formula (I):

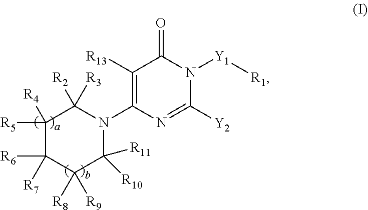

or a pharmaceutically acceptable salt, hydrate, solvate, stereoisomer, conformational isomer, tautomer, or a combination thereof,
wherein:
subscript a is 0 or 1;
subscript b is 0 or 1;
Y₁ is a direct bond or CR₁₇R₁₈;
Y₂ is selected from the group consisting of C₁₋₄alkyl, amino, C₁₋₄alkylC(O)O—, C₁₋₄alkylamino and C₁₋₄aminoalkyl;
R₁ is selected from the group consisting of C₆₋₁₀aryl, C₃₋₈cycloalkyl, C₃₋₈cycloalkenyl, and a 5-10 membered heteroaryl group having 1 to 4 heteroatoms or groups as ring vertices independently selected from N, C(O), O, and S; said aryl or heteroaryl of R₁ is unsubstituted or substituted with 1 to 5 R₁₂ groups independently selected from the group consisting of halo, hydroxy, amino, C₁₋₄alkylamino, di(C₁₋₄alkyl)amino, cyano, C₁₋₄alkyl, C₁₋₄alkoxy, C₁₋₄hydroxyalkyl, C₁₋₄haloalkyl, C₁₋₄aminoalkyl, C₃₋₈cycloalkyl, C₃₋₈cycloalkenyl, NR₁₅C(O) R₁₄, NR₁₅C(O)OR₁₄, NR₁₄C(O)NR₁₅R₁₆, NR₁₅S(O) R₁₄, NR₁₅S(O)₂R₁₄, C(O)NR₁₅R₁₆, S(O)NR₁₅R₁₆, S(O)₂NR₁₅R₁₆, C(O) R₁₄, C(O)OR₁₄, OR₁₄, SR₁₄, S(O) R₁₄, and S(O)₂R₁₄;
R₂, R₃, R₁₀, and R₁₁ are each independently selected from the group consisting of hydrogen, C₁₋₄alkyl, and C₃₋₈cycloalkyl;
R₄, R₅, R₈, and R₉ are each independently selected from the group consisting of hydrogen, cyano, C₁₋₄alkyl, C₁₋₄alkoxy, amino, hydroxy, C₃₋₈cycloalkyl, halo, and C₁₋₄alkylamino;
R₆ is selected from the group consisting of amino, C₁₋₄aminoalkyl, and C₁₋₄alkylamino;
R₇ is selected from the group consisting of hydrogen, amido, cyano, halo, and hydroxy, or is selected from the group consisting of C₁₋₄alkyl, C₁₋₄hydroxyalkyl, C₃₋₆cycloalkyl, phenyl, and 5- or 6-membered heteroaryl, any of which is unsubstituted or substituted with 1 to 5 groups independently selected from the group consisting of amino, halo, hydroxy, cyano, C₁₋₄alkyl, C₁₋₄alkoxy, C₁₋₄haloalkyl, C₁₋₄haloalkoxy, C₁₋₄alkylamino, and C₁₋₄aminoalkyl;
or R₆ and R₇ together with the carbon atom to which they are both attached form a 3- to 7-membered saturated or unsaturated ring, having 0 to 3 heteroatoms or groups as ring vertices independently selected from N, C(O), O, and S(O) m; subscript m is 0, 1, or 2; and said saturated or unsaturated ring formed by R₆ and R₇ is unsubstituted or substituted with 1 to 3 groups independently selected from the group consisting of amino, halo, hydroxy, cyano, C₁₋₄alkyl, C₁₋₄alkoxy, C₁₋₄haloalkyl, C₁₋₄haloalkoxy, C₁₋₄alkylamino and C₁₋₄aminoalkyl;
any two groups of R₂, R₃, R₄, R₅, R₇, R₈, R₉, R₁₀ and R₁₁ can form a 5 to 6 membered ring, having 0 to 2 heteroatoms as ring vertices elected from N, O and S;
any two groups of R₂, R₄, R₆, R₈ and R₁₀ can form a direct bond, or a 1 or 2 atom carbon bridge;
R₁₃ is selected from the group consisting of hydrogen, halo, cyano, C₁₋₆alkyl, C₁₋₆haloalkyl, C₁₋₆hydroxyalkyl, C₁₋₆ dihydroxyalkyl, —NH—NHR₁₉, —NHR₁₉, —OR₁₉, —NHC(O) R₁₉, —NHC(O) NHR₁₉, —NHS(O)₂NHR₁₉, —NHS(O)₂R₁₉, —C(O)OR₁₉, —C(O)NR₁₉R₂₀, —C(O) NH(CH₂)_{q}OH, —C(O) NH(CH₂)_{q}R₂₁, —C(O) R₂₁, —NH₂, —OH, —S(O)₂NR₁₉R₂₀, C₃₋₈cycloalkyl, aryl, heterocyclyl having 1-5 heteroatoms as ring vertices selected from N, O, S and P, and heteroaryl having 1-5 heteroatoms as ring vertices selected from N, O, S and P; subscript q is an integer of from 0 to 6; and each of aryl, heteroaryl, heterocyclyl and cycloalkyl of R₁₃ is unsubstituted or substituted with 1 to 3 groups independently selected from the group consisting of C₁₋₄alkyl, —OH, —NH₂, —OR₂₁, halo, cyano, and oxo;
R₁₄, R₁₅ and R₁₆ are each independently selected from the group consisting of hydrogen, C₁₋₄alkyl, C₃₋₈cycloalkyl, C₆₋₁₀aryl and 5-10 membered heteroaryl, any of which is unsubstituted or substituted with one or more groups independently selected from the group consisting of amido, amino, halo, hydroxy, cyano, C₁₋₄alkyl, C₁₋₄alkoxy, C₁₋₄haloalkyl, C₁₋₄haloalkoxy, C₁₋₄alkylamino and C₁₋₄aminoalkyl;
R₁₇ and R₁₈ are each independently selected from the group consisting of hydrogen, C₁₋₄alkyl, and CF₃;
R₁₉ and R₂₀ are each independently selected from the group consisting of hydrogen, C₁₋₆alkyl, C₁₋₆haloalkyl, C₁₋₆hydroxyalkyl, C₂₋₆alkenyl, C₂₋₆alkynyl and C₃₋₆cycloalkyl; and
each R₂₁ is independently selected from the group consisting of hydrogen, —OH, C₁₋₆ alkyl, C₁₋₆haloalkyl, C₁₋₆hydroxyalkyl, C₂₋₆alkenyl, C₂₋₆alkynyl and C₃₋₆cycloalkyl.

In some embodiments of formula (I), Y₁ is a direct bond. In some embodiments, Y₁ is CR₁₇R₁₈. In some embodiments, R₁₇ and R₁₈ are each independently selected from the group consisting of hydrogen, C₁₋₄alkyl, and CF₃. In some embodiments, R₁₇ and R₁₈ are each independently hydrogen or C₁₋₄alkyl. In some embodiments, Y₁ is-CH₂.

In some embodiments of formula (I), Y₂ is C₁₋₄alkyl. In some embodiments, Y₂ is methyl.

In some embodiments, the compound is represented by formula (Ia):

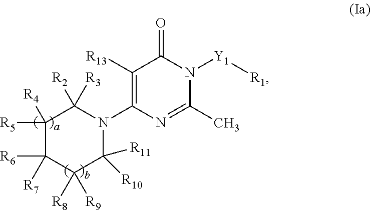

wherein the subscripts a and b, Y₁, R₁, R₂, R₃, R₄, R₅, R₆, R₇, R₈, R₉, R₁₀, R₁₁, and R₁₃ are as defined and described herein.

In some embodiments, the compound is represented by formula (Ib):

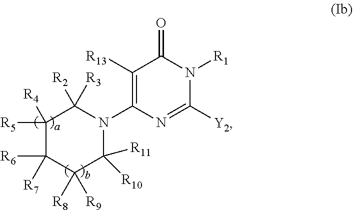

wherein the subscripts a and b, Y₂, R₁, R₂, R₃, R₄, R₅, R₆, R₇, R₈, R₉, R₁₀, R₁₁, and R₁₃ are as defined and described herein.

In some embodiments, the compound is represented by formula (Ic):

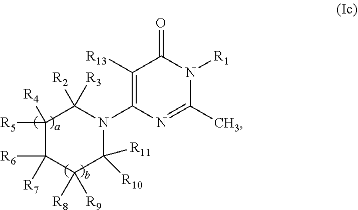

wherein the subscripts a and b, R₁, R₂, R₃, R₄, R₅, R₆, R₇, R₈, R₉, R₁₀, R₁₁, and R₁₃ are as defined and described herein.

In some embodiments of any one of formulae (I), (Ia), (Ib), and (Ic), subscripts a and b are each 1.

In some embodiments of any one of formulae (I), (Ia), (Ib), and (Ic), R₁₃ is selected from the group consisting of hydrogen, halo, C₁₋₆alkyl, C₁₋₆haloalkyl, C₁₋₆hydroxyalkyl, C₁₋₆dihydroxyalkyl, C₃₋₈cycloalkyl, 3- or 6-membered heterocyclyl having 1-3 heteroatoms as ring vertices selected from N, O and S; wherein heterocyclyl and cycloalkyl are substituted with 0 to 3 groups independently selected from the group consisting of C₁₋₄alkyl, —OH, —NH₂, —OR₂₁, halo, cyano and oxo. In some embodiments, R₁₃ is selected from the group consisting of hydrogen, halo, C₁₋₆alkyl, C₁₋₆haloalkyl, C₁₋₆hydroxyalkyl, C₁₋₆dihydroxyalkyl, and C₃₋₈cycloalkyl. In some embodiments, R₁₃ is selected from the group consisting of hydrogen, halo, C₁₋₆alkyl, and C₁₋₆haloalkyl. In some embodiments, R₁₃ is selected from the group consisting of hydrogen, halo, C₁₋₄alkyl, and C₁₋₄haloalkyl. In some embodiments, R₁₃ is selected from the group consisting of —CH₂OH, CF₂OH, and —CHFOH. In some embodiments, R₁₃ is selected from the group consisting of hydrogen, Cl, Br, methyl, and CF₃. In some embodiments, R₁₃ is hydrogen. In some embodiments, R₁₃ is Cl. In some embodiments, R₁₃ is Br. In some embodiments, R₁₃ is methyl. In some embodiments, R₁₃ is CF₃.

In some embodiments of any one of formulae (I), (Ia), (Ib), and (Ic), R₁ is selected from the group consisting of C₆₋₁₀aryl and a 5- to 9-membered heteroaryl group having 1 to 4 heteroatoms groups as ring vertices independently selected from N, C(O), O, and S; and is unsubstituted or substituted with 1 to 5 R₁₂ groups independently selected from the group consisting of halo, hydroxy, amino, C₁₋₄alkylamino, di(C₁₋₄alkyl)amino, cyano, C₁₋₄alkyl, C₁₋₄alkoxy, C₁₋₄hydroxyalkyl, C₁₋₄haloalkyl, C₁₋₄aminoalkyl, C₃₋₈cycloalkyl, C₃₋₈cycloalkenyl, NR₁₅C(O) R₁₄, NR₁₅C(O)OR₁₄, NR₁₄C(O)NR₁₅R₁₆, NR₁₅S(O) R₁₄, NR₁₅S(O)₂R₁₄, C(O)NR₁₅R₁₆, S(O)NR₁₅R₁₆, S(O)₂NR₁₅R₁₆, C(O) R₁₄, C(O)OR₁₄, OR₁₄, SR₁₄, S(O) R₁₄, and S(O)₂R₁₄.

In some embodiments of any one of formulae (I), (Ia), (Ib), and (Ic), R₁ is selected from the group consisting of C₆₋₁₀aryl and a 5- to 9-membered heteroaryl group having 1 to 4 heteroatoms groups as ring vertices independently selected from N, C(O), O, and S; and is unsubstituted or substituted with 1 to 5 R₁₂ groups independently selected from the group consisting of halo, hydroxy, amino, C₁₋₄alkylamino, di(C₁₋₄alkyl)amino, cyano, C₁₋₄alkyl, C₁₋₄alkoxy, C₁₋₄hydroxyalkyl, C₁₋₄haloalkyl, C₁₋₄aminoalkyl, C₃₋₈cycloalkyl, C₃₋₈cycloalkenyl, NR₁₅C(O) R₁₄, NR₁₅C(O)OR₁₄, NR₁₄C(O)NR₁₅R₁₆, NR₁₅S(O) R₁₄, NR₁₅S(O)₂R₁₄, C(O)NR₁₅R₁₆, S(O)NR₁₅R₁₆, S(O)₂NR₁₅R₁₆, C(O) R₁₄, C(O)OR₁₄, OR₁₄, SR₁₄, S(O) R₁₄, and S(O)₂R₁₄; and R₁₄, R₁₅ and R₁₆ are each independently selected from the group consisting of hydrogen, C₁₋₄alkyl, C₃₋₈cycloalkyl, C₆₋₁₀aryl and 5-10 membered heteroaryl, each of which is unsubstituted or substituted by one or more groups independently selected from the group consisting of amido, amino, halo, hydroxy, cyano, C₁₋₄alkyl, C₁₋₄alkoxy, C₁₋₄haloalkyl, C₁₋₄haloalkoxy, C₁₋₄alkylamino and C₁₋₄aminoalkyl.

In some embodiments of any one of formulae (I), (Ia), (Ib), and (Ic), R₁ is selected from the group consisting of C₆₋₁₀aryl and a 5- to 9-membered heteroaryl group having 1 to 4 heteroatoms groups as ring vertices independently selected from N, C(O), O, and S; and is unsubstituted or substituted with 1 to 5 R₁₂ groups independently selected from the group consisting of halo, hydroxy, amino, C₁₋₄alkylamino, di(C₁₋₄alkyl)amino, cyano, C₁₋₄alkyl, C₁₋₄alkoxy, C₁₋₄hydroxyalkyl, C₁₋₄haloalkyl, C₁₋₄aminoalkyl, C₃₋₈cycloalkyl, C₃₋₈cycloalkenyl, NR₁₅C(O) R₁₄, NR₁₅C(O)OR₁₄, NR₁₄C(O)NR₁₅R₁₆, NR₁₅S(O) R₁₄, NR₁₅S(O)₂R₁₄, C(O)NR₁₅R₁₆, S(O)NR₁₅R₁₆, S(O)₂NR₁₅R₁₆, C(O) R₁₄, C(O)OR₁₄, OR₁₄, SR₁₄, S(O) R₁₄, and S(O)₂R₁₄; and R₁₄, R₁₅ and R₁₆ are each independently selected from the group consisting of hydrogen, C₁₋₄alkyl, C₃₋₈cycloalkyl, C₆₋₁₀aryl and 5-10 membered heteroaryl, each of which is unsubstituted or substituted by one or more groups independently selected from the group consisting of halo, hydroxy, cyano, and C₁₋₄alkyl.

In some embodiments of any one of formulae (I), (Ia), (Ib), and (Ic), R₁ is phenyl or a 5- to 6-member heteroaryl group having 1 to 4 heteroatoms as ring vertices independently selected from N, O, and S; and is unsubstituted or substituted with 1, 2 or 3 R₁₂ groups independently selected from the group consisting of halo, hydroxy, amino, C₁₋₄alkylamino, di(C₁₋₄alkyl)amino, C₁₋₄alkyl, C₁₋₄alkoxy, C₁₋₄haloalkyl, C₁₋₄aminoalkyl, and OR₁₄.

In some embodiments of any one of formulae (I), (Ia), (Ib), and (Ic), R₁ is phenyl or a 5- to 6-member heteroaryl group having 1 to 4 heteroatoms as ring vertices independently selected from N, O, and S; and is unsubstituted or substituted with 1, 2 or 3 R₁₂ groups independently selected from the group consisting of halo, hydroxy, amino, C₁₋₄alkylamino, di(C₁₋₄alkyl)amino, C₁₋₄alkyl, C₁₋₄alkoxy, C₁₋₄haloalkyl, C₁₋₄aminoalkyl, and OR₁₄; and R₁₄ is selected from the group consisting of hydrogen, C₁₋₄alkyl, C₃₋₈cycloalkyl, C₆₋₁₀aryl and 5-10 membered heteroaryl, each of which is unsubstituted or substituted by one or more groups independently selected from the group consisting of amido, amino, halo, hydroxy, cyano, C₁₋₄alkyl, C₁₋₄alkoxy, C₁₋₄haloalkyl, C₁₋₄haloalkoxy, C₁₋₄alkylamino and C₁₋₄aminoalkyl.

In some embodiments of any one of formulae (I), (Ia), (Ib), and (Ic), R₁ is phenyl or a 5- to 6-member heteroaryl group having 1 to 4 heteroatoms as ring vertices independently selected from N, O, and S; and is unsubstituted or substituted with 1, 2 or 3 R₁₂ groups independently selected from the group consisting of halo, hydroxy, amino, C₁₋₄alkylamino, di(C₁₋₄alkyl)amino, C₁₋₄alkyl, C₁₋₄alkoxy, C₁₋₄haloalkyl, C₁₋₄aminoalkyl, and OR₁₄; and R₁₄ is selected from the group consisting of hydrogen, C₁₋₄alkyl, C₃₋₈cycloalkyl, C₆₋₁₀aryl and 5-10 membered heteroaryl, each of which is unsubstituted or substituted by one or more groups independently selected from the group consisting of halo, hydroxy, cyano, and C₁₋₄alkyl.

In some embodiments of any one of formulae (I), (Ia), (Ib), and (Ic), R₁ is phenyl or a 5- to 6-member heteroaryl group having 1 to 4 heteroatoms as ring vertices independently selected from N, O, and S; and is unsubstituted or substituted with 1, 2 or 3 R₁₂ groups independently selected from the group consisting of halo, hydroxy, amino, C₁₋₄alkylamino, di(C₁₋₄alkyl)amino, C₁₋₄alkyl, C₁₋₄alkoxy, C₁₋₄haloalkyl, C₁₋₄aminoalkyl, and OR₁₄; and R₁₄ is selected from the group consisting of C₆₋₁₀aryl and a 5-10 membered heteroaryl, each of which is unsubstituted or substituted by one or more groups independently selected from the group consisting of C₁₋₄alkylamido, amino, halo, hydroxy, cyano, C₁₋₄alkyl, C₁₋₄alkoxy, C₁₋₄haloalkyl, C₁₋₄haloalkoxy, C₁₋₄alkylamino and C₁₋₄aminoalkyl.

In some embodiments of any one of formulae (I), (Ia), (Ib), and (Ic), R₁ is phenyl or a 5- to 6-member heteroaryl group having 1 to 4 heteroatoms as ring vertices independently selected from N, O, and S; and is unsubstituted or substituted with 1, 2 or 3 R₁₂ groups independently selected from the group consisting of halo, hydroxy, amino, C₁₋₄alkylamino, di(C₁₋₄alkyl)amino, C₁₋₄alkyl, C₁₋₄alkoxy, C₁₋₄haloalkyl, C₁₋₄aminoalkyl, and OR₁₄; and R₁₄ is selected from the group consisting of C₆₋₁₀aryl and a 5-10 membered heteroaryl, each of which is unsubstituted or substituted by one or more groups independently selected from the group consisting of halo, hydroxy, cyano, and C₁₋₄alkyl.

In some embodiments of any one of formulae (I), (Ia), (Ib), and (Ic), R₁ is phenyl or a 5- to 6-member heteroaryl group having 1 to 4 heteroatoms as ring vertices independently selected from N, O, and S; and is unsubstituted or substituted with 1, 2 or 3 R₁₂ groups independently selected from the group consisting of halo, hydroxy, amino, C₁₋₄alkylamino, di(C₁₋₄alkyl)amino, C₁₋₄alkyl, C₁₋₄alkoxy, C₁₋₄haloalkyl, C₁₋₄aminoalkyl, and OR₁₄; and R₁₄ is phenyl or a 5-6 membered heteroaryl having 1 to 4 heteroatoms as ring vertices independently selected from N, O, and S, each of which is unsubstituted or substituted by one or two groups independently selected from the group consisting of C₁₋₄alkylamido, amino, halo, hydroxy, cyano, C₁₋₄alkyl, C₁₋₄alkoxy, C₁₋₄haloalkyl, C₁₋₄haloalkoxy, C₁₋₄alkylamino and C₁₋₄aminoalkyl.

In some embodiments of any one of formulae (I), (Ia), (Ib), and (Ic), R₁ is phenyl or a 5- to 6-member heteroaryl group having 1 to 4 heteroatoms as ring vertices independently selected from N, O, and S; and is unsubstituted or substituted with 1, 2 or 3 R₁₂ groups independently selected from the group consisting of halo, hydroxy, amino, C₁₋₄alkylamino, di(C₁₋₄alkyl)amino, C₁₋₄alkyl, C₁₋₄alkoxy, C₁₋₄haloalkyl, C₁₋₄aminoalkyl, and OR₁₄; and R₁₄ is phenyl or a 5-6 membered heteroaryl having 1 to 4 heteroatoms as ring vertices independently selected from N, O, and S, each of which is unsubstituted or substituted by one or more groups independently selected from the group consisting of halo, hydroxy, cyano, and C₁₋₄alkyl.

In some embodiments of any one of formulae (I), (Ia), (Ib), and (Ic), R₁ is phenyl or a 5- to 6-member heteroaryl group having 1 to 4 heteroatoms as ring vertices independently selected from N, O, and S; and is unsubstituted or substituted with 1, 2 or 3 R₁₂ groups independently selected from the group consisting of halo, hydroxy, amino, C₁₋₄alkylamino, di(C₁₋₄alkyl)amino, C₁₋₄alkyl, C₁₋₄alkoxy, C₁₋₄haloalkyl, C₁₋₄aminoalkyl, and OR₁₄; and R₁₄ is phenyl or a 5-6 membered heteroaryl having 1 to 4 heteroatoms as ring vertices independently selected from N, O, and S, each of which is unsubstituted or substituted by one or more groups independently selected from the group consisting of C₁₋₄alkylamido, halo, hydroxy, cyano, and C₁₋₄alkyl.

In some embodiments of any one of formulae (I), (Ia), (Ib), and (Ic), R₁ is phenyl or a 5- to 6-member heteroaryl group having 1 to 4 heteroatoms as ring vertices independently selected from N, O, and S; and is unsubstituted or substituted with 1, 2 or 3 R₁₂ groups independently selected from the group consisting of halo, hydroxy, amino, C₁₋₄alkylamino, di(C₁₋₄alkyl)amino, C₁₋₄alkyl, C₁₋₄alkoxy, C₁₋₄haloalkyl, C₁₋₄aminoalkyl, and OR₁₄; and R₁₄ is phenyl or a 5-6 membered heteroaryl having 1 to 4 heteroatoms as ring vertices independently selected from N, O, and S, each of which is unsubstituted or substituted by one or more groups independently selected from the group consisting of C₁₋₄alkylamido and C₁₋₄alkyl.

In some embodiments of any one of formulae (I), (Ia), (Ib), and (Ic), R₁ is phenyl or a 5- to 6-member heteroaryl group having 1 to 4 heteroatoms or groups as ring vertices independently selected from N, C(O), O, and S; and is unsubstituted or substituted with 1, 2 or 3 R₁₂ groups independently selected from the group consisting of halo, hydroxy, amino, C₁₋₄alkylamino, di(C₁₋₄alkyl)amino, C₁₋₄alkyl, C₁₋₄alkoxy, C₁₋₄haloalkyl, and C₁₋₄aminoalkyl.

In some embodiments of any one of formulae (I), (Ia), (Ib), and (Ic), R₂, R₃, R₁₀, and R₁₁ are independently hydrogen or C₁₋₄alkyl. In certain embodiments, R₂, R₃, R₁₀, and R₁₁ are each hydrogen.

In some embodiments of any one of formulae (I), (Ia), (Ib), and (Ic), R₄, R₅, R₈, and R₉ are independently selected from the group consisting of hydrogen, C₁₋₄alkyl, C₁₋₄alkoxy, amino, hydroxy, C₃₋₈cycloalkyl, and C₁₋₄alkylamino. In certain embodiments, R₄, R₅, R₈, and R₉ are independently hydrogen or C₁₋₄alkyl. In certain embodiments, R₄, R₅, R₈, and R₉ are each hydrogen.

In some embodiments of any one of formulae (I), (Ia), (Ib), and (Ic), R₁ is phenyl or a 5- to 6-member heteroaryl group having 1 to 4 heteroatoms or groups as ring vertices independently selected from N, C(O), O, and S; and is unsubstituted or substituted with 1, 2 or 3 R₁₂ groups independently selected from the group consisting of halo, hydroxy, amino, C₁₋₄alkylamino, di(C₁₋₄alkyl)amino, C₁₋₄alkyl, C₁₋₄alkoxy, C₁₋₄haloalkyl, and C₁₋₄aminoalkyl; and R₄, R₅, R₈, and R₉ are independently selected from the group consisting of hydrogen, C₁₋₄alkyl, C₁₋₄alkoxy, amino, hydroxy, C₃₋₆cycloalkyl, and C₁₋₄alkylamino.

In some embodiments of any one of formulae (I), (Ia), (Ib), and (Ic), R₂, R₃, R₄, R₅, R₈, R₉, R₁₀ and R₁₁ are each hydrogen.

In some embodiments, the compound is represented by formula (II):

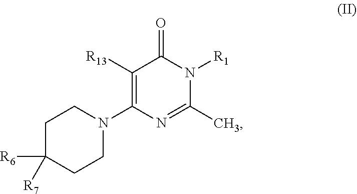

wherein R₁, R₆, R₇, and R₁₃ are as defined and described herein.

In some embodiments of any one of formulae (I), (Ia)-(Ic), and (II), R₆ is selected from the group consisting of amino, C₁₋₄aminoalkyl, and C₁₋₄alkylamino; and R₇ is selected from the group consisting of hydrogen, amido, cyano, halo, and hydroxy, or is selected from the group consisting of C₁₋₄alkyl, C₁₋₄hydroxyalkyl, C₃₋₆cycloalkyl, phenyl, and 5- or 6-membered heteroaryl, any of which is unsubstituted or substituted with one to three substituents selected from the group consisting of amino, halo, hydroxy, cyano, trifluoromethyl, trifluoromethoxy, C₁₋₄alkyl, and C₁₋₄alkoxy. In some embodiments, R₆ is selected from the group consisting of amino, C₁₋₄aminoalkyl, and C₁₋₄alkylamino; and R₇ is selected from the group consisting of hydrogen, amido, halo, and hydroxy, or is selected from the group consisting of C₁₋₄alkyl, C₁₋₄hydroxyalkyl, C₃₋₆cycloalkyl, phenyl, and 5- or 6-membered heteroaryl, any of which is unsubstituted or substituted with one or two substituents selected from the group consisting of amino, halo, hydroxy, cyano, trifluoromethyl, trifluoromethoxy, C₁₋₄alkyl, and C₁₋₄alkoxy.

In some embodiments of any one of formulae (I), (Ia)-(Ic), and (II), R₆ is selected from the group consisting of amino, C₁₋₄aminoalkyl, and methylamino. In some embodiments, R₆ is amino or C₁₋₄aminoalkyl. In certain embodiments, R₆ is amino, aminomethyl, or methylamino. In certain embodiments, R₆ is amino or aminomethyl. In certain embodiments, R₆ is amino. In certain embodiments, R₆ is aminomethyl.

In some embodiments of any one of formulae (I), (Ia)-(Ic), and (II), R₇ is selected from the group consisting of hydroxy, C₁₋₄alkyl, C₁₋₄hydroxyalkyl, C₃₋₆cycloalkyl, phenyl, and 5- or 6-membered heteroaryl, any of which is unsubstituted or substituted with one or two groups selected from the group consisting of amino, halo, hydroxy, cyano, trifluoromethyl, trifluoromethoxy, C₁₋₄alkyl, and C₁₋₄alkoxy. In some embodiments, R₇ is selected from the group consisting of hydroxy, C₁₋₄alkyl, C₁₋₄hydroxyalkyl, C₃₋₆cycloalkyl, phenyl, and 5- or 6-membered heteroaryl. In some embodiments, R₇ is hydroxy, C₁₋₄alkyl, or C₁₋₄hydroxyalkyl. In certain embodiments, R₇ is C₁₋₄alkyl. In certain embodiments, R₇ is methyl. In certain embodiments, R₇ is ethyl.

In some embodiments of any one of formulae (I), (Ia)-(Ic), and (II), R₆ is C₁₋₄aminoalkyl; and R₇ is selected from the group consisting of hydroxy, C₁₋₄alkyl, C₁₋₄hydroxyalkyl, C₃₋₆cycloalkyl, phenyl, and 5- or 6-membered heteroaryl, any of which is unsubstituted or substituted with one to three groups independently selected from the group consisting of amino, halo, hydroxy, cyano, trifluoromethyl, trifluoromethoxy, C₁₋₄alkyl, and C₁₋₄alkoxy.

In some embodiments of any one of formulae (I), (Ia)-(Ic), and (II), R₆ is aminomethyl; and R₇ is selected from the group consisting of hydroxy, C₁₋₄alkyl, C₁₋₄hydroxyalkyl, C₃₋₆cycloalkyl, phenyl, and 5- or 6-membered heteroaryl.

In some embodiments of any one of formulae (I), (Ia)-(Ic), and (II), R₆ is amino; and R₇ is selected from the group consisting of hydroxy, C₁₋₄alkyl, C₁₋₄hydroxyalkyl, C₃₋₆cycloalkyl, phenyl, and 5- or 6-membered heteroaryl, any of which is unsubstituted or substituted with one to three groups independently selected from the group consisting of amino, halo, hydroxy, cyano, trifluoromethyl, trifluoromethoxy, C₁₋₄alkyl, and C₁₋₄alkoxy.

In some embodiments of any one of formulae (I), (Ia)-(Ic), and (II), R₆ is amino; and R₇ is selected from the group consisting of hydroxy, C₁₋₄alkyl, C₁₋₄hydroxyalkyl, C₃₋₆cycloalkyl, phenyl, and 5- or 6-membered heteroaryl.

In some embodiments of any one of formulae (I), (Ia)-(Ic), and (II), R₆ is amino; and R₇ is C₁₋₄hydroxyalkyl. In some embodiments, R₆ is amino; and R₇ is hydroxymethyl. In some embodiments, R₆ is amino; and R₇ is C₁₋₄alkyl. In certain embodiments, R₆ is amino; and R₇ is methyl. In some embodiments, R₆ is amino; and R₇ is ethyl. In some embodiments, R₆ is aminomethyl; and R₇ is C₁₋₄alkyl. In certain embodiments, R₆ is aminomethyl; and R₇ is methyl. In some embodiments, R₆ is aminomethyl; and R₇ is ethyl.

In any of the above embodiments, the amido of R₇ may specifically be —C(O) NH₂.

In some embodiments of any one of formulae (I), (Ia)-(Ic), and (II), R₆ and R₇ together with the carbon atom to which they are both attached form a 3- to 7-membered saturated or unsaturated ring having 1 to 3 heteroatoms or groups as ring vertices independently selected from N, C(O), O, and S(O) m, and that is unsubstituted or substituted with one or two groups independently selected from the group consisting of amino, halo, hydroxy, C₁₋₄alkyl, C₁₋₄alkoxy, C₁₋₄alkylamino and C₁₋₄aminoalkyl.

In some embodiments of any one of formulae (I), (Ia)-(Ic), and (II), R₆ and R₇ together with the carbon atom to which they are both attached form a 4- to 6-membered saturated or unsaturated ring having 1 to 3 heteroatoms or groups as ring vertices independently selected from N, C(O), and O, and that is unsubstituted or substituted with one or two groups independently selected from the group consisting of amino, halo, hydroxy, C₁₋₄alkyl, C₁₋₄alkoxy, C₁₋₄alkylamino and C₁₋₄aminoalkyl.

In some embodiments of any one of formulae (I), (Ia)-(Ic), and (II), R₆ and R₇ together with the carbon atom to which they are both attached form a 3- to 7-membered saturated ring having 1 to 3 heteroatoms or groups as ring vertices independently selected from N, C(O), O, and S(O) m, and that is unsubstituted or substituted with one or two groups independently selected from the group consisting of amino, halo, hydroxy, C₁₋₄alkyl, C₁₋₄alkoxy, C₁₋₄alkylamino and C₁₋₄aminoalkyl.

In some embodiments of any one of formulae (I), (Ia)-(Ic), and (II), R₆ and R₇ together with the carbon atom to which they are both attached form a 4- to 6-membered saturated ring having 1 to 3 heteroatoms or groups as ring vertices independently selected from N, C(O), and O, and that is unsubstituted or substituted with one or two groups independently selected from the group consisting of amino, halo, hydroxy, C₁₋₄alkyl, C₁₋₄alkoxy, C₁₋₄alkylamino and C₁₋₄aminoalkyl.

In some embodiments of any one of formulae (I), (Ia)-(Ic), and (II), R₆ and R₇ together with the carbon atom to which they are both attached form a 4- to 6-membered saturated ring having 1 to 3 heteroatoms or groups as ring vertices independently selected from N and O, and that is unsubstituted or substituted with one or two groups independently selected from the group consisting of amino, halo, hydroxy, C₁₋₄alkyl, C₁₋₄alkoxy, C₁₋₄alkylamino and C₁₋₄aminoalkyl.

In some embodiments of any one of formulae (I), (Ia)-(Ic), and (II), R₆ and R₇ together with the carbon atom to which they are both attached form a 3- to 7-membered cycloalkyl ring that is unsubstituted or substituted with one or two groups independently selected from the group consisting of amino, halo, hydroxy, C₁₋₄alkyl, C₁₋₄alkoxy, C₁₋₄alkylamino and C₁₋₄aminoalkyl.

In some embodiments of any one of formulae (I), (Ia)-(Ic), and (II), R₆ and R₇ together with the carbon atom to which they are both attached form a 4- to 6-membered cycloalkyl ring that is unsubstituted or substituted with one or two groups independently selected from the group consisting of amino, halo, hydroxy, C₁₋₄alkyl, C₁₋₄alkoxy, C₁₋₄alkylamino and C₁₋₄aminoalkyl.

In some embodiments of any one of formulae (I), (Ia)-(Ic), and (II), R₁ is phenyl or a 5- to 6-member heteroaryl group having 1 to 4 heteroatoms as ring vertices independently selected from N, O, and S; and is unsubstituted or substituted with 1, 2 or 3 R₁₂ groups independently selected from the group consisting of halo, hydroxy, amino, C₁₋₄alkylamino, di(C₁₋₄alkyl)amino, C₁₋₄alkyl, C₁₋₄alkoxy, C₁₋₄haloalkyl, C₁₋₄aminoalkyl, and OR₁₄; R₁₄ is phenyl or a 5-6 membered heteroaryl having 1 to 4 heteroatoms as ring vertices independently selected from N, O, and S, each of which is unsubstituted or substituted by one or two groups independently selected from the group consisting of C₁₋₄alkylamido, amino, halo, hydroxy, cyano, C₁₋₄alkyl, C₁₋₄alkoxy, C₁₋₄haloalkyl, C₁₋₄haloalkoxy, C₁₋₄alkylamino and C₁₋₄aminoalkyl; R₆ is selected from the group consisting of amino, C₁₋₄aminoalkyl, and C₁₋₄alkylamino; and R₇ is selected from the group consisting of hydrogen, halo, and hydroxy, or is selected from the group consisting of amido, C₁₋₄alkyl, C₁₋₄hydroxyalkyl, C₃₋₆cycloalkyl, phenyl, and 5- or 6-membered heteroaryl, any of which is unsubstituted or substituted with one or two substituents selected from the group consisting of amino, halo, hydroxy, cyano, trifluoromethyl, trifluoromethoxy, C₁₋₄alkyl, and C₁₋₄alkoxy.

In some embodiments of any one of formulae (I), (Ia)-(Ic), and (II), R₁ is phenyl or a 5- to 6-member heteroaryl group having 1 to 4 heteroatoms as ring vertices independently selected from N, O, and S; and is unsubstituted or substituted with 1, 2 or 3 R₁₂ groups independently selected from the group consisting of halo, hydroxy, amino, C₁₋₄alkylamino, di(C₁₋₄alkyl)amino, C₁₋₄alkyl, C₁₋₄alkoxy, C₁₋₄haloalkyl, C₁₋₄aminoalkyl, and OR₁₄; R₁₄ is phenyl or a 5-6 membered heteroaryl having 1 to 4 heteroatoms as ring vertices independently selected from N, O, and S, each of which is unsubstituted or substituted by one or two groups independently selected from the group consisting of C₁₋₄alkylamido, amino, halo, hydroxy, cyano, C₁₋₄alkyl, C₁₋₄alkoxy, C₁₋₄haloalkyl, C₁₋₄haloalkoxy, C₁₋₄alkylamino and C₁₋₄aminoalkyl; R₆ is amino or aminomethyl; and R₇ is selected from the group consisting of hydroxy, C₁₋₄alkyl, and C₁₋₄hydroxyalkyl.

In some embodiments of any one of formulae (I), (Ia)-(Ic), and (II), R₁ is phenyl or a 5- to 6-member heteroaryl group having 1 to 4 heteroatoms as ring vertices independently selected from N, O, and S; and is unsubstituted or substituted with 1, 2 or 3 R₁₂ groups independently selected from the group consisting of halo, hydroxy, amino, C₁₋₄alkylamino, di(C₁₋₄alkyl)amino, C₁₋₄alkyl, C₁₋₄alkoxy, C₁₋₄haloalkyl, C₁₋₄aminoalkyl, and OR₁₄; R₁₄ is phenyl or a 5-6 membered heteroaryl having 1 to 4 heteroatoms as ring vertices independently selected from N, O, and S, each of which is unsubstituted or substituted by one or two groups independently selected from the group consisting of C₁₋₄alkylamido, amino, halo, hydroxy, cyano, C₁₋₄alkyl, C₁₋₄alkoxy, C₁₋₄haloalkyl, C₁₋₄haloalkoxy, C₁₋₄alkylamino and C₁₋₄aminoalkyl; and R₆ and R₇ together with the carbon atom to which they are both attached form a 3- to 7-membered saturated or unsaturated ring having 1 to 3 heteroatoms or groups as ring vertices independently selected from N, C(O), O, and S(O) m, and that is unsubstituted or substituted with one or two groups independently selected from the group consisting of amino, halo, hydroxy, C₁₋₄alkyl, C₁₋₄alkoxy, C₁₋₄alkylamino and C₁₋₄aminoalkyl.

In some embodiments of any one of formulae (I), (Ia)-(Ic), and (II), R₁ is phenyl or a 5- to 6-member heteroaryl group having 1 to 4 heteroatoms as ring vertices independently selected from N, O, and S; and is unsubstituted or substituted with 1, 2 or 3 R₁₂ groups independently selected from the group consisting of halo, hydroxy, amino, C₁₋₄alkylamino, di(C₁₋₄alkyl)amino, C₁₋₄alkyl, C₁₋₄alkoxy, C₁₋₄haloalkyl, C₁₋₄aminoalkyl, and OR₁₄; R₁₄ is phenyl or a 5-6 membered heteroaryl having 1 to 4 heteroatoms as ring vertices independently selected from N, O, and S, each of which is unsubstituted or substituted by one or two groups independently selected from the group consisting of C₁₋₄alkylamido, amino, halo, hydroxy, cyano, C₁₋₄alkyl, C₁₋₄alkoxy, C₁₋₄haloalkyl, C₁₋₄haloalkoxy, C₁₋₄alkylamino and C₁₋₄aminoalkyl; and R₆ and R₇ together with the carbon atom to which they are both attached form a 4- to 6-membered saturated or unsaturated ring having 1 to 3 heteroatoms or groups as ring vertices independently selected from N, C(O), and O, and that is unsubstituted or substituted with one or two groups independently selected from the group consisting of amino, halo, hydroxy, C₁₋₄alkyl, C₁₋₄alkoxy, C₁₋₄alkylamino and C₁₋₄aminoalkyl.

In some embodiments of any one of formulae (I), (Ia)-(Ic), and (II), R₁ is phenyl or a 5- to 6-member heteroaryl group having 1 to 4 heteroatoms as ring vertices independently selected from N, O, and S; and is unsubstituted or substituted with 1, 2 or 3 R₁₂ groups independently selected from the group consisting of halo, hydroxy, amino, C₁₋₄alkylamino, di(C₁₋₄alkyl)amino, C₁₋₄alkyl, C₁₋₄alkoxy, C₁₋₄haloalkyl, C₁₋₄aminoalkyl, and OR₁₄; R₁₄ is phenyl or a 5-6 membered heteroaryl having 1 to 4 heteroatoms as ring vertices independently selected from N, O, and S, each of which is unsubstituted or substituted by one or two groups independently selected from the group consisting of C₁₋₄alkylamido, amino, halo, hydroxy, cyano, C₁₋₄alkyl, C₁₋₄alkoxy, C₁₋₄haloalkyl, C₁₋₄haloalkoxy, C₁₋₄alkylamino and C₁₋₄aminoalkyl; and R₆ and R₇ together with the carbon atom to which they are both attached form a 3- to 7-membered cycloalkyl ring that is unsubstituted or substituted with one or two groups independently selected from the group consisting of amino, halo, hydroxy, C₁₋₄alkyl, C₁₋₄alkoxy, C₁₋₄alkylamino and C₁₋₄aminoalkyl.

In some embodiments of any one of formulae (I), (Ia)-(Ic), and (II), R₁ is phenyl or a 5- to 6-member heteroaryl group having 1 to 4 heteroatoms as ring vertices independently selected from N, O, and S; and is unsubstituted or substituted with 1, 2 or 3 R₁₂ groups independently selected from the group consisting of halo, hydroxy, amino, C₁₋₄alkylamino, di(C₁₋₄alkyl)amino, C₁₋₄alkyl, C₁₋₄alkoxy, C₁₋₄haloalkyl, and C₁₋₄aminoalkyl; R₆ is selected from the group consisting of amino, C₁₋₄aminoalkyl, and C₁₋₄alkylamino; and R₇ is selected from the group consisting of hydrogen, halo, and hydroxy, or is selected from the group consisting of amido, C₁₋₄alkyl, C₁₋₄hydroxyalkyl, C₃₋₆cycloalkyl, phenyl, and 5- or 6-membered heteroaryl, any of which is unsubstituted or substituted with one or two substituents selected from the group consisting of amino, halo, hydroxy, cyano, trifluoromethyl, trifluoromethoxy, C₁₋₄alkyl, and C₁₋₄alkoxy.

In some embodiments of any one of formulae (I), (Ia)-(Ic), and (II), R₁ is phenyl or a 5- to 6-member heteroaryl group having 1 to 4 heteroatoms as ring vertices independently selected from N, O, and S; and is unsubstituted or substituted with 1, 2 or 3 R₁₂ groups independently selected from the group consisting of halo, hydroxy, amino, C₁₋₄alkylamino, di(C₁₋₄alkyl)amino, C₁₋₄alkyl, C₁₋₄alkoxy, C₁₋₄haloalkyl, and C₁₋₄aminoalkyl; R₆ is amino or aminomethyl; and R₇ is selected from the group consisting of hydroxy, C₁₋₄alkyl, and C₁₋₄hydroxyalkyl.

In some embodiments of any one of formulae (I), (Ia)-(Ic), and (II), R₁ is phenyl or a 5- to 6-member heteroaryl group having 1 to 4 heteroatoms as ring vertices independently selected from N, O, and S; and is unsubstituted or substituted with 1, 2 or 3 R₁₂ groups independently selected from the group consisting of halo, hydroxy, amino, C₁₋₄alkylamino, di(C₁₋₄alkyl)amino, C₁₋₄alkyl, C₁₋₄alkoxy, C₁₋₄haloalkyl, and C₁₋₄aminoalkyl; and R₆ and R₇ together with the carbon atom to which they are both attached form a 3- to 7-membered saturated or unsaturated ring having 1 to 3 heteroatoms or groups as ring vertices independently selected from N, C(O), O, and S(O) m, and that is unsubstituted or substituted with one or two groups independently selected from the group consisting of amino, halo, hydroxy, C₁₋₄alkyl, C₁₋₄alkoxy, C₁₋₄alkylamino and C₁₋₄aminoalkyl.

In some embodiments of any one of formulae (I), (Ia)-(Ic), and (II), R₁ is phenyl or a 5- to 6-member heteroaryl group having 1 to 4 heteroatoms as ring vertices independently selected from N, O, and S; and is unsubstituted or substituted with 1, 2 or 3 R₁₂ groups independently selected from the group consisting of halo, hydroxy, amino, C₁₋₄alkylamino, di(C₁₋₄alkyl)amino, C₁₋₄alkyl, C₁₋₄alkoxy, C₁₋₄haloalkyl, and C₁₋₄aminoalkyl; and R₆ and R₇ together with the carbon atom to which they are both attached form a 4- to 6-membered saturated or unsaturated ring having 1 to 3 heteroatoms or groups as ring vertices independently selected from N, C(O), and O, and that is unsubstituted or substituted with one or two groups independently selected from the group consisting of amino, halo, hydroxy, C₁₋₄alkyl, C₁₋₄alkoxy, C₁₋₄alkylamino and C₁₋₄aminoalkyl.

In some embodiments of any one of formulae (I), (Ia)-(Ic), and (II), R₁ is phenyl or a 5- to 6-member heteroaryl group having 1 to 4 heteroatoms as ring vertices independently selected from N, O, and S; and is unsubstituted or substituted with 1, 2 or 3 R₁₂ groups independently selected from the group consisting of halo, hydroxy, amino, C₁₋₄alkylamino, di(C₁₋₄alkyl)amino, C₁₋₄alkyl, C₁₋₄alkoxy, C₁₋₄haloalkyl, and C₁₋₄aminoalkyl; and R₆ and R₇ together with the carbon atom to which they are both attached form a 3- to 7-membered cycloalkyl ring that is unsubstituted or substituted with one or two groups independently selected from the group consisting of amino, halo, hydroxy, C₁₋₄alkyl, C₁₋₄alkoxy, C₁₋₄alkylamino and C₁₋₄aminoalkyl.

In some embodiments of any one of formulae (I), (Ia)-(Ic), and (II), R₁ is selected from the group consisting of phenyl, pyridyl, pyrimidinyl, pyrazolyl, pyrazinyl, pyridazinyl, and 1,2,4-triazinyl; and is unsubstituted or substituted with 1, 2, or 3 R₁₂, wherein each R₁₂ is as defined and described herein. In some embodiments, R₁ is selected from the group consisting of phenyl, pyridyl, pyrimidinyl, pyrazinyl, pyridazinyl, and 1,2,4-triazinyl; and is unsubstituted or substituted with 1, 2, or 3 R₁₂, wherein each R₁₂ is as defined and described herein. In some embodiments, R₁ is phenyl or pyridyl; and is unsubstituted or substituted with 1, 2, or 3 R₁₂, wherein each R₁₂ is as defined and described herein.

In some embodiments of any one of formulae (I), (Ia)-(Ic), and (II), R₁ is selected from the group consisting of phenyl, pyridyl, pyrimidinyl, pyrazinyl, pyridazinyl, and 1,2,4-triazinyl; and is unsubstituted or substituted with 1, 2, or 3 R₁₂ independently selected from the group consisting of halo, hydroxy, amino, C₁₋₄alkylamino, di(C₁₋₄alkyl)amino, C₁₋₄alkyl, C₁₋₄alkoxy, C₁₋₄haloalkyl, C₁₋₄aminoalkyl, and OR₁₄.

In some embodiments of any one of formulae (I), (Ia)-(Ic), and (II), R₁ is selected from the group consisting of phenyl, pyridyl, pyrimidinyl, pyrazinyl, pyridazinyl, and 1,2,4-triazinyl; and is unsubstituted or substituted with 1, 2, or 3 R₁₂ independently selected from the group consisting of halo, hydroxy, amino, C₁₋₄alkylamino, di(C₁₋₄alkyl)amino, C₁₋₄alkyl, C₁₋₄alkoxy, C₁₋₄haloalkyl, and C₁₋₄aminoalkyl.

In some embodiments of any one of formulae (I), (Ia)-(Ic), and (II), R₁ is phenyl or pyridyl, each of which is unsubstituted or substituted with 1, 2, or 3 R₁₂ independently selected from the group consisting of halo, hydroxy, amino, C₁₋₄alkylamino, di(C₁₋₄alkyl)amino, C₁₋₄alkyl, C₁₋₄alkoxy, C₁₋₄haloalkyl, C₁₋₄aminoalkyl, and OR₁₄.

In some embodiments of any one of formulae (I), (Ia)-(Ic), and (II), R₁ is phenyl or pyridyl, each of which is unsubstituted or substituted with 1, 2, or 3 R₁₂ independently selected from the group consisting of halo, hydroxy, amino, C₁₋₄alkylamino, di(C₁₋₄alkyl)amino, C₁₋₄alkyl, C₁₋₄alkoxy, C₁₋₄haloalkyl, and C₁₋₄aminoalkyl.

In some embodiments of any one of formulae (I), (Ia)-(Ic), and (II), R₁ is phenyl and is unsubstituted or substituted with 1-3 R₁₂, each of which is independently selected from the group consisting of halo, hydroxy, amino, C₁₋₄alkylamino, di(C₁₋₄alkyl)amino, C₁₋₄alkyl, C₁₋₄alkoxy, C₁₋₄haloalkyl, C₁₋₄aminoalkyl, and OR₁₄. In some embodiments, R₁ is phenyl and is unsubstituted or substituted with 1-3 R₁₂, each of which is independently selected from the group consisting of halo, hydroxy, amino, methylamino, dimethylamino, cyano, C₁₋₄alkyl, C₁₋₄haloalkyl, and C₁₋₄alkoxy. In some embodiments, R₁ is phenyl and is unsubstituted or substituted with 1-3 R₁₂, each of which is independently selected from the group consisting of halo, hydroxy, C₁₋₄alkyl, and C₁₋₄alkoxy.

In some embodiments of any one of formulae (I), (Ia)-(Ic), and (II), R₁ is pyridyl and is unsubstituted or substituted with 1-3 R₁₂, each of which is independently selected from the group consisting of halo, hydroxy, amino, C₁₋₄alkylamino, di(C₁₋₄alkyl)amino, C₁₋₄alkyl, C₁₋₄alkoxy, C₁₋₄haloalkyl, C₁₋₄aminoalkyl, and OR₁₄. In some embodiments, R₁ is pyridyl and is unsubstituted or substituted with 1-3 R₁₂, each of which is independently selected from the group consisting of halo, hydroxy, amino, methylamino, dimethylamino, cyano, C₁₋₄alkyl, C₁₋₄haloalkyl, and C₁₋₄alkoxy. In some embodiments, R₁ is pyridyl and is unsubstituted or substituted with 1-3 R₁₂, each of which is independently selected from the group consisting of halo, hydroxy, C₁₋₄alkyl, and C₁₋₄alkoxy.

In some embodiments of any one of formulae (I), (Ia)-(Ic), and (II), R₁ is selected from the group consisting of:

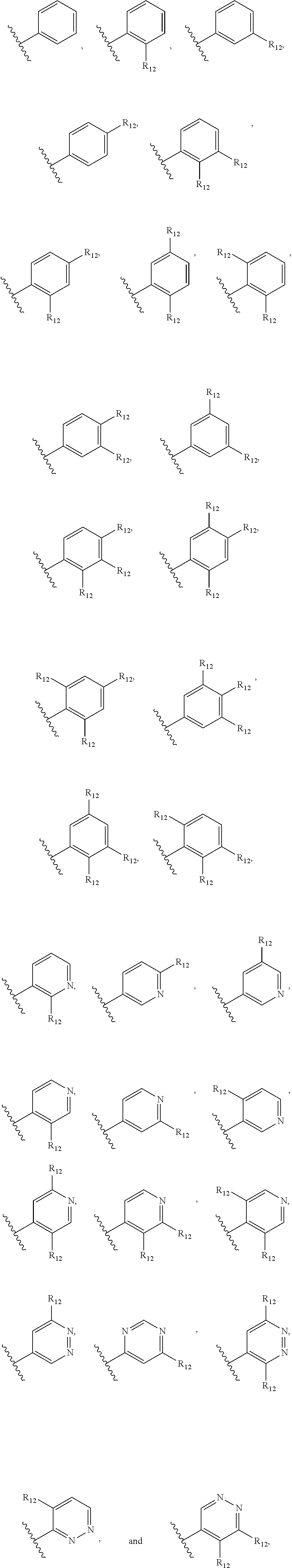

wherein each R₁₂ is as defined and described herein.

In some embodiments of any one of formulae (I), (Ia)-(Ic), and (II), R₁ is selected from the group consisting of:

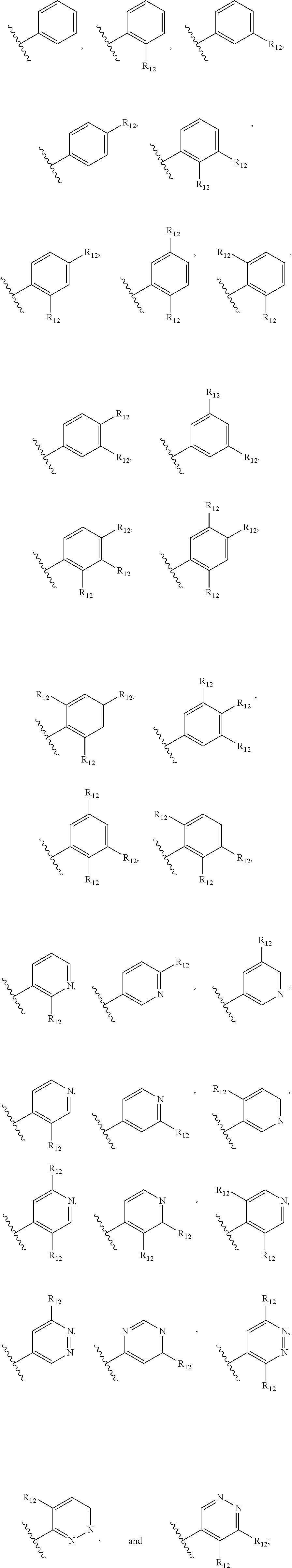

and each R₁₂ is independently selected from the group consisting of halo, hydroxy, amino, C₁₋₄alkylamino, di(C₁₋₄alkyl)amino, C₁₋₄alkyl, C₁₋₄alkoxy, C₁₋₄haloalkyl, C₁₋₄aminoalkyl, and OR₁₄, wherein R₁₄ is as defined and described herein.

In some embodiments of any one of formulae (I), (Ia)-(Ic), and (II), R₁ is selected from the group consisting of:

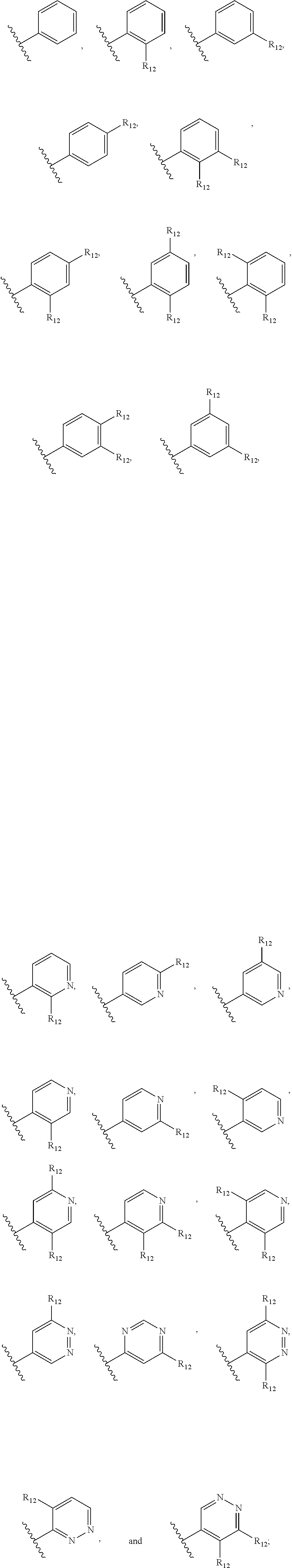

and each R₁₂ is independently selected from the group consisting of halo, hydroxy, amino, C₁₋₄alkylamino, di(C₁₋₄alkyl)amino, C₁₋₄alkyl, C₁₋₄alkoxy, C₁₋₄haloalkyl, and C₁₋₄aminoalkyl.

In some embodiments of any one of formulae (I), (Ia)-(Ic), and (II), R₁ is selected from the group consisting of:

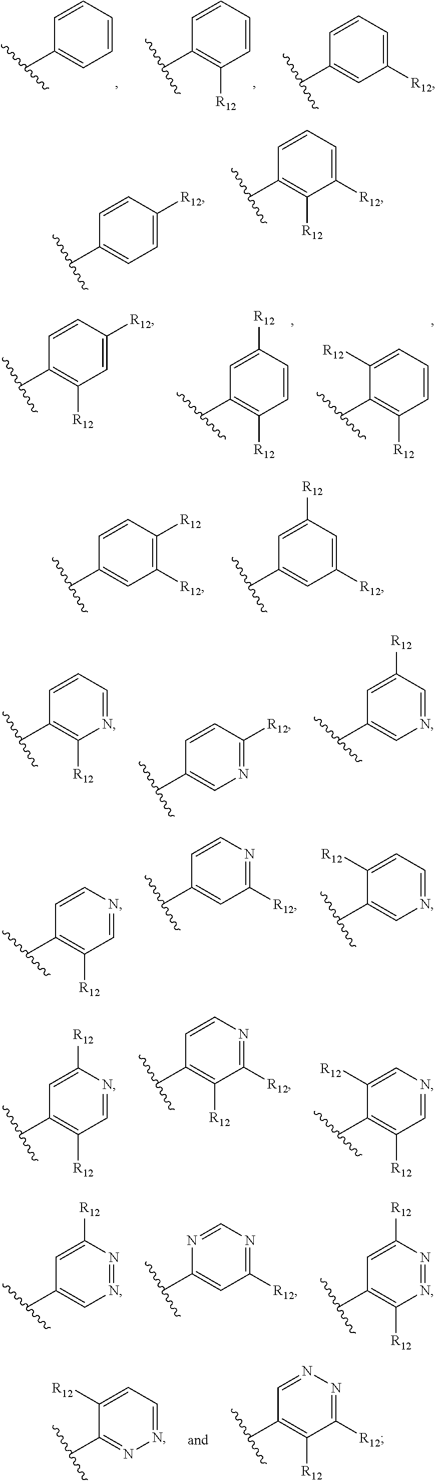

and each R₁₂ is independently selected from the group consisting of halo, hydroxy, amino, methylamino, dimethylamino, cyano, C₁₋₄alkyl, C₁₋₄haloalkyl, and C₁₋₄alkoxy.

In some embodiments of any one of formulae (I), (Ia)-(Ic), and (II), R₁ is selected from the group consisting of:

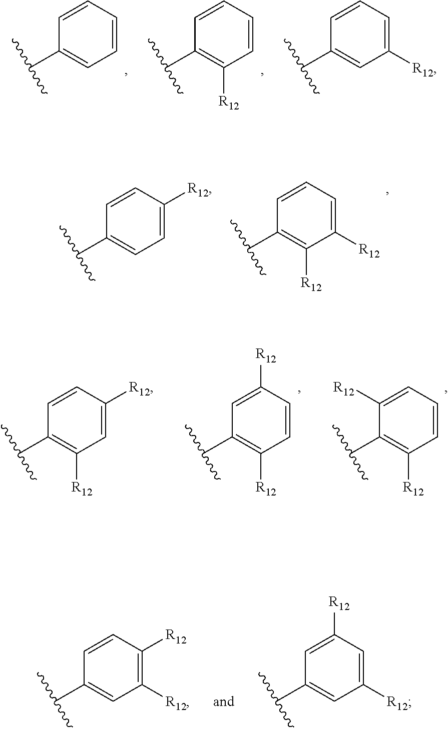

and each R₁₂ is independently selected from the group consisting of halo, hydroxy, amino, methylamino, dimethylamino, cyano, C₁₋₄alkyl, C₁₋₄haloalkyl, and C₁₋₄alkoxy.

In some embodiments of any one of formulae (I), (Ia)-(Ic), and (II), R₁ is selected from the group consisting of:

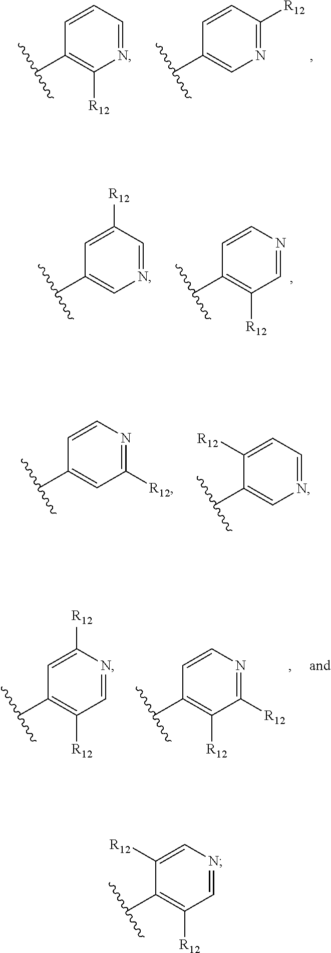

and each R₁₂ is independently selected from the group consisting of halo, hydroxy, amino, methylamino, dimethylamino, cyano, C₁₋₄alkyl, C₁₋₄haloalkyl, and C₁₋₄alkoxy.

In some embodiments of any one of formulae (I), (Ia)-(Ic), and (II), each R₁₂ is independently selected from the group consisting of halo, hydroxy, amino, C₁₋₄alkylamino, di(C₁₋₄alkyl)amino, C₁₋₄alkyl, C₁₋₄alkoxy, C₁₋₄haloalkyl, C₁₋₄aminoalkyl, and OR₁₄. In some embodiments, each R₁₂ is independently selected from the group consisting of halo, C₁₋₄alkyl, C₁₋₄alkoxy, C₁₋₄haloalkyl, and OR₁₄. In some embodiments, each R₁₂ is independently selected from the group consisting of halo, hydroxy, amino, methylamino, dimethylamino, cyano, C₁₋₄alkyl, C₁₋₄haloalkyl, and C₁₋₄alkoxy. In some embodiments, each R₁₂ is independently selected from the group consisting of halo, C₁₋₄alkyl, C₁₋₄alkoxy, and C₁₋₄haloalkyl. In some embodiments, each R₁₂ is independently selected from the group consisting of F, Cl, Br, CH₃, OCH₃, and CF₃.

In some embodiments of any one of formulae (I), (Ia)-(Ic), and (II), each R₁₂ is independently selected from the group consisting of F, Cl, Br, CH₃, OCH₃, CF₃,

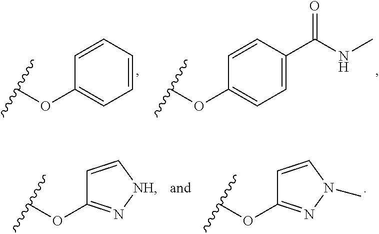

In some embodiments of any one of formulae (I), (Ia)-(Ic), and (II), R₁ is selected from the group consisting of:

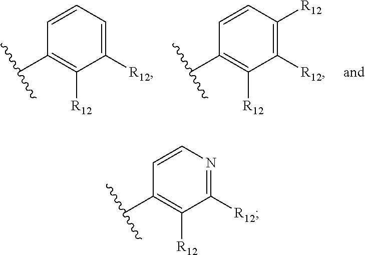

and
each R₁₂ is independently selected from the group consisting of halo, hydroxy, amino, C₁₋₄alkylamino, di(C₁₋₄alkyl)amino, C₁₋₄alkyl, C₁₋₄alkoxy, C₁₋₄haloalkyl, C₁₋₄aminoalkyl, and OR₁₄, wherein R₁₄ is as defined and described herein.

In some embodiments of any one of formulae (I), (Ia)-(Ic), and (II), each R₁₂ is independently selected from the group consisting of F, Cl, Br, CH₃, OCH₃, CF₃, and OR₁₄; and R₁₄ is selected from the group consisting of:

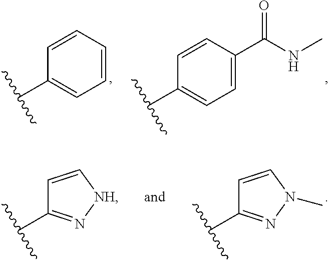

In some embodiments of any one of formulae (I), (Ia)-(Ic), and (II), R₁ is represented by:

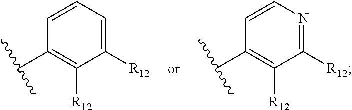

and
each R₁₂ is independently selected from the group consisting of halo, C₁₋₄alkyl, C₁₋₄alkoxy, and C₁₋₄haloalkyl. In some embodiments, each R₁₂ is independently selected from the group consisting of F, Cl, Br, CH₃, OCH₃, and CF₃. In some embodiments, each R₁₂ is Cl.

In some embodiments of any one of formulae (I), (Ia)-(Ic), and (II), R₁ is represented by:

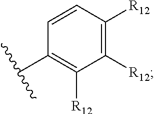

and
each R₁₂ is independently selected from the group consisting of halo, C₁₋₄alkyl, C₁₋₄alkoxy, and C₁₋₄haloalkyl. In some embodiments, each R₁₂ is independently selected from the group consisting of F, Cl, Br, CH₃, OCH₃, and CF₃. In some embodiments, each R₁₂ is independently Cl or Br.

In some embodiments of any one of formulae (I), (Ia)-(Ic), and (II), R₁₄ is independently selected from the group consisting of C₆₋₁₀aryl and a 5-10 membered heteroaryl, each of which is unsubstituted or substituted by one or more groups independently selected from the group consisting of C₁₋₄alkylamido, amino, halo, hydroxy, cyano, C₁₋₄alkyl, C₁₋₄alkoxy, C₁₋₄haloalkyl, C₁₋₄haloalkoxy, C₁₋₄alkylamino and C₁₋₄aminoalkyl. In some embodiments, R₁₄ is independently selected from the group consisting of C₆₋₁₀aryl and a 5-10 membered heteroaryl, each of which is unsubstituted or substituted by one or two groups independently selected from the group consisting of C₁₋₄alkylamido, halo, hydroxy, cyano, and C₁₋₄alkyl. In some embodiments, R₁₄ is independently selected from the group consisting of C₆₋₁₀aryl and a 5-10 membered heteroaryl, each of which is unsubstituted or substituted by one or two groups independently selected from the group consisting of halo, hydroxy, cyano, and C₁₋₄alkyl.

In some embodiments of any one of formulae (I), (Ia)-(Ic), and (II), R₁₄ is independently phenyl or a 5-6 membered heteroaryl having 1 to 4 heteroatoms as ring vertices independently selected from N, O, and S, each of which is unsubstituted or substituted by one or more groups independently selected from the group consisting of C₁₋₄alkylamido, amino, halo, hydroxy, cyano, C₁₋₄alkyl, C₁₋₄alkoxy, C₁₋₄haloalkyl, C₁₋₄haloalkoxy, C₁₋₄alkylamino and C₁₋₄aminoalkyl. In some embodiments, R₁₄ is independently phenyl or a 5-6 membered heteroaryl having 1 to 4 heteroatoms as ring vertices independently selected from N, O, and S, each of which is unsubstituted or substituted by one or two groups independently selected from the group consisting of C₁₋₄alkylamido, halo, hydroxy, cyano, and C₁₋₄alkyl. In some embodiments, R₁₄ is independently phenyl or a 5-6 membered heteroaryl having 1 to 4 heteroatoms as ring vertices independently selected from N, O, and S, each of which is unsubstituted or substituted by one or two groups independently selected from the group consisting of halo, hydroxy, cyano, and C₁₋₄alkyl.

In some embodiments of any one of formulae (I), (Ia)-(Ic), and (II), R₁₄ is phenyl or pyrazolyl, each of which is unsubstituted or substituted by one or two groups independently selected from the group consisting of C₁₋₄alkylamido, halo, hydroxy, cyano, and C₁₋₄alkyl. In some embodiments, R₁₄ is phenyl, unsubstituted or substituted by one or two groups independently selected from the group consisting of C₁₋₄alkylamido, halo, hydroxy, cyano, and C₁₋₄alkyl. In some embodiments, R₁₄ is phenyl, substituted with C₁₋₄alkylamido. In some embodiments, R₁₄ is phenyl substituted with —C(O)NHMe. In some embodiments, R₁₄ is phenyl. In some embodiments, R₁₄ is pyrazolyl, unsubstituted or substituted by one or two groups independently selected from the group consisting of C₁₋₄alkylamido, halo, hydroxy, cyano, and C₁₋₄alkyl. In some embodiments, R₁₄ is pyrazolyl substituted with C₁₋₄alkyl. In some embodiments, R₁₄ is pyrazolyl substituted with methyl. In some embodiments, R₁₄ is N-methylpyrazolyl. In some embodiments, R₁₄ is pyrazolyl.

In some embodiments of any one of formulae (I), (Ia)-(Ic), and (II), R₁ is represented by:

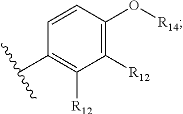

each R₁₂ is independently selected from the group consisting of halo, hydroxy, amino, C₁₋₄alkylamino, di(C₁₋₄alkyl)amino, C₁₋₄alkyl, C₁₋₄alkoxy, C₁₋₄haloalkyl, and C₁₋₄aminoalkyl; and R₁₄ is phenyl or a 5-6 membered heteroaryl having 1 to 4 heteroatoms as ring vertices independently selected from N, O, and S, each of which is unsubstituted or substituted by one or two groups independently selected from the group consisting of C₁₋₄alkylamido, halo, hydroxy, cyano, and C₁₋₄alkyl.

In some embodiments of any one of formulae (I), (Ia)-(Ic), and (II), R₁ is represented by:

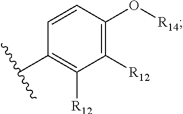

each R₁₂ is independently selected from the group consisting of halo, C₁₋₄alkyl, C₁₋₄alkoxy, C₁₋₄haloalkyl, and OR₁₄; and R₁₄ is phenyl or a 5-6 membered heteroaryl having 1 to 4 heteroatoms as ring vertices independently selected from N, O, and S, each of which is unsubstituted or substituted by one or two groups independently selected from the group consisting of C₁₋₄alkylamido, halo, hydroxy, cyano, and C₁₋₄alkyl.

In some embodiments of any one of formulae (I), (Ia)-(Ic), and (II), R₁ is represented by:

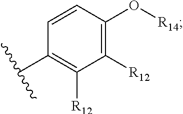

each R₁₂ is independently selected from the group consisting of halo, C₁₋₄alkyl, C₁₋₄alkoxy, C₁₋₄haloalkyl, and OR₁₄; and R₁₄ is selected from the group consisting of phenyl or pyrazolyl, each of which is unsubstituted or substituted by one or two groups independently selected from the group consisting of C₁₋₄alkylamido, halo, hydroxy, cyano, and C₁₋₄alkyl.

In some embodiments of any one of formulae (I), (Ia)-(Ic), and (II), R₁ is represented by:

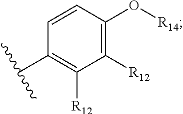

each R₁₂ is independently selected from the group consisting of halo, C₁₋₄alkyl, C₁₋₄alkoxy, C₁₋₄haloalkyl, and OR₁₄; and R₁₄ is selected from the group consisting of phenyl, phenyl substituted with C₁₋₄alkylamido, pyrazolyl, and pyrazolyl substituted with C₁₋₄alkyl. In some embodiments, each R₁₂ is independently selected from the group consisting of F, Cl, Br, CH₃, OCH₃, and CF₃; and R₁₄ is selected from the group consisting of phenyl, MeNHC(O)-phenyl, pyrazolyl, and N-methylpyrazolyl. In some embodiments, each R₁₂ is Cl; and R₁₄ is selected from the group consisting of phenyl, MeNHC(O)-phenyl, pyrazolyl, and N-methylpyrazolyl.

In certain embodiments, the compound is represented by formula (II):

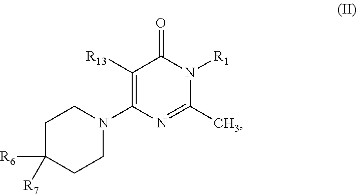

or a salt, ester or prodrug thereof, wherein:
R₁ is phenyl or pyridyl, each of which is substituted with 0 to 3 R₁₂;
each R₁₂ is independently selected from the group consisting of halo, hydroxy, C₁₋₄alkyl, C₁₋₄alkoxy, C₁₋₄haloalkyl, and OR₁₄;
R₆ is selected from the group consisting of amino, C₁₋₄aminoalkyl, and C₁₋₄alkylamino;
R₇ is selected from the group consisting of hydrogen, cyano, amido, halo, and hydroxy, or is selected from the group consisting of C₁₋₄alkyl, C₁₋₄hydroxyalkyl, C₃₋₆cycloalkyl, phenyl, and 5- or 6-membered heteroaryl, any of which is unsubstituted or substituted with one to three substituents independently selected from the group consisting of amino, halo, hydroxy, cyano, trifluoromethyl, trifluoromethoxy, C₁₋₄alkyl, and C₁₋₄alkoxy;
R₁₃ is selected from the group consisting of hydrogen, halo, cyano, C₁₋₆alkyl, C₁₋₆haloalkyl, C₁₋₆hydroxyalkyl, C₁₋₆dihydroxyalkyl, and C₃₋₈cycloalkyl; and
R₁₄ is phenyl or pyrazolyl, each of which is unsubstituted or substituted by one or two groups independently selected from the group consisting of C₁₋₄alkylamido, halo, hydroxy, cyano, and C₁₋₄alkyl.

In certain embodiments, the compound is represented by formula (III):

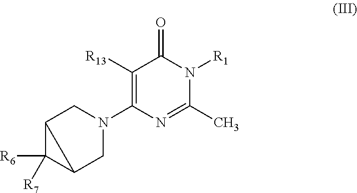

or a salt, ester or prodrug thereof, wherein:
R₁ is phenyl or pyridyl, each of which is substituted with 0 to 3 R₁₂;
each R₁₂ is independently selected from the group consisting of halo, hydroxy, C₁₋₄alkyl, C₁₋₄alkoxy, C₁₋₄haloalkyl, and OR₁₄;
R₆ is selected from the group consisting of amino, C₁₋₄aminoalkyl, and C₁₋₄alkylamino;
R₇ is selected from the group consisting of hydrogen, cyano, amido, halo, and hydroxy, or is selected from the group consisting of C₁₋₄alkyl, C₁₋₄hydroxyalkyl, C₃₋₆cycloalkyl, phenyl, and 5- or 6-membered heteroaryl, any of which is unsubstituted or substituted with one to three substituents independently selected from the group consisting of amino, halo, hydroxy, cyano, trifluoromethyl, trifluoromethoxy, C₁₋₄alkyl, and C₁₋₄alkoxy;
R₁₃ is selected from the group consisting of hydrogen, halo, cyano, C₁₋₆alkyl, C₁₋₆haloalkyl, C₁₋₆hydroxyalkyl, C₁₋₆ dihydroxyalkyl, and C₃₋₈cycloalkyl; and
R₁₄ is phenyl or pyrazolyl, each of which is unsubstituted or substituted by one or two groups independently selected from the group consisting of C₁₋₄alkylamido, halo, hydroxy, cyano, and C₁₋₄alkyl.

In certain embodiments, the compound is represented by formula (IV):

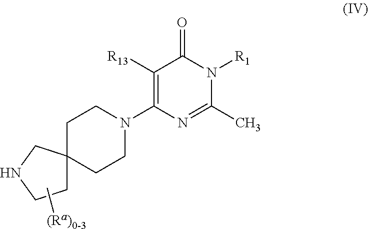

or a salt, ester or prodrug thereof, wherein:
R₁ is phenyl or pyridyl, each of which is substituted with 0 to 3 R₁₂;
each R₁₂ is independently selected from the group consisting of halo, hydroxy, C₁₋₄alkyl, C₁₋₄alkoxy, C₁₋₄haloalkyl, and OR₁₄;
R₁₃ is selected from the group consisting of hydrogen, halo, cyano, C₁₋₆alkyl, C₁₋₆haloalkyl, C₁₋₆hydroxyalkyl, C₁₋₆dihydroxyalkyl, and C₃₋₈cycloalkyl;
R₁₄ is phenyl or pyrazolyl, each of which is unsubstituted or substituted by one or two groups independently selected from the group consisting of C₁₋₄alkylamido, halo, hydroxy, cyano, and C₁₋₄alkyl; and
each R^{a} is independently selected from the group consisting of amino, halo, hydroxy, cyano, C₁₋₄alkyl, C₁₋₄alkoxy, C₁₋₄haloalkyl, C₁₋₄haloalkoxy, C₁₋₄alkylamino and C₁₋₄aminoalkyl.

In certain embodiments, the compound is represented by formula (V):

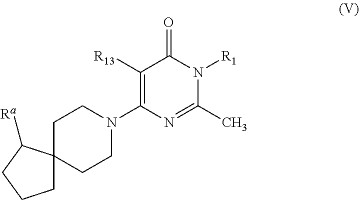

or a salt, ester or prodrug thereof, wherein:
R₁ is phenyl or pyridyl, each of which is substituted with 0 to 3 R₁₂;
each R₁₂ is independently selected from the group consisting of halo, hydroxy, C₁₋₄alkyl, C₁₋₄alkoxy, C₁₋₄haloalkyl, and OR₁₄; and
R₁₃ is selected from the group consisting of hydrogen, halo, cyano, C₁₋₆alkyl, C₁₋₆haloalkyl, C₁₋₆hydroxyalkyl, C₁₋₆dihydroxyalkyl, and C₃₋₈cycloalkyl;
R₁₄ is phenyl or pyrazolyl, each of which is unsubstituted or substituted by one or two groups independently selected from the group consisting of C₁₋₄alkylamido, halo, hydroxy, cyano, and C₁₋₄alkyl; and
R^{a} is selected from the group consisting of amino, halo, hydroxy, cyano, trifluoromethyl, trifluoromethoxy, C₁₋₄alkyl, and C₁₋₄alkoxy.

In certain embodiments, the compound is represented by formula (VI):

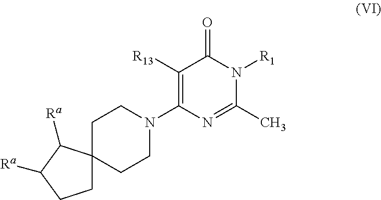

or a salt, ester or prodrug thereof, wherein:
R₁ is phenyl or pyridyl, each of which is substituted with 0 to 3 R₁₂;
each R₁₂ is independently selected from the group consisting of halo, hydroxy, C₁₋₄alkyl, C₁₋₄alkoxy, C₁₋₄haloalkyl, and OR₁₄; and
R₁₃ is selected from the group consisting of hydrogen, halo, cyano, C₁₋₆alkyl, C₁₋₆haloalkyl, C₁₋₆hydroxyalkyl, C₁₋₆dihydroxyalkyl, and C₃₋₈cycloalkyl;
R₁₄ is phenyl or pyrazolyl, each of which is unsubstituted or substituted by one or two groups independently selected from the group consisting of C₁₋₄alkylamido, halo, hydroxy, cyano, and C₁₋₄alkyl; and
each R^{a} is independently selected from the group consisting of amino, halo, hydroxy, cyano, trifluoromethyl, trifluoromethoxy, C₁₋₄alkyl, and C₁₋₄alkoxy.

In certain embodiments, the compound is represented by formula (VII):

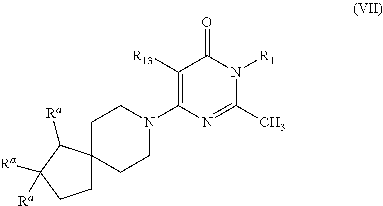

or a salt, ester or prodrug thereof, wherein:
R₁ is phenyl or pyridyl, each of which is substituted with 0 to 3 R₁₂;
each R₁₂ is independently selected from the group consisting of halo, hydroxy, C₁₋₄alkyl, C₁₋₄alkoxy, C₁₋₄haloalkyl, and OR₁₄; and
R₁₃ is selected from the group consisting of hydrogen, halo, cyano, C₁₋₆alkyl, C₁₋₆haloalkyl, C₁₋₆hydroxyalkyl, C₁₋₆dihydroxyalkyl, and C₃₋₈cycloalkyl;
R₁₄ is phenyl or pyrazolyl, each of which is unsubstituted or substituted by one or two groups independently selected from the group consisting of C₁₋₄alkylamido, halo, hydroxy, cyano, and C₁₋₄alkyl; and
each R^{a} is independently selected from the group consisting of amino, halo, hydroxy, cyano, trifluoromethyl, trifluoromethoxy, C₁₋₄alkyl, and C₁₋₄alkoxy.

In certain embodiments, the compound is represented by formula (VIII):

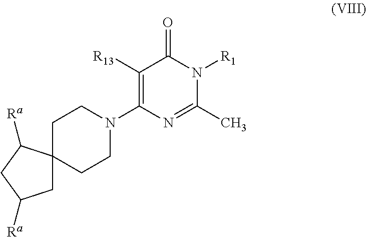

or a salt, ester or prodrug thereof, wherein:
R₁ is phenyl or pyridyl, each of which is substituted with 0 to 3 R₁₂;
each R₁ is independently selected from the group consisting of halo, hydroxy, C₁₋₄alkyl, C₁₋₄alkoxy, C₁₋₄haloalkyl, and OR₁₄; and
R₁₃ is selected from the group consisting of hydrogen, halo, cyano, C₁₋₆alkyl, C₁₋₆haloalkyl, C₁₋₆hydroxyalkyl, C₁₋₆dihydroxyalkyl, and C₃₋₈cycloalkyl;
R₁₄ is phenyl or pyrazolyl, each of which is unsubstituted or substituted by one or two groups independently selected from the group consisting of C₁₋₄alkylamido, halo, hydroxy, cyano, and C₁₋₄alkyl; and
each R^{a} is independently selected from the group consisting of amino, halo, hydroxy, cyano, trifluoromethyl, trifluoromethoxy, C₁₋₄alkyl, and C₁₋₄alkoxy.

In certain embodiments, the compound is represented by formula (IX):

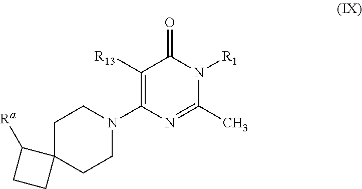

or a salt, ester or prodrug thereof, wherein:
R₁ is phenyl or pyridyl, each of which is substituted with 0 to 3 R₁₂;
each R₁₂ is independently selected from the group consisting of halo, hydroxy, C₁₋₄alkyl, C₁₋₄alkoxy, C₁₋₄haloalkyl, and OR₁₄; and
R₁₃ is selected from the group consisting of hydrogen, halo, cyano, C₁₋₆alkyl, C₁₋₆haloalkyl, C₁₋₆hydroxyalkyl, C₁₋₆dihydroxyalkyl, and C₃₋₈cycloalkyl;
R₁₄ is phenyl or pyrazolyl, each of which is unsubstituted or substituted by one or two groups independently selected from the group consisting of C₁₋₄alkylamido, halo, hydroxy, cyano, and C₁₋₄alkyl; and
R^{a} is selected from the group consisting of amino, halo, hydroxy, cyano, trifluoromethyl, trifluoromethoxy, C₁₋₄alkyl, and C₁₋₄alkoxy.

In certain embodiments, the compound is represented by formula (X):

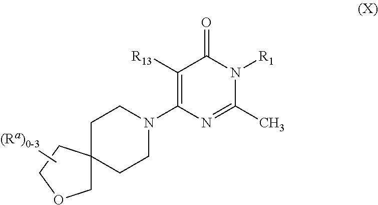

or a salt, ester or prodrug thereof, wherein:
R₁ is phenyl or pyridyl, each of which is substituted with 0 to 3 R₁₂;
each R₁₂ is independently selected from the group consisting of halo, hydroxy, C₁₋₄alkyl, C₁₋₄alkoxy, C₁₋₄haloalkyl, and OR₁₄; and
R₁₃ is selected from the group consisting of hydrogen, halo, cyano, C₁₋₆alkyl, C₁₋₆haloalkyl, C₁₋₆hydroxyalkyl, C₁₋₆dihydroxyalkyl, and C₃₋₈cycloalkyl;
R₁₄ is phenyl or pyrazolyl, each of which is unsubstituted or substituted by one or two groups independently selected from the group consisting of C₁₋₄alkylamido, halo, hydroxy, cyano, and C₁₋₄alkyl; and
each R^{a} is independently selected from the group consisting of amino, halo, hydroxy, cyano, C₁₋₄alkyl, C₁₋₄alkoxy, C₁₋₄haloalkyl, C₁₋₄haloalkoxy, C₁₋₄alkylamino and C₁₋₄aminoalkyl.

In certain embodiments, the compound is represented by formula (XI):

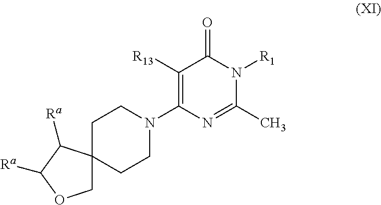

or a salt, ester or prodrug thereof, wherein:
R₁ is phenyl or pyridyl, each of which is substituted with 0 to 3 R₁₂;
each R₁₂ is independently selected from the group consisting of halo, hydroxy, C₁₋₄alkyl, C₁₋₄alkoxy, C₁₋₄haloalkyl, and OR₁₄; and
R₁₃ is selected from the group consisting of hydrogen, halo, cyano, C₁₋₆alkyl, C₁₋₆haloalkyl, C₁₋₆hydroxyalkyl, C₁₋₆dihydroxyalkyl, and C₃₋₈cycloalkyl;
R₁₄ is phenyl or pyrazolyl, each of which is unsubstituted or substituted by one or two groups independently selected from the group consisting of C₁₋₄alkylamido, halo, hydroxy, cyano, and C₁₋₄alkyl; and
each R^{a} is independently selected from the group consisting of amino, halo, hydroxy, cyano, C₁₋₄alkyl, C₁₋₄alkoxy, C₁₋₄haloalkyl, C₁₋₄haloalkoxy, C₁₋₄alkylamino and C₁₋₄aminoalkyl.

In some embodiment of any one of formulae (II)-(XI), R₁, R₆, R₇, R₁₂, R₁₃, and R₁₄ may have the meanings set forth in any one or more of the selected embodiments noted above.

In some embodiments of any one of formulae (II)-(XI), R₁₃ is selected from the group consisting of hydrogen, halo, C₁₋₆alkyl, C₁₋₆haloalkyl, C₁₋₆hydroxyalkyl, C₁₋₆dihydroxyalkyl, and C₃₋₈cycloalkyl. In some embodiments, R₁₃ is selected from the group consisting of hydrogen, halo, C₁₋₆alkyl, and C₁₋₆haloalkyl. In some embodiments, R₁₃ is selected from the group consisting of hydrogen, halo, C₁₋₄alkyl, and C₁₋₄haloalkyl. In some embodiments, R₁₃ is selected from the group consisting of hydrogen, Cl, Br, methyl, and CF₃. In some embodiments, R₁₃ is hydrogen. In some embodiments, R₁₃ is Cl. In some embodiments, R₁₃ is Br. In some embodiments, R₁₃ is methyl. In some embodiments, R₁₃ is CF₃.

In some embodiments of any one of formulae (II)-(XI), R₁ is phenyl or pyridyl, each of which is substituted with 1 to 3 R₁₂. In some embodiments, R₁ is phenyl or pyridyl, each of which is substituted with 2 or 3 R₁₂. In some embodiments, R₁ is phenyl substituted with 2 or 3 R₁₂. In some embodiments, R₁ is phenyl substituted with 2 R₁₂. In some embodiments, R₁ is phenyl substituted with 3 R₁₂. In some embodiments, R₁ is pyridyl substituted with 2 R₁₂.

In some embodiments of any one of formulae (II)-(XI), each R₁₂ is independently selected from the group consisting of halo, C₁₋₄alkyl, C₁₋₄alkoxy, C₁₋₄haloalkyl, and OR₁₄. In some embodiments, each R₁₂ is independently selected from the group consisting of halo, hydroxy, amino, methylamino, dimethylamino, cyano, C₁₋₄alkyl, C₁₋₄haloalkyl, and C₁₋₄alkoxy. In some embodiments, each R₁₂ is independently selected from the group consisting of halo, C₁₋₄alkyl, C₁₋₄alkoxy, and C₁₋₄haloalkyl. In some embodiments, each R₁₂ is independently selected from the group consisting of F, Cl, Br, CH₃, OCH₃, and CF₃.

In some embodiments of any one of formulae (II)-(XI), each R₁₂ is independently selected from the group consisting of F, Cl, Br, CH₃, OCH₃, CF₃,

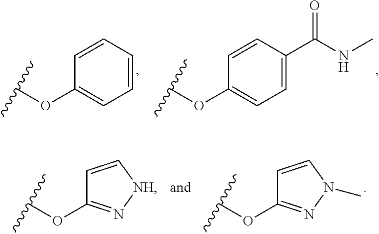

In some embodiments of any one of formulae (II)-(XI), R₁ is phenyl substituted with 2 R₁₂; and each R₁₂ is independently selected from the group consisting of F, Cl, Br, CH₃, OCH₃, and CF₃. In some embodiments, R₁ is phenyl substituted with 2 R₁₂; and each R₁₂ is Cl.

In some embodiments of any one of formulae (II)-(XI), R₁ is phenyl substituted with 3 R₁₂; and each R₁₂ is independently selected from the group consisting of F, Cl, Br, CH₃, OCH₃, CF₃,

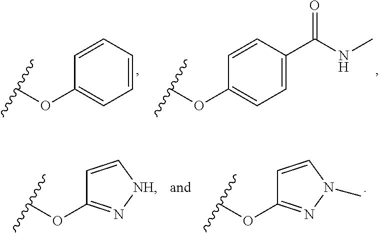

In some embodiments of any one of formulae (II)-(XI), R₁ is phenyl substituted with 3 R₁₂; the first and second R₁₂ are each C₁; and the third R₁₂ is Br. In some embodiments, R₁ is phenyl substituted with 3 R₁₂; the first and second R₁₂ are each Cl; and the third R₁₂ is selected from the group consisting of:

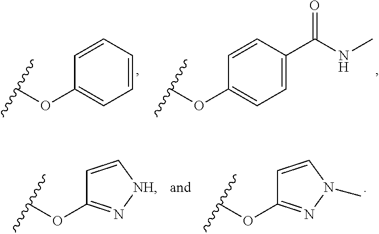

In some embodiments of any one of formulae (II)-(XI), In some embodiments, R₁₄ is phenyl, unsubstituted or substituted by one or two groups independently selected from the group consisting of C₁₋₄alkylamido, halo, hydroxy, cyano, and C₁₋₄alkyl. In some embodiments, R₁₄ is phenyl, substituted with C₁₋₄alkylamido. In some embodiments, R₁₄ is phenyl substituted with —C(O)NHMe. In some embodiments, R₁₄ is phenyl. In some embodiments, R₁₄ is pyrazolyl, unsubstituted or substituted by one or two groups independently selected from the group consisting of C₁₋₄alkylamido, halo, hydroxy, cyano, and C₁₋₄alkyl. In some embodiments, R₁₄ is pyrazolyl substituted with C₁₋₄alkyl. In some embodiments, R₁₄ is pyrazolyl substituted with methyl. In some embodiments, R₁₄ is N-methylpyrazolyl. In some embodiments, R₁₄ is pyrazolyl.

In some embodiments of formula (II) or (III), R₆ is amino or C₁₋₄aminoalkyl. In certain embodiments, R₆ is amino or aminomethyl. In certain embodiments, R₆ is amino. In certain embodiments, R₆ is aminomethyl.

In some embodiments of formula (II) or (III), R₇ is hydroxy, C₁₋₄alkyl, or C₁₋₄hydroxyalkyl. In certain embodiments, R₇ is C₁₋₄alkyl. In certain embodiments, R₇ is methyl. In certain embodiments, R₇ is ethyl.

In some embodiments of formula (II) or (III), R₆ is amino; and R₇ is C₁₋₄alkyl. In certain embodiments, R₆ is amino; and R₇ is methyl. In some embodiments, R₆ is amino; and R₇ is ethyl. In some embodiments, R₆ is aminomethyl; and R₇ is C₁₋₄alkyl. In certain embodiments, R₆ is aminomethyl; and R₇ is methyl.

In some embodiments of any one of formulae (IV)-(XI), each R^{a} is independently selected from the group consisting of amino, halo, hydroxy, cyano, C₁₋₄alkyl, C₁₋₄alkoxy, and C₁₋₄haloalkyl. In some embodiments, each R^{a} is independently amino or C₁₋₄alkyl. In some embodiments, each R^{a} is independently amino or methyl.

In some embodiments, the compound is represented by the formula selected from the group consisting of:

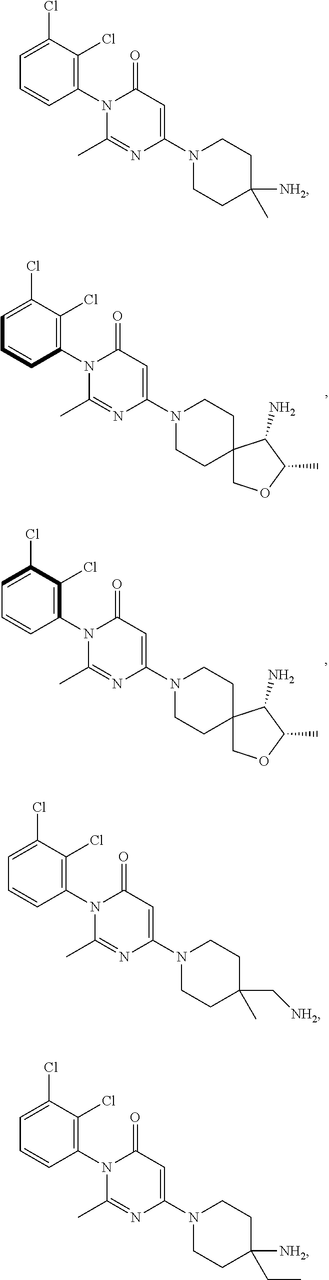

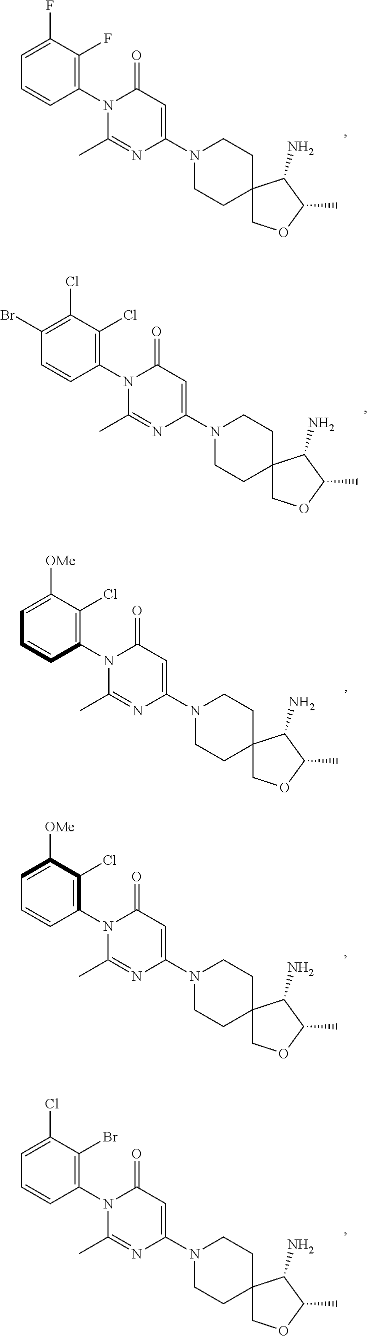

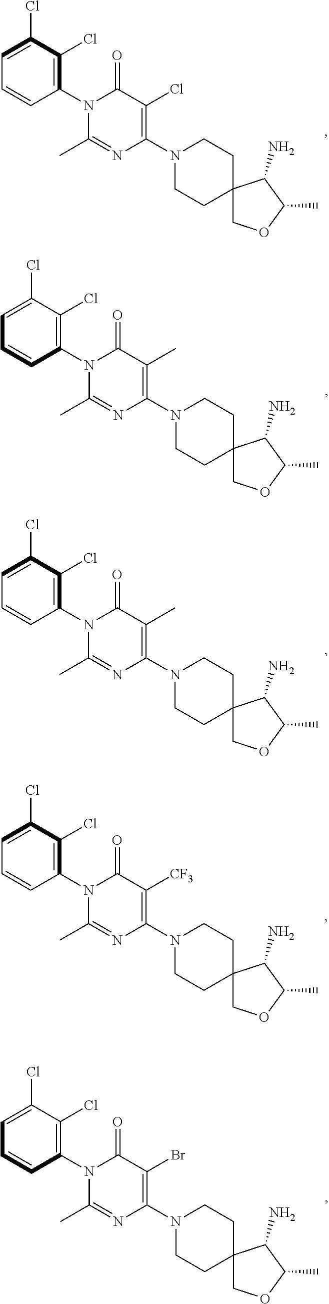

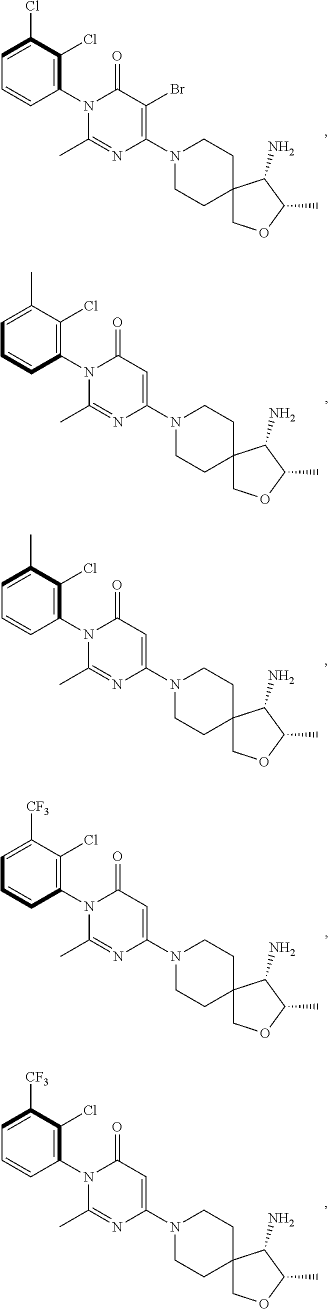

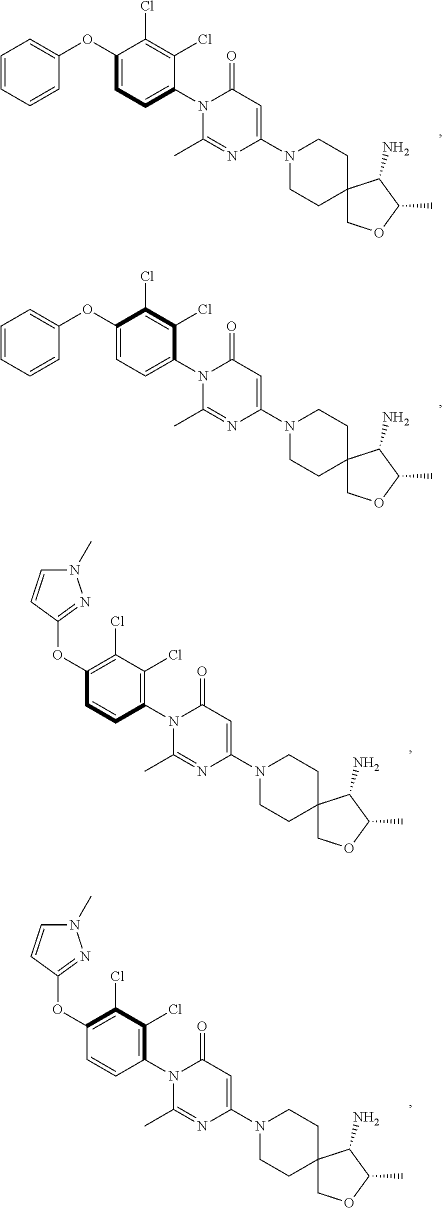

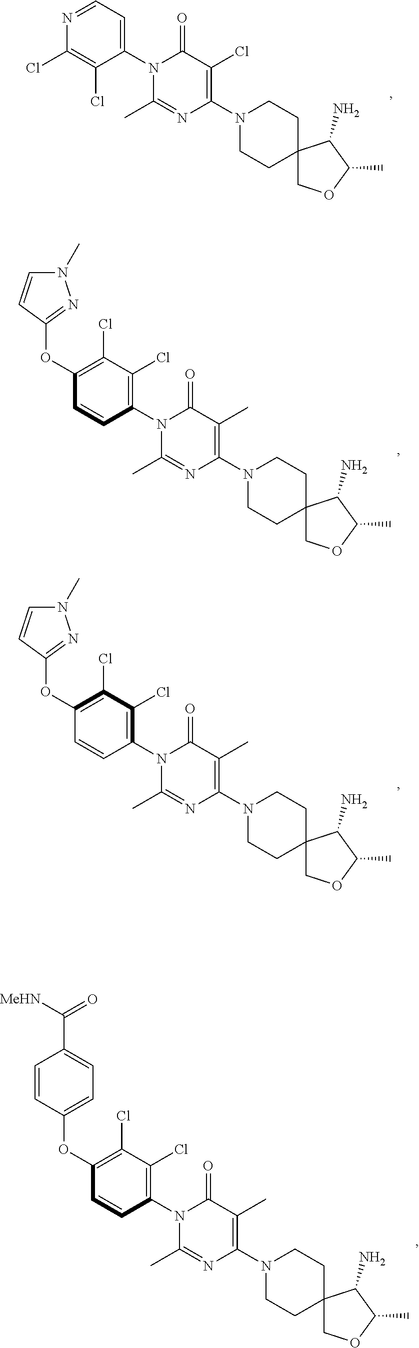

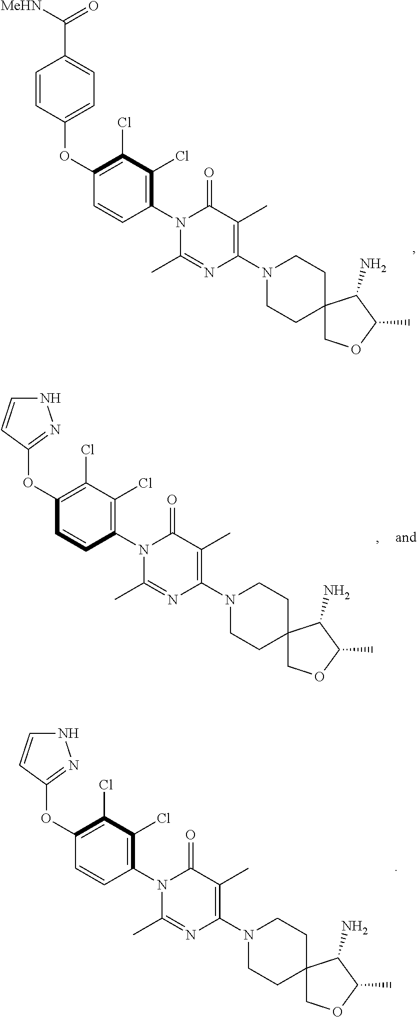

Also provided are embodiments wherein any embodiment above may be combined with any one or more of these embodiments, provided the combination is not mutually exclusive.

As used herein, two embodiments are “mutually exclusive” when one is defined to be something which is different than the other. For example, an embodiment wherein two groups combine to form a cycloalkyl is mutually exclusive with an embodiment in which one group is ethyl the other group is hydrogen. Similarly, an embodiment wherein one group is CH₂ is mutually exclusive with an embodiment wherein the same group is NH.

The compounds disclosed herein can exist as pharmaceutically acceptable salts. The present disclosure includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable.

The term “pharmaceutically acceptable” refers to those compounds (or salts, tautomers, zwitterionic forms, etc.) which are suitable for use in contact with the tissues of patients without undue toxicity, irritation, and allergic response, are commensurate with a reasonable benefit/risk ratio, and are effective for their intended use.

The term “pharmaceutically acceptable salt,” as used herein, represents salts or zwitterionic forms of the compounds disclosed herein which are water or oil-soluble or dispersible and pharmaceutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form pharmaceutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present disclosure contemplates sodium, potassium, magnesium, and calcium salts of the compounds disclosed herein, and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of pharmaceutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N-dibenzylethylenediamine. Other representative organic amines useful for the formation of base addition salts include ethylenediamine, ethanolamine, diethanolamine, piperidine, and piperazine.

A salt of a compound can be made by reacting the appropriate compound in the form of the free base with the appropriate acid.

### V. Composition

The oral dosage form including the compound of formula (I) or (10b) can be in any oral dosage forms including one or more pharmaceutically acceptable carriers and/or excipients. Oral preparations include tablets, pills, powder, dragees, capsules, liquids, lozenges, cachets, gels, syrups, slurries, suspensions, etc., suitable for ingestion by the patient.

For preparing oral dosage forms including the compound of formula (I) or (10b), pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton PA (“Remington's”).

In powders, the carrier is a finely divided solid, which is in a mixture with the finely divided active component. In tablets, the active component is mixed with the carrier having the necessary binding properties in suitable proportions and compacted in the shape and size desired.

The powders, capsules and tablets preferably contain from 5% to 70% of the compound of formula (I) or (10b), or from about 10% to about 70% of the compound of formula (I) or (10b). Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other excipients, is surrounded by a carrier, which is thus in association with it. Similarly, cachets and lozenges are included. Tablets, powders, capsules, pills, cachets, and lozenges can be used as solid dosage forms suitable for oral administration.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins including, but not limited to, gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the dosage forms can also be used orally using, for example, push-fit capsules made of gelatin, as well as soft, sealed capsules made of gelatin and a coating such as glycerol or sorbitol. Push-fit capsules can contain the compound of formula (I) or (10b) mixed with a filler or binders such as lactose or starches, lubricants such as talc or magnesium stearate, and, optionally, stabilizers. In soft capsules, the compound of formula (I) or (10b) may be dissolved or suspended in suitable liquids, such as fatty oils, liquid paraffin, or liquid polyethylene glycol with or without stabilizers.

For preparing suppositories, a low melting wax, such as a mixture of fatty acid glycerides or cocoa butter, is first melted and the compound of formula (I) or (10b) are dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions.

Aqueous solutions suitable for oral use can be prepared by dissolving the compound of formula (I) or (10b) in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending the compound of formula (I) or (10b) in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations including the compound of formula (I) or (10b) can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

In another aspect, the present disclosure provides a pharmaceutical composition for treating a disease or disorder (e.g., cancer) in a subject, the composition including:
a) a therapeutically effective amount of a PTPN11 inhibitor; and
b) a therapeutically effective amount of an EGFR inhibitor,
   together with a pharmaceutically acceptable carrier or excipient,
      wherein the PTPN11 inhibitor is represented by formula (I) as defined and described herein.

The cancer and/or solid tumor are described according to Section III-2: Cancer/Solid Tumor. In some embodiments, the cancer and/or solid tumor are any of embodiments as described in Section III-2: Cancer/Solid Tumor.

The subject is described according to Section III-3: Subject. In some embodiments, the subject is any of embodiments as described in Section III-3: Subject.

The PTPN11 inhibitor represented by formula (I) is described according to Section III-1: PTPN11 Inhibitors and/or EGFR Inhibitors. In some embodiments, the PTPN11 inhibitor of formula (I) is any of embodiments as described in Section III-1: PTPN11 Inhibitors and/or EGFR Inhibitors. In some embodiments, the PTPN11 inhibitor of formula (I) is the compound of formula (10b).

The PTPN11 inhibitor of formula (I) is further described according to Section IV. Compounds. In some embodiments, the PTPN11 inhibitor of formula (I) is any of embodiments as described in Section IV. Compounds.

The EGFR inhibitor is described according to Section III-1: PTPN11 Inhibitors and/or EGFR Inhibitors. In some embodiments, the EGFR inhibitor is any of embodiments as described in Section III-1: PTPN11 Inhibitors and/or EGFR Inhibitors.

In some embodiments, the EGFR inhibitor is erlotinib, cetuximab, panitumumab, vandetanib, afatinib, gefitinib, osimertinib, necitumumab, brigatinib, neratinib, dacomitinib, amivantamab (JNJ-61186372), mobocertinib (TAK-788), BLU-945, varlitinib, tarloxitinib, poziotinib, or lapatinib. In some embodiments, the EGFR inhibitor is osimertinib. In some embodiments, the EGFR inhibitor is erlotinib.

The compositions of the present disclosure (including the compound of formula (I) and the EGFR inhibitor) can be prepared in a wide variety of oral, parenteral and topical dosage forms. Oral preparations include tablets, pills, powder, dragees, capsules, liquids, lozenges, cachets, gels, syrups, slurries, suspensions, etc., suitable for ingestion by the patient. The compositions of the present disclosure can also be administered by injection, that is, intravenously, intramuscularly, intracutaneously, subcutaneously, intraduodenally, or intraperitoneally. Also, the compositions described herein can be administered by inhalation, for example, intranasally. Additionally, the compositions of the present disclosure can be administered transdermally. The compositions of this disclosure can also be administered by intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995).

For preparing pharmaceutical compositions of the present disclosure (including the compound of formula (I) and the EGFR inhibitor), pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton PA (“Remington's”).

In powders, the carrier is a finely divided solid, which is in a mixture with the finely divided active component. In tablets, the active components are mixed with the carrier having the necessary binding properties in suitable proportions and compacted in a particular shape and size.

The powders, capsules and tablets preferably contain from about 5% to about 70% of the active compounds, such as from about 10% to about 70% of the active compounds (e.g., the compound of formula (I) and the EGFR inhibitor). Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other excipients, is surrounded by a carrier, which is thus in association with it. Similarly, cachets and lozenges are included. Tablets, powders, capsules, pills, cachets, and lozenges can be used as solid dosage forms suitable for oral administration.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins including, but not limited to, gelatin and collagen. Disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the present disclosure can also be used orally using, for example, push-fit capsules made of gelatin, as well as soft, sealed capsules made of gelatin and a coating such as glycerol or sorbitol. Push-fit capsules can contain the active compounds (e.g., the compound of formula (I) and the EGFR inhibitor) mixed with a filler or binders such as lactose or starches, lubricants such as talc or magnesium stearate, and, optionally, stabilizers. In soft capsules, the active compounds (e.g., the compound of Formula (I) and the EGFR inhibitor) may be dissolved or suspended in suitable liquids, such as fatty oils, liquid paraffin, or liquid polyethylene glycol with or without stabilizers.

For preparing suppositories, a low melting wax, such as a mixture of fatty acid glycerides or cocoa butter, is first melted and the active compounds (e.g., the compound of formula (I) and the EGFR inhibitor) are dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active compounds (e.g., the compound of formula (I) and the EGFR inhibitor), as defined and described herein, in water and adding optional suitable colorants, flavors, stabilizers, and thickening agents. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending the active compounds (e.g., the compound of formula (I) and the EGFR inhibitor) in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the present disclosure can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The compositions of the present disclosure (including the compound of formula (I) and the EGFR inhibitor) can be delivered by any suitable means, including oral, parenteral and topical methods. Transdermal administration methods, by a topical route, can be formulated as applicator sticks, solutions, suspensions, emulsions, gels, creams, ointments, pastes, jellies, paints, powders, and aerosols.

The compositions of the present disclosure (including the compound of formula (I) and the EGFR inhibitor) can also be delivered as microspheres for slow release in the body. For example, microspheres can be formulated for administration via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

In another embodiment, the compositions of the present disclosure can be formulated for parenteral administration, such as intravenous (IV) administration or administration into a body cavity or lumen of an organ. The formulations for administration will commonly comprise a solution of the compositions of the present disclosure dissolved in a pharmaceutically acceptable carrier. Among the acceptable vehicles and solvents that can be employed are water and Ringer's solution, an isotonic sodium chloride. In addition, sterile fixed oils can be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by various sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the compositions of the present disclosure in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In another embodiment, the formulations of the compositions of the present disclosure can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the compositions of the present disclosure into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

Lipid-based drug delivery systems include lipid solutions, lipid emulsions, lipid dispersions, self-emulsifying drug delivery systems (SEDDS) and self-microemulsifying drug delivery systems (SMEDDS). In particular, SEDDS and SMEDDS are isotropic mixtures of lipids, surfactants and co-surfactants that can disperse spontaneously in aqueous media and form fine emulsions (SEDDS) or microemulsions (SMEDDS). Lipids useful in the formulations of the present disclosure include any natural or synthetic lipids including, but not limited to, sesame seed oil, olive oil, castor oil, peanut oil, fatty acid esters, glycerol esters, Labrafil®, Labrasol®, Cremophor®, Solutol®, Tween®, Capryol®, Capmul®, Captex®, and Peceol®.

The pharmaceutical formulations of the present disclosure can be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preparation may be a lyophilized powder in, e.g., 1 mM-50 mM histidine, 0.1%-2% sucrose, 2%-7% mannitol at a pH range of 4.5 to 5.5, that is combined with buffer prior to use.

The pharmaceutical formulations of the present disclosure can be provided as a salt and can be formed with bases, namely cationic salts such as alkali and alkaline earth metal salts, such as sodium, lithium, potassium, calcium, magnesium, as well as ammonium salts, such as ammonium, trimethyl-ammonium, diethylammonium, and tris-(hydroxymethyl)-methyl-ammonium salts.

### VI. Kits

In another aspect, the present disclosure provides a kit for treating a disease or disorder (e.g., cancer) in a subject, the kit including:
a) a therapeutically effective amount of a PTPN11 inhibitor; and
b) a therapeutically effective amount of an EGFR inhibitor,
   together with instruction for effective administration,
      wherein the PTPN11 inhibitor is represented by formula (I) as defined and described herein.

The cancer and/or solid tumor are described according to Section III-2: Cancer/Solid Tumor. In some embodiments, the cancer and/or solid tumor are any of embodiments as described in Section III-2: Cancer/Solid Tumor.

The subject is described according to Section III-3: Subject. In some embodiments, the subject is any of embodiments as described in Section III-3: Subject.

The PTPN11 inhibitor represented by formula (I) is described according to Section III-1: PTPN11 Inhibitors and/or EGFR Inhibitors. In some embodiments, the PTPN11 inhibitor of formula (I) is any of embodiments as described in Section III-1: PTPN11 inhibitors and/or EGFR Inhibitors. In some embodiments, the PTPN11 inhibitor of formula (I) is the compound of formula (10b).

The PTPN11 inhibitor of formula (I) is further described according to Section IV. Compounds. In some embodiments, the PTPN11 inhibitor of formula (I) is any of embodiments as described in Section IV. Compounds.

The EGFR inhibitor is described according to Section III-1: PTPN11 Inhibitors and/or EGFR Inhibitors. In some embodiments, the EGFR inhibitor is any of embodiments as described in Section III-1: PTPN11 Inhibitors and/or EGFR Inhibitors.

In some embodiments, the EGFR inhibitor is erlotinib, cetuximab, panitumumab, vandetanib, afatinib, gefitinib, osimertinib, necitumumab, brigatinib, neratinib, dacomitinib, amivantamab (JNJ-61186372), mobocertinib (TAK-788), BLU-945, varlitinib, tarloxitinib, poziotinib, or lapatinib. In some embodiments, the EGFR inhibitor is osimertinib. In some embodiments, the EGFR inhibitor is erlotinib.

In some embodiments, the kit includes instructions for administration of the compound of formula (I) or (10b) and the EGFR inhibitor. In some embodiments, the kit includes instructions for administration of the compound of formula (10b) and the EGFR inhibitor. In some embodiments, such instructions include directions relating to safety provisions as well as timing and amounts of administration of the compound of formula (I) or (10b) and the EGFR inhibitor. In some embodiments, such instructions include directions relating to safety provisions as well as timing and amounts of administration of the compound of formula (10b) and the EGFR inhibitor.

The PTPN11 inhibitor represented by formula (I) or (10b) as described herein and the EGFR inhibitor as described herein can be are formulated for concomitant administration or sequential administration. In some embodiments, the PTPN11 inhibitor of formula (I) or (10b) and the EGFR inhibitor are formulated for concomitant administration. In some embodiments, the PTPN11 inhibitor of formula (I) or (10b) and the EGFR inhibitor are formulated for sequential administration. In some embodiments, the PTPN11 inhibitor of formula (I) or (10b) is administered prior to the administration of the EGFR inhibitor. In some embodiments, the PTPN11 inhibitor of formula (I) or (10b) is administered after the administration of the EGFR inhibitor.

### VII. List of Abbreviations

| Abbreviation | Definition |
|---|---|
| ASPA | Aminals Scientific Procedures Act 1986 |
| ATCC | American Type Culture Collection |
| Axl | AXL receptor tyrosine kinase |
| BQL | Below Quantitation Limit |
| BW | Body Weight |
| BWL | Body Weight Loss |
| cDNA | complementary Deoxyribonucleic acid |
| Cₘₐₓ | Maximum blood plasma concentration |
| c-Met | Tyrosine-protein kinase Met |
| ddH2O | double-distilled water |
| DMSO | Dimethyl sulfoxide |
| DPBS | Dulbecco's phosphate buffered saline |
| DUSP | Dual Specificity Phosphatase |
| EDTA | Ethylenediaminetetraacetic acid |
| EGR1 | Early Growth Response 1 |
| ER | Erlotinib resistant |
| ERK | Extracellular signal-regulated kinase |
| ETV | ETS Variant Transcription Factor |
| FAM | Fluorescein amidites |
| FBS | fetal bovine serum |
| FC | Fold change |
| FGFR1 | Fibroblast Growth Factor Receptor 1 |
| FOSL1 | FOS Like 1, AP-1 Transcription Factor Subunit |
| HPMC | Hydroxypropyl methylcellulose |
| IC₅₀ | Half maximal inhibitory concentration |
| IGF-1Rβ | Insulin Like Growth Factor 1 Receptor b |
| ISO | International Organization for Standardization |
| LC-MS/MS | Liquid chromatography-mass spectrometry/mass spectrometry |
| MPAS | MAPK pathway activity score |
| mRNA | Messenger ribonucleic acid |
| NACWO | Named Animal Care and Welfare Officer |
| NVS | Named Veterinary Surgeon |
| OsiR | Osimertinib resistant |
| PBS | Phosphate-buffered saline |
| PDGFR | Platelet Derived Growth Factor Receptor |
| PO | Per os (oral administration) |
| qRT-PCR | Quantitative real time polymerase chain reaction |
| RNA | Ribonucleic acid |
| RPLP0 | Ribosomal Protein, Large, P0 |
| RPM | Round per minute |
| RPMI | Roswell Park Memorial Institute |
| RTK | Receptor tyrosine kinase |
| SD (in Exmples 2 and 3) | Standard Deviation |
| SEM | Standard error of the mean |
| subQ | Subcutaneous |
| T790M | Threonine 790 to Methionine mutation |
| TGI | Tumor Growth Inhibition |
| (e)CRF | (electronic) case report form |
| (p)ERK | (phosphorylated) extracellular signal-regulated kinase |
| AE | adverse event |
| ALK | anaplastic lymphoma kinase |
| ALT | alanine aminotransferase (or transaminase) |
| ANC | absolute neutrophil count |
| aPTT | activated partial thromboplastin time |
| AST | aspartate aminotransferase (or transaminase) |
| AUC | area under the plasma concentration-time curve |
| BBP | BridgeBio Pharma |
| BCRP | breast cancer resistance protein |
| BICR | blinded independent central review |
| BOIN | Bayesian optimal interval |
| CI | confidence interval |
| CLIA | Clinical Laboratory Improvement Amendments |
| Cₘₐₓ | maximum plasma drug concentration |
| CNS | central nervous system |
| CR | complete response(s) |
| CRO | contract research organization |
| CT | computed tomography |
| CTCAE | Common Terminology Criteria for Adverse Events |
| ctDNA | circulating tumor deoxyribonucleic acid |
| CYP | cytochrome P450 |
| DLT | dose-limiting toxicity |
| DNA | deoxyribonucleic acid |
| DOR | duration of response |
| DUSP6 | dual specificity phosphatase 6 |
| ECG | electrocardiogram |
| ECHO | echocardiogram |
| ECOG | Eastern Cooperative Oncology Group |
| EDC | electronic data capture (system) |
| EGFR | epidermal growth factor receptor |
| EORTC | European Organization for Research and Treatment of Cancer |
| EOS | end of study |
| EOT | end of treatment |
| FDA | Food and Drug Administration |
| FSH | follicle-stimulating hormone |
| GCP | Good Clinical Practice |
| GLP | Good Laboratory Practice |
| GMP | Good Manufacturing Practice |
| GTP | guanosine triphosphate |
| HBV | hepatitis B virus |
| HCV | hepatitis C virus |
| HIV | human immunodeficiency virus |
| HNSTD | highest non-severely toxic dose |
| IACS | Institute for Applied Cancer Science |
| IB | Investigator's Brochure |
| ICF | informed consent form |
| ICH | International Conference on Harmonization |
| IEC | Independent Ethics Committee |
| INR | international normalized ratio |
| IRB | Institutional Review Board |
| IV | intravenous |
| KRAS | Kirsten rat sarcoma viral oncogene homolog |
| LC-MS/MS | liquid chromatography with tandem mass spectrometry |
| LD (or LDi) | longest diameter |
| LVEF | left ventricular ejection fraction |
| MAPK | mitogen-activated protein kinase |
| MATE | multidrug and toxin extrusion protein |
| MRI | magnetic resonance imaging |
| MTD | maximum tolerated dose |
| MUGA | multiple-gated acquisition scan (radionuclide angiography) |
| NCI | National Cancer Institute |
| NGS | next generation sequencing |
| NSCLC | non-small cell lung cancer |
| OATP | organic anion transporter polypeptide |
| ORR | objective response rate |
| OS | overall survival |
| PD | progressive disease |
| PD-1 | programmed cell death-1 |
| PD-L1 | programmed cell death ligand-1 |
| PET | positron emission tomography |
| PFS | progression free survival |
| P-gp | P-glycoprotein |
| PI | package insert |
| PK | pharmacokinetic |
| PR | partial response |
| PS | performance status |
| PT | prothrombin time |
| PTPN11 | protein tyrosine phosphatase non-receptor type 11, the gene encoding |
| | SHP2 |
| QD | once daily |
| RAF | rapidly accelerated fibrosarcoma |
| RAS | any of a family of genes that undergo mutation to oncogenes and |
| | especially to some commonly linked to human cancers (as of the |
| | colon, lung, and pancreas) |
| REB | Research Ethics Board |
| RECIST | Response Evaluation Criteria in Solid Tumors |
| RNA | ribonucleic acid |
| RP1bD | recommended phase 1b dose |
| RP2D | recommended phase 2 dose |
| RTK | receptor tyrosine kinase |
| SAE | serious adverse event |
| SAP | Statistical Analysis Plan |
| SD | stable disease |
| SGOT | serum glutamic-oxaloacetic transaminase |
| SGPT | serum glutamic-pyruvic transaminase |
| SHP2 | Src homology-2-containing protein tyrosine phosphatase 2 |
| SRC | Safety Review Committee |
| STD10 | severely toxic dose in 10% of animals |
| SUSAR | suspected unexpected serious adverse reaction |
| TEAE | treatment-emergent adverse event |
| Tₘₐₓ | time to reach maximum drug concentration following drug |
| | administration |
| ULN | upper limit of normal |
| US | United States |

### VIII. Examples

### Example 1: Phenotypic and Pharmacodynamics Response in HCC4006 Cell Lines

### A. Materials

### Test Article #1—Formula (10b):

Chemical name: 6-[(3S,4S)-4-amino-3-methyl-2-oxa-8-azaspiro[4.5]decan-8-yl]-3-(2,3-dichlorophenyl)-2,5-dimethyl-3,4-dihydropyrimidin-4-one
Molecular formula: C₂₁H₂₆Cl₂N₄O₂
Molecular weight: 437.37

### Test Article #2—Osimertinib (Selleck Chem #S7297):

CAS #: 1421373-65-0
Molecular weight: 499.61

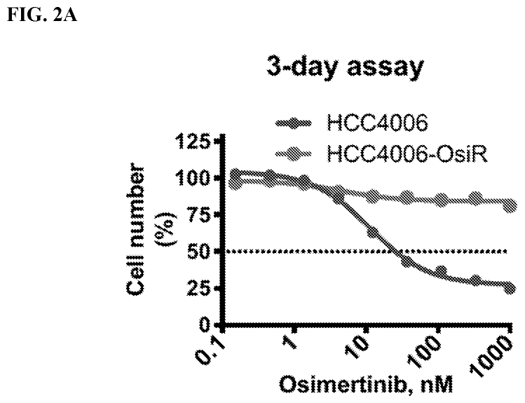

Cell lines. The HCC4006 cell line was purchased from ATCC. It is a non-small cell lung cancer (NSCLC) cell line harboring an EGFR activating mutation (EGFR exon 19 deletion), and has been demonstrated to be sensitive to the third generation EGFR inhibitor osimertinib in vitro. The cells were cultured in RPMI-1640 containing 4 mM glutamax (Thermo Fisher #72400-120)+10% fetal bovine serum (FBS, Sigma #F2442) in 37° C. tissue culture incubator with 5% CO₂ (NUAIRE, NU-5510). The HCC4006-OsiR (Osimertinib Resistant) cell line was derived from HCC4006 cells by culturing in the presence of 1 M osimertinib (Selleck Chem #S7279). The cells were maintained in RPMI-1640 containing 4 mM glutamax (Thermo Fisher #72400-120)+10% fetal bovine serum (FBS, Sigma #F2442)+1 μM osimertinib. The HCC4006-OsiR cells demonstrated a 40-fold shift in osimertinib IC₅₀ compared with the HCC4006 parental cells in a 3-day proliferation assay in vitro (FIG. 2A). Genomic characterizations have demonstrated that the HCC4006-OsiR model maintains the same EGFR activating mutation as the parental model (i.e., exon 19 deletion), does not harbor EGFR T790M mutation, and has increased expression of multiple RTKs including AXL, FGFR1, PDGFR and IGF-1Rβ. The HCC4006-OsiR cells also demonstrate increased mesenchymal features and reduced epithelial features.

### B. Experimental Procedures

In vitro 3-day proliferation assay. HCC4006 or HCC4006-OsiR cells were plated in 96-well plates (Corning #3603) at 1,500 cells per well in 100 μL of media (RPMI-1640+10% FBS) and allowed to attach overnight. The next day, the cells were switched to 100 μL media containing DMSO or serially diluted osimertinib (9-point titration: 0.15 nM˜1,000 nM final concentration). Final DMSO concentration was 0.1%. After 3 days of treatment, viable cell numbers were determined using CellTiter-Glo 2.0 cell viability assay (Promega #G9241).

CellTiter-Glo 2.0 cell viability assay. CellTiter-Glo 2.0 cell viability assay (Promega #G9241) was used to determine the relative viable cell number, following manufacturer's protocols. Briefly, 100 μL CellTiter-Glo reagent was added to each well in the 96-well plate. The plate was put on a VWR advanced 3500 orbital shaker (VWR #89032-096) at 100 rpm shaking for 5 min at room temperature, then incubated at room temperature for 15 min, protected from the light. Afterwards, luminescence was quantified using a PheraStar plate reader (BMG Labtech). The luminescence readings were normalized to the average of DMSO treated samples of each cell line, using Microsoft Excel. Data were plotted in GraphPad Prism 8.0. Non-linear fit was used to generate the dose response curve. IC₅₀ refers to the compound concentration at which quantified cell number was 50% of DMSO-treated cells. IC₅₀ was interpolated using GraphPad Prism 8.0.

In vitro clonogenic assay. HCC4006 or HCC4006-OsiR cells were plated in 24-well plates (Corning #353047) at 800 cells (HCC4006) or 1000 cells (HCC4006-OsiR) per well in 0.5 mL of media (RPMI-1640+10% FBS) and allowed to attach overnight. The next day, 0.25 mL media containing DMSO or serially diluted formula (10b) (3-point titration: 0.37 μM, 1.1 μM or 3.3 M final concentration) was added to each well. Immediately afterwards, 0.25 mL media containing DMSO or serially diluted osimertinib (5-point titration: 0.46 nM˜37 nM for HCC4006 cells, and 4.1 nM˜333 nM for HCC4006-OsiR cells) was added to each well. Compounds were prediluted in media at 4x the final concentration, and the final DMSO concentration was 0.2% in 1 mL of media per 24-well. After 12˜14 days of treatment, the plates were stained with crystal violet solution.

Crystal violet staining, quantification and Bliss score calculation. The 0.1% crystal violet staining solution was prepared by mixing 100 mL of 1% crystal violet solution (Sigma #V5265), 100 mL of 100% ethanol and 800 mL of ddH₂O. Media was removed from cell culture wells, and 1 mL of sterile Dulbecco's phosphate buffered saline (DPBS, Life technologies #14190-144) was gently added to each well. After removal of DPBS, 0.5 mL of 0.1% crystal violet staining solution was added to each 24-well. The plates were incubated at room temperature for 30 min. The staining solution was removed from wells and discarded. Each well was then washed with 1 mL of ddH₂O. Afterwards, the plates were dipped in a large bucket filled with tap water 2˜4 times until the supernatant became colorless. Remaining liquid was shaken out and the plates left upside down, uncovered, for overnight to dry. Once completely dry, the plates were scanned on an image scanner (EPSON Perfection V700) at 300 dpi.

To quantify the signal, 0.5 mL of 10% acetic acid (Fisher #BP2401-212) was added to each well of the dried plates. The plates were put on a VWR advanced 3500 orbital shaker (VWR #89032-096) at 100 rpm shaking for 30 min at room temperature, to solubilize the crystal violet. Afterwards, 100 μL solution from each well was transferred to a clear 96-well plate (Spectra Plate 96 MB, PerkinElmer #6005640) and the absorbance at 590 nm was read on the PheraStar plate reader (BMG Labtech). If the signal was saturated, the solutions were further diluted with equal volumes of 10% acetic acid, till OD590 was below 3.5.

The OD590 readings (multiplied by the dilution factor) were normalized to the average of DMSO treated samples, using Microsoft Excel. The Bliss expectation was calculated with the equation (A+B)−A×B/100, where A and B were the percentage growth inhibition induced by agents A and B at a given dose. Bliss score is the difference between the detected growth inhibition (%) caused by the combination of the two agents and the bliss expectation. Positive Bliss scores indicate combinations where the effects are synergistic. The heatmap for Bliss score was generated using Prism GraphPad 8.0.

In vitro pharmacodynamic assay—Taqman qRT-PCR assay. The HCC4006-OsiR cells were plated in 6-well plates (Corning #353046) in 2 mL media and allowed to adhere overnight. Plating density was adjusted according to treatments and length of the experiment (Table 1). The day after plating (Day 1), cells were treated by adding to each well 1 mL of media containing DMSO or 12 μM formula (10b), and 1 mL of media containing DMSO or 400 nM osimertinib. Four hours later, 4-hour samples were harvested by removing media and lysing cells in each well with 350 μL RLT lysis buffer (Qiagen #79216) with 1% 2-mercaptoethanol. Samples were processed either right way or stored at −20° C. until further analysis. On day 3 and day 8, 48-hour samples and 7-day samples were harvested in the same way. Cells harvested at these two timepoints were switched to fresh media containing compounds 24 hours before sample harvesting.

TABLE 1
Plating Density vs. Treatments and Length of the Experiment

| | Plating density (cells/well) | | |
|---|---|---|---|
| Treatments | 4-hour | 48-hour | 7-day |
| DMSO | 200,000 | 30,000 | 10,000 |
| Formula (10b), 3 μM | 200,000 | 60,000 | 20,000 |
| Osimertinib, 100 nM | 200,000 | 30,000 | 10,000 |
| Formula (10b), 3 μM + Osimertinib, 100 nM | 200,000 | 90,000 | 30,000 |

RNA was extracted from lysates using RNeasy mini RNA isolation kit (Qiagen #74106). Isolated RNA was quantified using Nanodrop 8000 (Thermo Fisher). cDNA was synthesized using high capacity cDNA reverse transcription kit (Thermo Fisher #4368813) with 1 μg total RNA per reaction on a BioRad Tetrad2 thermal cycler (25° C. for 10 min, 37° C. for 120 min, 85° C. for 5 min then hold at 4° C.). The cDNA was further diluted 1:10 using ddH2O. Quantitative real-time PCR was conducted using the following Taqman probes and Taqman universal master mix II without UNG (Thermo Fisher #4440040) on QuantStudio 6 in 384-well format (Thermo Fisher), following vendor's protocols. Each well contains a target probe of interest conjugated to FAM dye (DUSP6, Thermo Fisher Assay ID Hs01044001_m1), and an endogenous control conjugated to VIC dye (human RPLPO, Thermo Fisher #4326314E). Each cDNA was run in three technical replicates.

The ΔΔCt method was used to analyze the qRT-PCR data. Relative mRNA levels were calculated using the formula below. To calculate fold change, the relative mRNA levels of each sample were normalized to that in DMSO treated samples (fold change for DMSO treated samples was set at “1”). Calculations were done in Microsoft Excel and the graphs were generated in GraphPad Prism 8.0.

$$Relative mRNA levels = 2 \hat{} ( {C}_{t of control gene} - {C}_{t of gene of interest} )$$

### C. Results

The EGFR tyrosine kinase inhibitor osimertinib is currently approved in the US for the first-line treatment of patients with metastatic NSCLC whose tumors harbor an activating mutation in EGFR. Patients on osimertinib treatment will ultimately experience disease progression, with acquired resistance being a major clinical challenge. Activation of alternate RTKs (“RTK bypass”) to maintain MAPK pathway signaling downstream of RTK is a clinically observed resistance mechanism towards both first-generation and third-generation EGFR inhibitors. Since SHP2 inhibition impacts the MAPK pathway downstream of multiple RTKs, it is hypothesized that a SHP2 inhibitor such as formula (10b) might potentiate the activity of osimertinib and may also have activity in osimertinib-resistant tumors. The experiments below were designed to evaluate the in vitro activity of the SHP2 inhibitor formula (10b) in combination with osimertinib in both an osimertinib-sensitive model and an osimertinib resistant model with “RTK bypass” resistance mechanism.

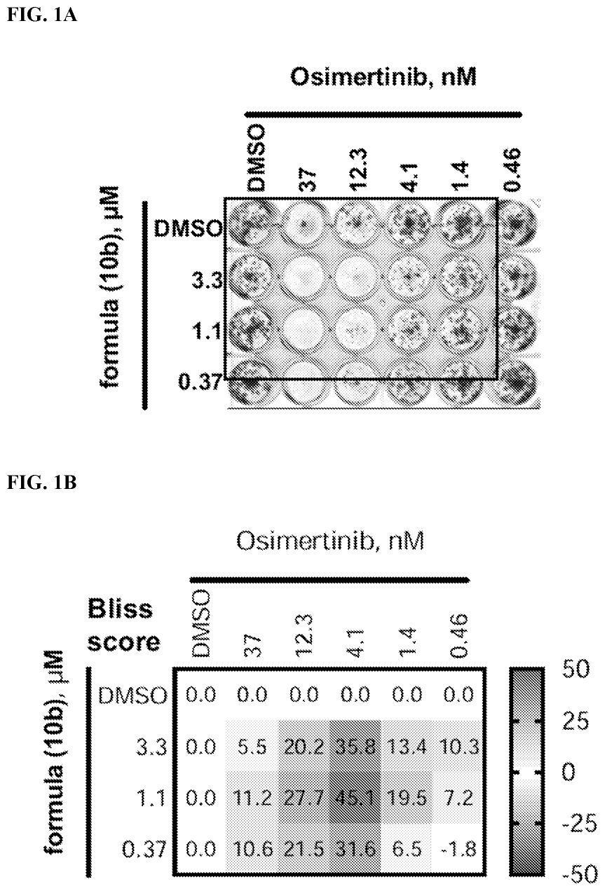

Treatment with the combination of formula (10b) and osimertinib synergistically suppresses the proliferation of osimertinib-sensitive HCC4006 cells in vitro. In an in vitro clonogenic assay, the combination of osimertinib and formula (10b) suppressed the proliferation of the osimertinib-sensitive HCC4006 human tumor cell lines, in a dose-dependent manner (FIG. 1A). For most of the concentrations tested, treatment with the combination of formula (10b) and osimertinib more potently suppressed cell proliferation than treatment with each single agent at the same concentration. Bliss score calculation indicated a bliss score>0 for most of the concentrations tested, suggesting a synergistic effect between osimertinib and formula (10b) in suppressing the proliferation of HCC4006 cells in vitro (FIG. 1B).

HCC4006-OsiR model demonstrates acquired resistance towards osimertinib in vitro. In order to evaluate the activity of formula (10b) in a cell line model with acquired resistance to osimertinib, the HCC4006-OsiR cell line was generated by culturing EGFRₘᵤₜ HCC4006 cells in the presence of 1 μM osimertinib for ˜3 months, till HCC4006-OsiR cells were proliferating at a similar rate as the parental cells. In a 3-day proliferation assay (FIG. 2A), as expected, the HCC4006 parental cells were sensitive to osimertinib, with IC₅₀ at 27.0±7.9 nM. In contrast, the HCC4006-OsiR cells were resistant to osimertinib, with 1,000 nM (the highest concentration tested) having no anti-proliferative effect in this assay. Thus, the osimertinib IC₅₀ in HCC4006-OsiR cells in a 3-day in vitro proliferation assay was above 1,000 nM, which was approximately at least 40-fold higher compared with that in the HCC4006 parental cells. Furthermore, in a 14-day clonogenic assay, HCC4006-OsiR cells also demonstrated significant resistance towards osimertinib with a 20-fold shift in IC₅₀ compared with the HCC4006 parental cells (IC₅₀ in HCC4006 is 12.2±2.5 nM, and in HCC4006-OsiR 257±241 nM) (FIG. 2B).

Genomic characterizations demonstrated that the HCC4006-OsiR model maintained the same EGFR activating mutation as the parental model (i.e., exon 19 deletion), did not harbor EGFR T790M mutation, and had increased expression of multiple RTKs including AXL, FGFR1, PDGFR and IGF-1Rb. The HCC4006-OsiR cells also demonstrate increased mesenchymal features and reduced epithelial features.

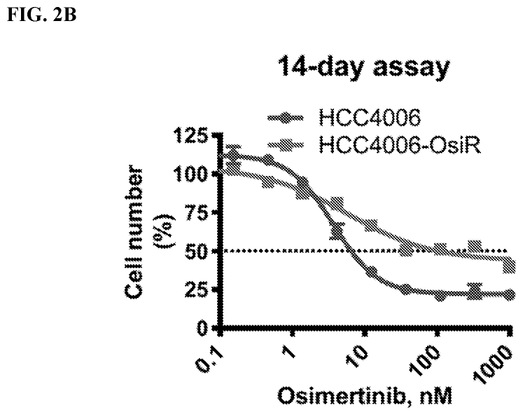

Treatment with the combination of formula (10b) and osimertinib synergistically suppresses the proliferation of osimertinib-resistant HCC4006-OsiR cells in vitro. In an in vitro clonogenic assay, the combination of osimertinib and formula (10b) suppressed the proliferation of the osimertinib-resistant HCC4006-OsiR human tumor cell line, in a dose dependent manner (FIG. 3A). For most of the concentrations tested, treatment with the combination of formula (10b) and osimertinib more potently suppressed cell proliferation than treatment with each single agent at the same concentration. Bliss score calculation indicated a bliss score>0 in most of the concentrations tested, suggesting a synergistic effect between osimertinib and formula (10b) in suppressing the proliferation of HCC4006-OsiR cells in vitro (FIG. 3B).

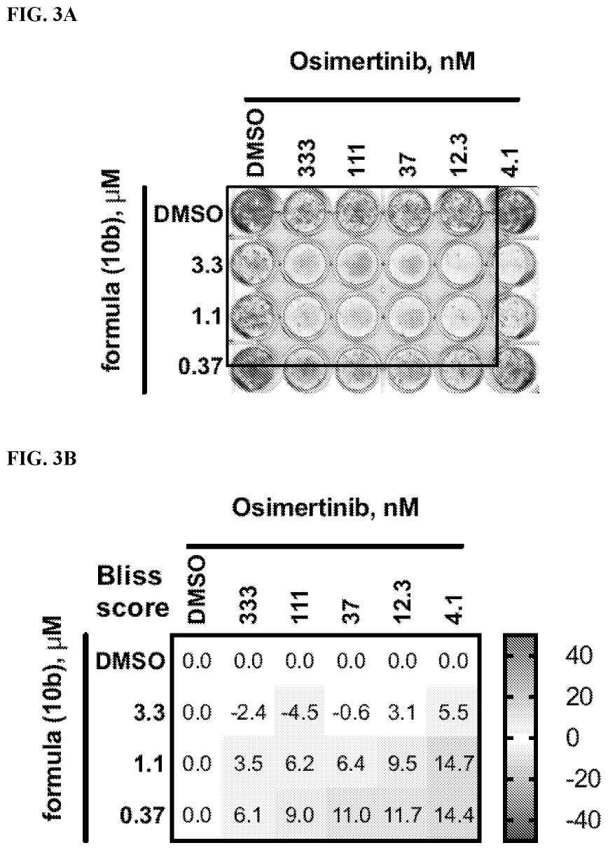

Formula (10b) as a single agent or in combination with osimertinib suppresses MAPK pathway signaling in osimertinib-resistant HCC4006-OsiR cells in vitro. Transcript levels of MAPK signature genes such as DUSP6 can be used as a readout of MAPK pathway activity. In the osimertinib-resistant HCC4006-OsiR model, osimertinib 100 nM (˜8× the in vitro IC₅₀ of osimertinib in 14-day clonogenic assay in the HCC4006 parental line) did not suppress DUSP6 mRNA levels with 4-hour, 48-hour or 7-day treatment, consistent with this model being osimertinib-resistant (FIG. 4). In contrast, formula (10b) potently suppressed DUSP6 mRNA levels at all three timepoints tested, with the suppression being most robust following 4-hour treatment; DUSP6 transcript levels partially recovered following prolonged treatment with formula (10b) (i.e., 48-hour or 7-day treatment), presumably due to the release of the negative feedback of pERK/pMEK on upstream signaling factors. Treatment with the combination of formula (10b) 3 μM and osimertinib 100 nM had a similar impact on DUSP6 mRNA levels as treatment with formula (10b) single agent at 3 μM (FIG. 4). Treatment with formula (10b) at lower concentrations (1 μM and 0.5 μM) as a single agent or in combination with 100 nM osimertinib were also tested, and the data also suggest the combination treatment had similar impact on DUSP6 mRNA levels as formula (10b) single agent. This is distinct from the observations in proliferation assays where the combination treatment more potently suppressed cell proliferation than formula (10b) single agent (FIG. 3A). Therefore, it is likely that there are additional mechanisms underlying the combination effect between formula (10b) and osimertinib on the proliferation of HCC4006-OsiR cells in vitro. In summary, these data suggest formula (10b) as a single agent or in combination with osimertinib suppresses MAPK pathway signaling in the osimertinib-resistant HCC4006-OsiR cells in vitro.

### D. Conclusions

The combination of formula (10b) and osimertinib treatment synergistically suppresses the proliferation of both osimertinib-sensitive HCC4006 and osimertinib-resistant HCC4006-OsiR human tumor cell lines in vitro. In addition, formula (10b) as a single agent or in combination with osimertinib potently suppresses MAPK pathway signaling (as measured by DUSP6 transcript levels) in HCC4006-OsiR cells in vitro.

### Example 2: In Vivo Anti-Tumor Efficacy of Formula (10b) in Combination with Osimertinib

### A. Materials

### Test Article #1—Formula (10b):

Chemical name: 6-[(3S,4S)-4-amino-3-methyl-2-oxa-8-azaspiro[4.5]decan-8-yl]-3-(2,3-dichlorophenyl)-2,5-dimethyl-3,4-dihydropyrimidin-4-one
Molecular formula: C₂₁H₂₆Cl₂N₄O₂
Molecular weight: 437.37

### Test Article #2—Osimertinib (ChemieTek #CT-A9291):

CAS #: 1421373-65-0
Molecular weight: 499.61

Cell lines. The HCC827 cell line was purchased from ATCC. It is a non-small cell lung cancer (NSCLC) cell line harboring an EGFR activating mutation (EGFR exon 19 deletion), and has been demonstrated to be sensitive to the third-generation EGFR inhibitor osimertinib in vitro and in vivo. The cells were cultured in RPMI-1640 medium (Sigma #RO883)+10% FBS (Sigma #F7524)+2 mM L-glutamine (Sigma #59202C) in 37° C. tissue culture incubator with 5% CO₂. The HCC827-ER1 (Erlotinib Resistant) cell line was derived from HCC827 cells at Crown Bioscience UK Ltd. The resistant cells were generated by culturing in the presence of escalating concentrations of erlotinib. The cells were maintained in RPMI-1640 medium (Sigma #RO883)+10% FBS (Sigma #F7524)+2 mM L-glutamine (Sigma #59202C)+42 μM erlotinib (LC Laboratories #E-4007). The HCC827-ER1 cells demonstrated 10,000-fold shift in erlotinib IC₅₀ in vitro compared with the HCC827 parental cells (unpublished data from Crown Bioscience UK Ltd). Genomic characterizations have demonstrated that the HCC827-ER1 model maintains the same EGFR activating mutation as the parental model (i.e., exon 19 deletion), does not harbor EGFR T790M mutation, and harbors c-MET amplification as a resistance mechanism.

Test animals. Female athymic nude mice (Envigo UK, Hsd: Athymic Nude-Foxn1nu) were utilized in this experiment. Animals were between 5 and 6 weeks of age at time of xenograft implantation. Animal welfare complies with the UK Animals Scientific Procedures Act 1986 (ASPA) in line with Directive 2010/63/EU of the European Parliament and the Council of 22 Sep. 2010 on the protection of animals used for scientific purposes. All experimental data management and reporting procedures were in strict accordance with applicable Crown Bioscience UK Guidelines and Standard Operating Procedures.

All mice were housed in ISO 9001:2015 accredited animal research facilities on the Crown Bioscience UK Ltd site at Hillcrest, Dodgeford Lane, Loughborough, UK. All animals were maintained under the supervision and care of the NVS (Named Veterinary Surgeon) and NACWO (Named Animal Care and Welfare Officer) of the Animal Facilities, who oversee a comprehensive and well-executed health surveillance program. Veterinary medical services are provided on a full-time basis, and veterinarians and clinical staff are on-call for emergencies and/or special situations 24 hours a day, 7 days a week. Mice were housed in cages with a maximum of five animals and had free access to food and water with light 12-hour on/12-hour off. All animals received Teklad 2919 diet and water ad libitum. Mice were monitored daily and cages changed once every second week.

### B. Experimental Procedures

Formulation. Formula (10b) was prepared in 0.5% methylcellulose. To prepare 0.5% methylcellulose solution, methylcellulose powder (400 cP, Sigma #M0262) was added to heated 80° C. sterile water. The solution was incubated at 80° C. with stirring for 3˜4 hours, then incubated at 4° C. with continuous stirring for 18 hours. After adjusting the final volume with sterile H₂O, the solution as stirred for another 30 min at 4° C., then filtered using 0.45 μM sterile filter. The prepared 0.5% methylcellulose solution was stored at 4° C. for future use.

To prepare formula (10b) dosing suspension in 0.5% methylcellulose, the weighed compound was placed in a glass vial and 0.5% methylcellulose solution was added to the vial with a syringe. After vortexing for 30 seconds, the vial was sonicated in a water bath sonicator (Fisher Scientific Ultrasonic Bath 5.7L Model 3800, #15337418) on “High” setting at room temperature for ˜ 20 min until an off-white suspension without major white solids was obtained. The prepared dosing suspension was stored at 4° C. with gentle continuous stirring. Fresh dosing suspension was prepared once a week.

Osimertinib was prepared in 0.5% Hydroxypropyl methylcellulose (HPMC). To prepare 0.5% HPMC solution, 0.5 g of HPMC powder (Sigma #H9262) was added to approximately 80 mL of sterile water in a glass beaker, and mixed on a magnetic stir plate for ˜1 hour. Once all powder dissolved, the solution was transferred to a 100 mL volumetric flask. The beaker was rinsed with sterile water and the final volume was adjusted to 100 mL using the rinse. The prepared 0.5% HPMC solution was stored at 4° C. for future use.

To prepare osimertinib dosing suspension in 0.5% HPMC, the weighed compound was placed in a glass vial and prepared 0.5% HPMC was added. The mixture was left on a magnetic stir plate overnight at room temperature to ensure all particles were well suspended. The prepared dosing suspension was stored at 4° C. with gentle continuous stirring. Fresh dosing suspension was prepared once a week.

In vivo modeling, treatment and data analysis. For xenograft studies with the HCC827 and HCC827-ER1 cells, 5 million cells in 200 μL Matrigel (Corning #354234) were injected subcutaneously into the left flank of 5˜6 weeks old female athymic nude mice (Envigo UK). Tumor size measurement was performed with caliper and calculated using a standard formula: length×width2/2, where length and width were the long and short diameters of the tumor, respectively. When the average volume reached 400 mm³, mice were randomized into groups of 5 mice based on both tumor volume and body weight, and treated with vehicle (0.5% methylcellulose, QD in the morning+0.5% HPMC, QD in the afternoon), osimertinib (0.5% methylcellulose, QD in the morning+osimertinib 5 mg/kg, QD in the afternoon), formula (10b) (formula (10b): 100 mg/kg, QD in the morning+0.5% HPMC, QD in the afternoon), or the combination (formula (10b): 100 mg/kg, QD in the morning+osimertinib 5 mg/kg in the afternoon), through oral gavage (PO). Dosing started ˜3 weeks post the subcutaneous implantation of the tumor cells. Dosing volume was 5 mL/kg for each compound, and was adjusted based on individual mouse weight from the daily measurements. Tumor volume was measured twice a week.

Data were analyzed using Microsoft Excel and GraphPad Prism 8.0. Day 0 was the day before treatment started. Body weight change was calculated based on the following formulas.

$$Body Weight Change \% = ( B {W}_{i} - B {W}_{0} ) / {BW}_{0} * 100 \%$$

BWᵢ and BW₀ are the body weight of an individual mouse on measurement day I and on day 0, respectively.

Blood collection for pharmacokinetic analysis. Blood from live animals for pharmacokinetic (PK) analyses was obtained via the lateral saphenous vein. The mouse was placed in a restraint tube and the hind leg immobilized in the extended position. Resting the foot of the animal on a solid surface, a sterile 25g needle (BD Microlance 3) was inserted into the saphenous vein on the top of the foot. The needle was removed and the blood collected using an EDTA coated capillary blood tube (Microvette CB 300 K2E) by capillary action to a volume of 100 μL. The tube was inverted several times to distribute the EDTA evenly and then centrifuged at 13,000 rpm, 4° C. (VWR Micro Star 17R) for 5 minutes to generate plasma. The supernatant (plasma) was carefully transferred to a 1.5 mL tube and stored at −80° C.

LC-MS/MS quantitation of formula (10b) and osimertinib in mouse plasma. Concentrations of formula (10b) or osimertinib in mouse plasma were quantitated using a validated LC-MS/MS method. For each analysis, 25 μL of plasma sample was precipitated with 200 μL of acetonitrile containing imipramine (70 ng/mL) as an internal standard. This suspension was vortexed for 10 min and centrifuged on a benchtop centrifuge (Eppendorf 5424R) at 4,000 rpm for 10 min, after which 175 μL of the supernatant was transferred to a new tube and diluted with 125 μL of water prior to LC-MS/MS analysis.

LC-MS/MS analysis of formula (10b) or osimertinib was conducted on a Waters Acquity UPLC system coupled with a Waters Xevo TQ-S triple quadrupole mass spectrometer (MS/MS) operated at the positive mode (ESI+). The detection conditions of the mass spectrometer were as follows: capillary voltage, 3 kv; cone voltage, 50 ev; collision energy, 25 ev; source temperature, 150° C.; desolvation temperature, 400° C.; desolvation gas flow, 1000 L/hr; cone gas glow, 0.15 mL/min. Formula (10b) or osimertinib was separated using a SUPELCO Ascentis fused-core C18 column (2.7 μm, 2.1×20 mm) and detected by a multiple reaction monitoring transition (m/z 437.3>186.0 for formula (10b); m/z 500.0>385.1 for osimertinib; m/z 281.1>208.1 for imipramine). The injection volume was 5 μL. The LC mobile phase A was 0.1% acetic acidwater and B was 0.1% acetic acid-acetonitrile. The gradient was 5% B (0-0.3 min), 5-95% B (0.3-1.3 min), 95% B (1.3 to 1.7 min), 95-5% B (1.7-1.71 min), 5% B (1.71 to 2 min) and the flow rate was 0.75 mL/min. The column temperature was 40° C. Under these conditions, the retention time was 1.29 min for formula (10b), 1.27 min for osimertinib, and 1.37 min for the internal standard. The method was validated with the analytical range of 1-1000 ng/ml formula (10b) or osimertinib in untreated CD-1 mouse plasma. All data were processed using the MAssLynx v. 4.0 software.

### C. Results

Osimertinib is currently approved in the US for the first-line treatment of patients with metastatic NSCLC whose tumors harbor an activating mutation in EGFR. Patients on osimertinib treatment will ultimately experience disease progression, with acquired resistance being a major clinical challenge. Activation of alternate RTKs (“RTK bypass”), such as c-MET amplification, to maintain MAPK pathway signaling downstream of RTK is a clinically observed resistance mechanism towards both first-generation and third-generation EGFR inhibitors. Because SHP2 is a critical mediator of RTK signaling, it is hypothesized that osimertinib resistance mediated by “RTK bypass” can be targeted by a SHP2 inhibitor. The experiments below were designed to evaluate the in vivo activity of the SHP2 inhibitor formula (10b) in combination with osimertinib in both an osimertinib-sensitive model and an osimertinib-resistant model with c-MET amplification representing the “RTK bypass” resistance mechanism.

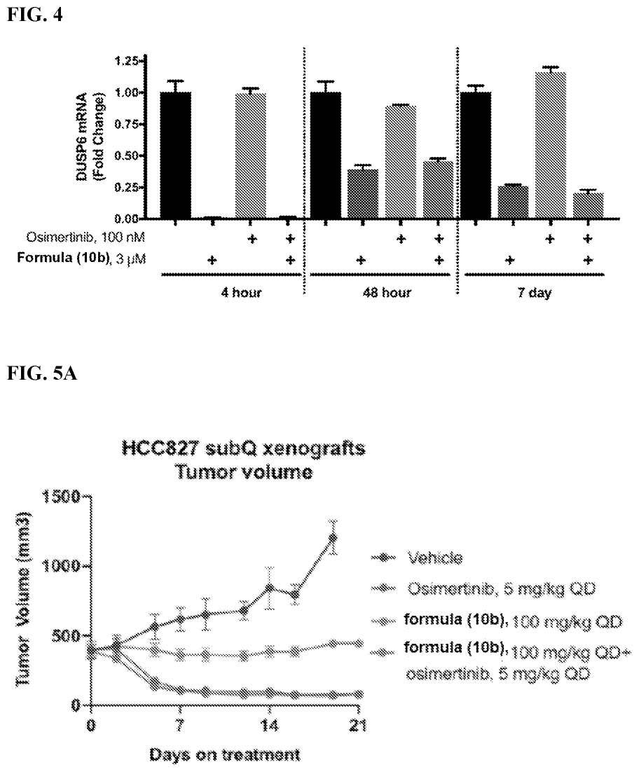

Treatment with formula (10b) single agent or in combination with osimertinib induces tumor growth inhibition in an EGFR^{mut} osimertinib-sensitive subcutaneous model of NSCLC. An osimertinib-sensitive mouse tumor xenograft model was developed by subcutaneous implantation of the HCC827 cells, and was used to test the anti-tumor response of formula (10b) as single-agent treatment, as well as in combination with osimertinib in vivo. Mice harboring established HCC827 subcutaneous tumors (average tumor volume˜400 mm³) were treated with vehicle, osimertinib 5 mg/kg, formula (10b) 100 mg/kg or the combination of the two compounds delivered orally; each compound was administered QD for 21 days. As shown in FIG. 5A, formula (10b) 100 mg/kg QD as a single agent potently suppressed the growth of the HCC827 tumors, leading to tumor stasis. In contrast, osimertinib dosed as a single agent at 5 mg/kg QD caused robust tumor regression. As expected, treatment with the combination of formula (10b) and osimertinib yielded tumor regression, similar to that observed with osimertinib alone. All dosing conditions tested in this experiment were well-tolerated, as shown by the maintenance of body weight during the study (FIG. 5B).

Pharmacokinetic analysis (Table 2) demonstrated that 24 hours after the final dose of osimertinib, which was approximately 6˜8 hours post the final dose of formula (10b), osimertinib concentration in the plasma was below the quantitation limit of the LC/MS-MS method (2 nM), and formula (10b) 100 mg/kg dosing rendered similar plasma concentration when administered as a single agent (14.1 μM) and in combination with osimertinib (19.3 μM).

TABLE 2
Plasma exposure at 24 hours post the final dose of osimertinib (i.e., 6-8 hours post the
final dose of formula (10b)) from the study in HCC827
xenograft model shown in FIGS. 5A-5B.

| | Formula (10b) | Osimertinib |
|---|---|---|
| Treatment group | Plasma concentration (μM) | Plasma concentration (nM) |
| Osimertinib, 5 mg/kg, QD | NA | BQL |
| Formula (10b), 100 mg/kg, QD | 14.1 ± 5.6 | N/A |
| Osimertinib, 5 mg/kg, QD + | 19.3 ± 4.1 | BQL |
| Formula (10b), 100 mg/kg, QD | | |

Data represent mean ± SD. N = 4-5 mice per group. Plasma was collected from mice with < 9% BWL. BQL, below quantitation limit. N/A, LC-MS/MS analysis not conducted.

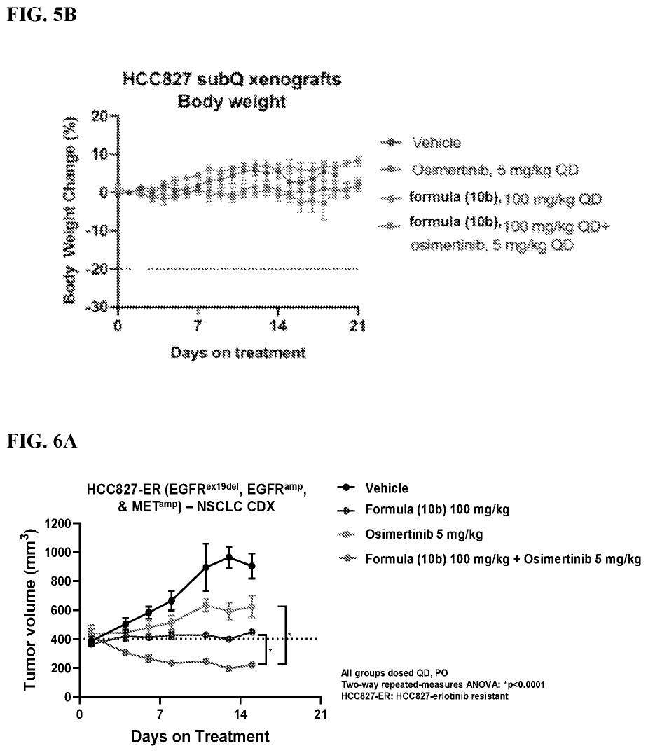

Treatment with formula (10b) as a single agent or in combination with osimertinib induces tumor growth inhibition in an EGFR^{mut} osimertinib-resistant subcutaneous model of NSCLC. A mouse tumor xenograft model was developed by subcutaneous implantation of osimertinib resistant HCC827-ER1 cells. In vitro, the HCC827-ER1 cells demonstrated 10,000-fold shift in erlotinib IC₅₀ compared with the HCC827 parental cells (unpublished data from Crown Bioscience UK Ltd). Genomic characterizations have demonstrated that the HCC827-ER1 model maintains the same EGFR activating mutation as the parental model (i.e., exon 19 deletion), does not harbor EGFR T790M mutation, and harbors c-MET amplification as a resistance mechanism, thus is anticipated to be resistant to osimertinib. The HCC827-ER1 xenograft model was used to test the anti-tumor response of formula (10b) as single-agent treatment as well as in combination with osimertinib in vivo. Mice harboring established HCC827-ER1 subcutaneous tumors (average tumor volume˜400 mm³) were treated with vehicle, osimertinib 5 mg/kg, formula (10b) 100 mg/kg or the combination of the two compounds delivered orally and each drug was administered QD for 21 days. As shown in FIG. 6A, in the osimertinib-resistant HCC827-ER1 model, tumors in mice treated with formula (10b) 100 mg/kg QD demonstrated tumor stasis, similar to what was observed with formula (10b) in the HCC827 parental model (FIG. 5A). However, tumors in mice treated with 5 mg/kg osimertinib continued to grow during the treatment period, indicating reduced osimertinib sensitivity in HCC827-ER1 model as compared to the HCC827 model. Importantly, treatment with the combination of formula (10b) and osimertinib resulted in robust tumor regression, similar to what was seen with osimertinib treatment alone in the parental HCC827 model. All dosing conditions tested in this experiment were tolerated, as demonstrated by the maintenance of body weight (£10% average BWL) during the study. Further, as shown in FIG. 6B, in the NCI-H1975 (C797S+) model, tumors in mice treated with formula (10b) alone or in combination with osimertinib resulted in robust tumor regression.

Pharmacokinetic analysis demonstrated that the plasma levels in all three treatment arms in the HCC827-ER1 xenograft model (Table 3) were similar to those observed in the HCC827 parental model (Table 1). This result demonstrates that addition of the SHP2 inhibitor formula (10b) to treatment with osimertinib leads to re-sensitization of the HCC827-ER1 model, with the combination treatment in the HCC827-ER1 model yielding a robust anti-tumor response, similar to that observed with osimertinib alone in the osimertinib-sensitive HCC827 parental model.

TABLE 3
Tumor regression and body weight changes following dosing of osimertinib
and formula (10b) in the HCC827-ER1 xenograft model shown in FIG. 6A.

| | Day 15* | | | |
|---|---|---|---|---|
| | | Mean tumor | Number of | Mean body |
| Group (n = 5) | TGI | regression | regressions | weight change |
| Vehicle | | — | 0/2 | +5.2% |
| Formula (10b), 100 mg/kg, QD | 84% | — | 0/4 | +6.3% |
| Osimertinib, 5 mg/kg, QD | 64% | — | 0/4 | +1.0% |
| Osimertinib, 5 mg/kg, QD + | — | 47% | 5/5 | −6.3% |
| Formula (10b), 100 mg/kg, QD | | | | |

*3/5 mice in the vehicle group, 1/5 mice in formula (10b) group, and 1/5 animals in the Osimertinib group were euthanized due to tumor ulcerations before day 15.

### D. Conclusions

In both the osimertinib-sensitive EGFR^{mut} HCC827 and osimertinib-resistant HCC827-ER1 xenograft models, formula (10b) 100 mg/kg QD PO robustly suppresses tumor growth, leading to tumor stasis. In the latter model, addition of the SHP2 inhibitor formula (10b) to treatment with osimertinib leads to re-sensitization of the model, compared with the osimertinib-sensitive HCC827 parental model, leading to tumor regression.

### Example 3: Pharmacokinetic and Pharmacodynamics Effect In Vivo

### A. Materials

### Test Article #1—Formula (10b):

Chemical name: 6-[(3S,4S)-4-amino-3-methyl-2-oxa-8-azaspiro[4.5]decan-8-yl]-3-(2,3-dichlorophenyl)-2,5-dimethyl-3,4-dihydropyrimidin-4-one
Molecular formula: C₂₁H₂₆Cl₂N₄O₂
Molecular weight: 437.37

### Test Article #2—Osimertinib (ChemieTek #CT-A9291):

CAS #: 1421373-65-0
Molecular weight: 499.61

Cell lines. The HCC827-ER1 (Erlotinib Resistant) cell line was derived from HCC827 cells (ATCC) at Crown Bioscience UK Ltd. The resistant cells were generated by culturing in the presence of escalating concentrations of erlotinib. The cells were maintained in RPMI-1640 medium (Sigma #RO883)+10% FBS (Sigma #F7524)+2 mM L-glutamine (Sigma #59202C)+42 μM erlotinib (LC Laboratories #E-4007). The HCC827-ER1 cells demonstrated 10,000-fold shift in erlotinib IC₅₀ in vitro compared with the HCC827 parental cells (unpublished data from Crown Bioscience UK Ltd). Genomic characterization has demonstrated that the HCC827-ER1 model maintains the same EGFR activating mutation as the parental model (i.e., exon 19 deletion), does not harbor EGFR T790M mutation, and harbors c-MET amplification as a resistance mechanism.

Test animals. Female athymic nude mice (Envigo UK, Hsd: Athymic Nude-Foxn1nu) were utilized in this experiment. Animals were between 5 and 6 weeks of age at time of xenograft implantation. Animal welfare complies with the UK Animals Scientific Procedures Act 1986 (ASPA) in line with Directive 2010/63/EU of the European Parliament and the Council of 22 Sep. 2010 on the protection of animals used for scientific purposes. All experimental data management and reporting procedures were in strict accordance with applicable Crown Bioscience UK Guidelines and Standard Operating Procedures.

All mice were housed in ISO 9001:2015 accredited animal research facilities on the Crown Bioscience UK Ltd site at Hillcrest, Dodgeford Lane, Loughborough, UK. All animals were maintained under the supervision and care of the NVS (Named Veterinary Surgeon) and NACWO (Named Animal Care and Welfare Officer) of the Animal Facilities, who oversee a comprehensive and well-executed health surveillance program. Veterinary medical services are provided on a full-time basis, and veterinarians and clinical staff are on-call for emergencies and/or special situations 24 hours a day, 7 days a week. Mice were housed in cages with a maximum of five animals and had free access to food and water with light 12-hour on/12-hour off. All animals received Teklad 2919 diet and water ad libitum. Mice were monitored daily and cages changed once every second week.

### B. Experimental Procedures

Formulation. Formula (10b) was prepared in 0.5% methylcellulose. To prepare 0.5% methylcellulose solution, methylcellulose powder (400 cP, Sigma #M0262) was added to heated 80° C. sterile water. The solution was incubated at 80° C. with stirring for 3˜4 hours, then incubated at 4° C. with continuous stirring for 18 hours. After adjusting the final volume with sterile H₂O, the solution was stirred for another 30 min at 4° C., then filtered using 0.45 μM sterile filter. The prepared 0.5% methylcellulose solution was stored at 4° C. for future use.

To prepare formula (10b) dosing suspension in 0.5% methylcellulose, the weighed compound was placed in a glass vial and 0.5% methylcellulose solution was added to the vial with a syringe. After vortexing for 30 seconds, the vial was sonicated in a water bath sonicator (Fisher Scientific Ultrasonic Bath 5.7L Model 3800, #15337418) on “High” setting at room temperature for ˜ 20 min until an off-white suspension without major white solids was obtained. The prepared dosing suspension was stored at 4° C. with gentle continuous stirring. Fresh dosing suspension was prepared once a week.

Osimertinib was prepared in 0.5% hydroxypropyl methylcellulose (HPMC). To prepare 0.5% HPMC solution, 0.5 g of HPMC powder (Sigma #H9262) was added to approximately 80 mL of sterile water in a glass beaker, and mixed on a magnetic stir plate for ˜ 1 hour. Once all powder dissolved, the solution was transferred to a 100 mL volumetric flask. The beaker was rinsed with sterile water and the final volume was adjusted to 100 mL using the rinse. The prepared 0.5% HPMC solution was stored at 4° C. for future use.

To prepare osimertinib dosing suspension in 0.5% HPMC, the weighed compound was placed in a glass vial and prepared 0.5% HPMC was added. The mixture was left on a magnetic stir plate overnight at room temperature to ensure all particles were well suspended. The prepared dosing suspension was stored at 4° C. with gentle continuous stirring. Fresh dosing suspension was prepared once a week.

In vivo modeling, treatment and data analysis. For xenograft studies with the HCC827-ER1 cells, 5 million cells in 200 μL Matrigel (Corning #354234) were injected subcutaneously into the left flank of 5˜6 weeks old female athymic nude mice (Envigo UK). Tumor size measurement was performed with caliper and calculated using a standard formula: length×width₂/2, where length and width were the long and short diameters of the tumor, respectively. When the average tumor volume reached ˜400 mm₃, mice were randomized into groups of 12 based on both tumor volume and body weight, and treated with vehicle (0.5% methylcellulose, QD in the morning+0.5% HPMC, QD in the afternoon), osimertinib (0.5% methylcellulose, QD in the morning+osimertinib 5 mg/kg, QD in the afternoon), formula (10b) (formula (10b) 100 mg/kg, QD in the morning+0.5% HPMC, QD in the afternoon), or the combination (formula (10b) 100 mg/kg, QD in the morning+osimertinib 5 mg/kg in the afternoon), through oral gavage (PO). Dosing started ˜3 weeks after the subcutaneous implantation of the tumor cells. Dosing volume was 5 mL/kg for each compound, and was adjusted based on individual mouse weight. Dosing and sample harvesting followed the schedule below. There was a 6-hour interval between the morning dose and the afternoon dose, and tumor/plasma samples were harvested at three timepoints during the 24-hour dosing cycle.

| | |
|---|---|
| Day 1 | Day 2 |

Sample timepoint #1 (6 hr post Formula (10b)/24 hr post osimertinib

| | | |
|---|---|---|
| AM (Formula (10b)) | | Dose |
| PM (osimertinib) | Dose | Sample harvest 6 hours after AM dose |

Sample timepoint #2 (8 hr post Formula (10b)/2 hr post osimertinib)

| | |
|---|---|
| AM (Formula (10b)) | Dose |
| PM (osimertinib) | Dose |
| | Sample harvest 8 hr after AM |
| | dose |

Sample timepoint #3 (24 hr post Formula (10b)/18 hr post osimertinib

| | | |
|---|---|---|
| AM (Formula (10b)) | Dose | Sample harvest 24 hr AM dose of Day 1 |
| PM (osimertinib) | Dose | |

Blood collection for pharmacokinetic analysis. Blood from live animals for pharmacokinetic (PK) analyses was obtained via the lateral saphenous vein. The mouse was placed in a restraint tube and the hind leg immobilized in the extended position. Resting the foot of the animal on a solid surface, a sterile 25g needle (BD Microlance 3) was inserted into the saphenous vein on the top of the foot. The needle was removed and the blood collected using an EDTA coated capillary blood tube (Microvette CB 300 K2E) by capillary action to a volume of 100 μL.

The tube was inverted several times to distribute the EDTA evenly and then centrifuged at 13,000 rpm, 4° C. (VWR Micro Star 17R) for 5 minutes to generate plasma. The supernatant (plasma) was carefully transferred to a 1.5 mL tube and stored at −80° C.

LC-MS/MS quantitation of formula (10b) and osimertinib in mouse plasma. Concentrations of formula (10b) and osimertinib in mouse plasma were quantitated using a validated LC-MS/MS method. For each analysis, 25 μM of plasma sample was precipitated with 200 μL of acetonitrile containing imipramine (70 ng/mL) as an internal standard. This suspension was vortexed for 10 min and centrifuged on a benchtop centrifuge (Eppendorf 5424R) at 4,000 rpm for 10 min, after which 175 μL of the supernatant was transferred to a new tube and diluted with 125 μL of water prior to LC-MS/MS analysis.

LC-MS/MS analysis of formula (10b) or osimertinib was conducted on a Waters Acquity UPLC system coupled with a Waters Xevo TQ-S triple quadrupole mass spectrometer (MS/MS) operated at the positive mode (ESI+). The detection conditions of the mass spectrometer were as follows: capillary voltage, 3 kv; cone voltage, 50 ev; collision energy, 25 ev; source temperature, 150° C.; desolvation temperature, 400° C.; desolvation gas flow, 1000 L/hr; cone gas glow, 0.15 mL/min. Formula (10b) or osimertinib was separated using a SUPELCO Ascentis fused-core C18 column (2.7 μm, 2.1×20 mm) and detected by a multiple reaction monitoring transition (m/z 437.3>186.0 for formula (10b); m/z 500.0>385.1 for osimertinib; m/z 281.1>208.1 for imipramine). The injection volume was 5 μL. The LC mobile phase A was 0.1% acetic acidwater and B was 0.1% acetic acid-acetonitrile. The gradient was 5% B (0-0.3 min), 5-95% B (0.3-1.3 min), 95% B (1.3 to 1.7 min), 95-5% B (1.7-1.71 min), 5% B (1.71 to 2 min) and the flow rate was 0.75 mL/min. The column temperature was 40° C. Under these conditions, the retention time was 1.29 min for formula (10b), 1.27 min for osimertinib, and 1.37 min for the internal standard. The method was validated with the analytical range of 1-1000 ng/mL formula (10b) or osimertinib in untreated CD-1 mouse plasma. All data were processed using the MAssLynx v. 4.0 software.

In vivo pharmacodynamic assay—Transcript biomarker analysis. Total RNA from HCC827-ER1 xenograft tumors was isolated using RNeasy mini RNA isolation kit (Qiagen #74106). Approximately 5-10 mg of frozen tumor tissue was transferred to an Eppendorf safe-lock tube (Eppendorf #022600028) containing one scoop of RNase-free stainless-steel beads (MedSupply Partners #NA-SSB16-RNA) on dry ice. Tubes were moved to regular ice and 350 μL RLT lysis buffer (Qiagen #79216) with 1% 2-mercaptoethanol (Sigma #M6250) was added. The tubes were then transferred to the Bullet Blender homogenizer (NextAdvance #BBX24) for 3 min at speed 8. Additional runs were added until no tissue was visible. Afterwards, RNA isolation was conducted following the standard total RNA isolation protocol in the manual of the kit. RNA was eluted using 50 μL of nuclease-free water. Isolated RNA was quantified using Nanodrop 8000 (Thermo Fisher).

Taqman qRT-PCR assay. cDNA was synthesized using a high capacity cDNA reverse transcription kit (Thermo Fisher #4368813) with 1 μg total RNA per reaction on a BioRad Tetrad2 Thermo cycler (25° C. for 10 min, 37° C. for 120 min, 85° C. for 5 min then hold at 4° C.). The cDNA was further diluted 1:10 using ddH2O. Quantitative real-time PCR was conducted using the following Taqman probes and Taqman universal master mix II without UNG (Thermo Fisher #4440040) on QuantStudio 6 in 384-well format (Thermo Fisher), following the vendor's protocols. Each well contains a target probe of interest conjugated to FAM dye (DUSP6, Thermo Fisher Assay ID Hs01044001_m1), and an endogenous control conjugated to VIC dye (human RPLPO, Thermo Fisher #4326314E). Each cDNA was run in three technical replicates.

The ΔΔCt method was used to analyze the qRT-PCR data. Relative mRNA levels were calculated using the formula below. To calculate fold change, the relative mRNA levels of each sample were normalized to the average of vehicle-treated tumors harvested at the same timepoint (average of vehicle treated tumors was set at “1”). Calculations were done in Microsoft Excel and the graphs were generated in GraphPad Prism 8.0.

$$Relative mRNA levels = 2 \hat{} ( {C}_{t of control gene} - {C}_{t of gene of interest} )$$

RNA sequencing assay (QuantSeq assay). RNA libraries were prepared with the QuantSeq 3′ mRNA-Seq FWD Kit (Lexogen #015), following the vendor's standard protocols. Briefly, libraries were generated with 500 ng total RNA input and 11 cycles of PCR amplification of the cDNA. Batches of up to 40 samples were multiplexed and each batch was run on NextSeq 500 (Illumina) using the High Output Kit v2 (75 cycles) (Illumina #TG-160-2005).

Sample analyses were conducted using R Bioconductor. Transcript compatibility counts were obtained with kallisto (v0.44.0) running the pseudo mode with GENCODE 23 transcript annotations. Gene counts were obtained by summing all reads that uniquely mapped, and differential expression analysis was carried out using DESeq2's default settings. The heatmap was generated in GraphPad Prism 8.0.

### C. Results

In the HCC827-ER1 subcutaneous xenograft, an EGFR^{mut} osimertinib-resistant model with c-MET amplification representing the “RTK bypass” resistance mechanism, treatment with formula (10b) in combination with osimertinib causes tumor regression (Example 2). The experiments below were designed to characterize the pharmacokinetics (PK) and pharmacodynamic effect (PD) of formula (10b) in combination with osimertinib in the same model.

To determine the PK and PD of formula (10b) in combination with osimertinib in the HCC827-ER1 model, mice harboring established HCC827-ER1 subcutaneous tumors (average tumor volume˜400 mm3) were treated with vehicle, osimertinib 5 mg/kg QD, formula (10b) 100 mg/kg QD or the combination of the two compounds delivered orally for one day. Formula (10b) was administered in the morning, and osimertinib was administered in the afternoon, with a 6-hour interval between doses. Plasma and tumor samples were harvested at three timepoints during the 24-hour dosing cycle-6 hr post formula (10b)/24 hr post osimertinib (dosing trough of osimertinib), 8 hr post formula (10b)/2 hr post osimertinib (approximate dosing max of osimertinib), and 24 hr post formula (10b)/18 hr post osimertinib (dosing trough of formula (10b)).

Formula (10b) achieves significant concentrations in the plasma when administered as a single agent or in combination with osimertinib in mice. Pharmacokinetic analysis (Table 3) demonstrated that osimertinib, when administered either as a single agent or in combination with formula (10b), achieved plasma concentrations of about 700 nM (681 nM as monotherapy and 792 nM in combination with formula (10b)) at 2 hours post a single dose treatment, which is comparable to the human Cₘₐₓ of osimertinib. Osimertinib plasma concentration was in the low nanomolar range at 18 hours after dosing and was below the quantitation limit (quantitation limit was 2 nM) at 24 hours after dosing. These osimertinib plasma concentrations were comparable to the concentrations observed at the same time point in the corresponding efficacy study (Example 2). Treatment with formula (10b) at 100 mg/kg gave rise to significant plasma concentrations when administered as a monotherapy and in combination with osimertinib, with ˜ 30-50 M at 6 hours and 8 hours after dosing, and ˜ 1 μM at 24 hours after dose (Table 4). Formula (10b) concentration in the plasma in the PK/PD study was overall similar with (within experimental variability) that from the efficacy study (Example 2). The concentrations of each compound were comparable when the mice received monotherapy or combination, suggesting no drug-drug interactions between the two compounds in mice.

TABLE 4
Plasma concentration of formula (10b) and osimertinib in mice bearing HCC827-
ER1 subcutaneous tumors at three timepoints, following treatment with formula
(10b), osimertinib or the combination of the two compounds.

| | | | | | 24 hr post (10b) | |
|---|---|---|---|---|---|---|
| | 6 hr post (10b) | | 8 hr post (10b) | | (18 hr post | |
| | (24 hr post osimertinib) | | (2 hr post osimertinib) | | osimertinib) | |
| | 10(b) | osimertinib | 10(b) | osimertinib | 10(b) | osimertinib |
| Dosing Conditions | (μM) | (nM) | (μM) | (nM) | (μM) | (nM) |
| Osimertinib 5 mg/kg | N/A | BQL | N/A | 681 ± 277 | N/A | 2.1 ± 0.14 |
| Formula (10b) 100 mg/kg | 47.4 ± 5.2 | N/A | 35.8 ± 9.1 | N/A | 1.34 ± 0.24 | N/A |
| Osimertinib 5 mg/kg + | 38.6 ± 9.8 | BQL | 33.4 ± 11.8 | 792 ± 371 | 1.06 ± 0.27 | 9.6 ± 3.7 |
| Formula (10b) | | | | | | |

HCC827-ER1 cells were implanted subcutaneously in female athymic nude mice and allowed to grow to an average volume of 400 mm³ as monitored by caliper measurements. At this point, animals were randomized and treated with vehicle, osimertinib, Formula (10b) or the combination of the two compounds QD PO for one day. Details of the dosing regimen are shown in Experimental Procedures. Tumor and plasma samples were harvested at the three timepoints shown in the table. Data represent mean ± SD. N = 4 mice/group. N/A, LC-MS/MS analysis not conducted. BQL, below quantitation limit (2 nM).

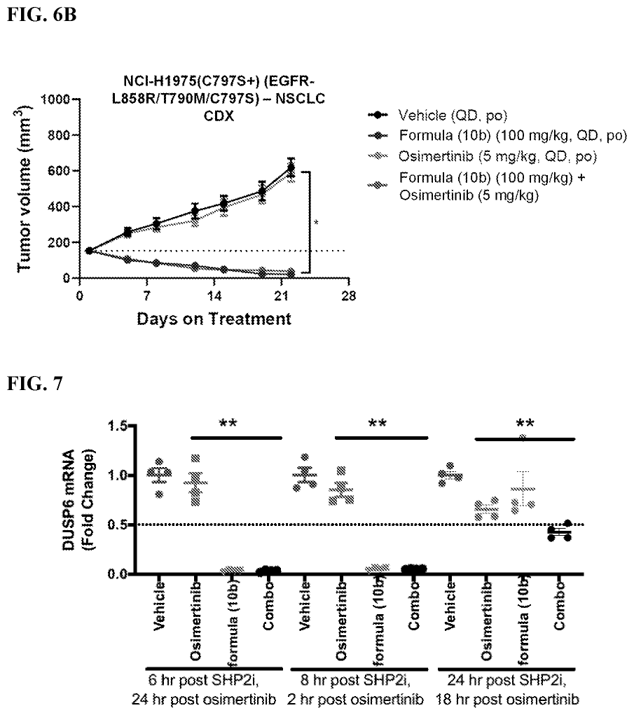

Treatment with formula (10b) as a single agent or in combination with osimertinib suppresses DUSP6 mRNA levels in HCC827-ER1 xenograft tumors. Similar to in vitro studies that evaluated the combination of osimertinib and formula (10b) in an EGFR^{mut} osimertinib-resistant model (Example 1), DUSP6 mRNA was used as a readout of MAPK pathway activity. As shown in FIG. 7, osimertinib treatment at 5 mg/kg—a dose that caused tumor stasis in the parental HCC827 xenograft model (Example 2)—had little impact on DUSP6 mRNA levels, with the best suppression observed being less than 50%, consistent with the HCC827-ER1 model being resistant to osimertinib. In contrast, treatment with formula (10b) 100 mg/kg as a single agent suppressed DUSP6 mRNA levels over 90% at 6-hour and 8-hour after dosing (the first and second timepoints in FIG. 7). Treatment with the combination of formula (10b) and osimertinib had a similar impact on DUSP6 mRNA as formula (10b) alone at these timepoints. At 24-hour after formula (10b) dosing (the third timepoint in FIG. 7), treatment with Formula (10b) moderately suppressed DUSP6 mRNA in the HCC827-ER1 tumors (<50% suppression), and treatment with the combination of Formula (10b) and osimertinib more potently suppressed DUSP6 mRNA levels (>50% suppression). The suppression of DUSP6 mRNA in the combination group was statistically greater as compared to the osimertinib monotherapy group across all three timepoints.

Together, the data show that treatment with formula (10b) as a single agent or in combination with osimertinib suppressed DUSP6 mRNA levels in the HCC827-ER1 xenograft tumor. A greater effect of the combination treatment on DUSP6 mRNA was observed at the dosing trough of Formula (10b), which was 18-hour after osimertinib treatment.

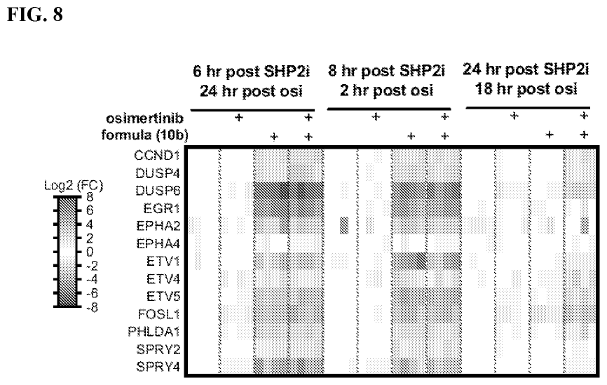

Treatment with formula (10b) as a single agent or in combination with osimertinib suppresses the MPAS-plus signature in the HCC827-ER1 xenograft tumor. In addition to DUSP6 mRNA levels, other MAPK pathway genes were examined. The MPAS (MAPK pathway activity score) signature is a signature of 10 genes that reflects MAPK pathway activity. This gene signature has been used in the clinic to evaluate the pharmacodynamic effect of the ERK inhibitor GDC-0994. Based on the MPAS signature, a 13-gene signature (“MPAS-plus”) was developed, which includes the 10 MPAS genes and three additional MAPK-targeted genes (ETV1, EGR1 and FOSL1) that are modulated by SHP2 inhibitors across multiple cell line models. Similar to what was observed for DUSP6 mRNA levels, treatment with osimertinib 5 mg/kg single agent had little effect on the MPAS-plus signature across timepoints (FIG. 8). At 6-hour and 8-hour after formula (10b) administration, treatment with formula (10b) 100 mg/kg as a single agent or in combination with osimertinib potently suppressed the mRNA levels of the MPAS-plus genes; the similar effect of formula (10b) alone and in combination with osimertinib suggests that the effect was primarily caused by formula (10b). At 24-hour after formula (10b) dosing, treatment with the combination of formula (10b) and osimertinib more potently suppressed the mRNA levels of MPAS-plus genes than either compound alone. Together, transcript analysis with a MAPK signature demonstrates that treatment with formula (10b) as a single agent or in combination with osimertinib suppressed MAPK pathway signaling in the osimertinib-resistant HCC827-ER1 tumors.

### Example 4: A Clinical Study of the SHP2 Inhibitor Compound (10b) in Combination with an EGFR Inhibitor in Patients with a Solid Tumor

A clinical study of the SHP2 inhibitor Compound (10b) in combination with an EGFR inhibitor (e.g., osimertinib or erlotinib) can be performed. Subjects of the study have a solid tumor such as non-small cell lung cancer (NSCLC), such as NSCLC characterized by an EGFR mutation. The subject may have previously completed a standard of care treatment.

The clinical study may include a dose escalation phase to evaluate the safety, tolerability, and maximum tolerated dose (MTD) and/or recommended phase 2 dose (RP2D) of Compound (10b) when used in combination with the EGFR inhibitor. Additional objectives for the dose escalation study may include assessment of preliminary antitumor activity of Compound (10b) in combination with the EGFR inhibitor (as defined by objective response rate [ORR, complete response (CR)+partial response (PR) rate], duration of response [DOR], and progression free survival [PFS] according to Response Evaluation Criteria in Solid Tumors (RECIST) v1.1, and as assessed by investigator, and overall survival [OS]); characterization of the pharmacokinetics (PK) of Compound (10b) and the EGFR inhibitor given in combination (e.g., area under the curve [AUC], maximum drug concentration [Cₘₐₓ], time to Cₘₐₓ [Tₘₐₓ], half-life) of Compound (10b) and the EGFR inhibitor from plasma or serum concentration-time data); characterization of circulating and intratumoral target engagement (pharmacodynamic activity) of Compound (10b) in combination with the EGFR inhibitor (e.g., raw, normalized, and/or baseline adjusted analyte signal in circulating and intratumoral target engagement biomarkers of Compound (10b) activity in combination with the EGFR inhibitor); and characterization of the immunogenicity of the EGFR inhibitor when given in combination with Compound (10b). Peripheral and intratumoral biomarkers may also be assessed. The dose escalation phase may include, e.g., 5-20 patients.

The clinical study may also include a dose expansion/optimization period to evaluate the antitumor activity of Compound (10b), as defined by the ORR (per investigator) according to RECIST v1.1, when used in combination with the EGFR inhibitor in subjects (e.g., subjects with advanced NSCLC with an EGFR mutation who have failed standard of care treatment). Additional objectives for the dose expansion/optimization study may include assessment of additional measures of antitumor activity of Compound (10b) in combination with the EGFR inhibitor, including ORR (per blinded independent central review [BICR]) and DOR and PFS (per investigator and BICR), as defined by RECIST v1.1, and OS; assessment of safety and tolerability of Compound (10b) at the RP2D, in combination with the EGFR inhibitor; characterization of the PK of Compound (10b) and the EGFR inhibitor given in combination; characterization of circulating and intratumoral target engagement (pharmacodynamic activity) of Compound (10b) in combination with the EGFR inhibitor; and characterization of the immunogenicity of the EGFR inhibitor when given in combination with Compound (10b). Peripheral and intratumoral biomarkers may also be assessed. The dose expansion/optimization phase may include, e.g., 10-50 patients.

Alternatively, the clinical study may include a single period including one or more dosing cohorts.

The EGFR inhibitor used in the clinical study may be, for example, gefitinib, erlotinib, afatinib, icotinib, cetuximab, panitumumab, osimertinib, vandetanib, necitumumab, brigatinib, neratinib, dacomitinib, amivantamab (JNJ-61186372), mobocertinib (TAK-788), BLU-945, varlitinib, tarloxitinib, poziotinib, and lapatinib. The EGFR inhibitor may be administered orally.

Compound (10b) is as described herein. Compound (10b) may be administered as oral capsules of, e.g., 50 and 100 mg.

Subjects in the study may have had had at least one previous line of treatment which included an EGFR inhibitor, either given as one line or in combination with another therapeutic agent. Subjects in the study may have a solid tumor, such as a solid tumor characterized by an EGFR mutation (e.g., as described herein). For examples, subjects in the study may have NSCLC with an EGFR mutation, such as EGFR positive locally advanced or metastatic NSCLC. Subjects in the study may be EGFR naïve (e.g., EGFR TKI naïve).

### Inclusion Criteria

Patients participating in the clinical study will meet the inclusion criteria listed below (as applicable):
1. Individuals ≥18 years old and be willing and able to provide signed informed consent at the Screening visit as well as comply with all study visits and requirements through the end of the study.
2. Have documentation of an EGFR mutation from local or central laboratory testing in tumor samples collected within the 1 year prior to screening.
3. Have measurable disease by RECIST v1.1.
4. Have minimum life expectancy of >12 weeks.
5. Women must have a negative serum human chorionic gonadotropin test if of childbearing potential or have had prior tubal ligation (≥1 year prior to screening), a total hysterectomy, or have had menopause (defined as 12 consecutive months of amenorrhea and confirmed by follow up hormone level assessment).
6. Patients of childbearing potential must use 2 methods of contraception for the duration of the study and for at least 5 months after the last dose of study treatment for female patients or 105 days after the last dose of study treatment for male patients, whichever is later for the individual patient. Female patients should not become pregnant or breastfeed while on this study. Female and male patients must also agree not to donate eggs (ova, oocytes) or sperm for the purpose of reproduction for at least 5 months or 105 days after the last dose of study treatment, respectively.
7. Have histologically documented, locally advanced and unresectable, or metastatic NSCLC.

### Exclusion Criteria

Patients who meet any of the exclusion criteria listed below will not be eligible for participation in the study.
1. Have participated in an interventional clinical study within the last 4 weeks OR, if applicable, be within 5 times the half-life of the investigational study drug, whichever is lesser, prior to the C1D1 visit. Patients should always adhere to other eligibility criteria that apply to specified concomitant medication regarding washout periods as specified below.
2. Have received radiotherapy or proton therapy with a limited field of radiation for palliation within 1 week of the start of study treatment, OR radiation to more than 30% of the bone marrow or with a wide field of radiation within 4 weeks of the start of study treatment.
3. Have taken any of the following:
   a. Strong or moderate inducers or inhibitors of cytochrome P450 (CYP) 3A4 or P-glycoprotein (P-gp) inducers or inhibitors (including herbal supplements or food products containing grapefruit juice, star fruit, or Seville oranges) within 14 days or 5 half-lives (whichever is longer) of Cycle 1 Day 1, and/or
   b. Drugs that are known substrates of P-gp, breast cancer resistance protein (BCRP), multidrug and toxin extrusion protein (MATE) 1 or MATE2-K, unless stopped 7 days prior to Cycle 1 Day 1 and for the study duration.
4. Have inadequate organ function as defined below:
   Hematological
      a. White blood cell count<2,000/μL
      b. Absolute neutrophil count<1,500/μL
      c. Platelets<100,000/μL
      d. Hemoglobin<9 g/dL without transfusion for ≤2 weeks or erythropoiesis-stimulating agents (e.g., Epo, Procrit) for ≤6 weeks
   Renal
      e. Serum creatinine>1.5×ULN, unless creatinine clearance≥40 mL/min (measured or calculated using the Cockcroft-Gault formula)
   Hepatic
      g. Serum total bilirubin≥1.5× institutional upper limit of normal (ULN) or ≥3.0× institutional ULN if the patient has a diagnosis of Gilbert syndrome or hemolytic anemia as confirmed by the investigator
      h. Aspartate aminotransferase/serum glutamic-oxaloacetic transaminase (AST/SGOT) and/or alanine aminotransferase/serum glutamic-pyruvic transaminase (ALT/SGPT)>2.5×ULN
   Coagulation
      i. International normalized ratio (INR) or prothrombin time (PT)>1.5×ULN unless the patient is receiving anticoagulant therapy and as long as PT or activated partial thromboplastin time (aPTT) is within the therapeutic range of intended use of anticoagulants
      j. Activated partial thromboplastin time>1.5×ULN unless the patient is receiving anticoagulant therapy and as long as PT or aPTT is within the therapeutic range of intended use of anticoagulants
5. Have active Hepatitis B infection (defined by the presence of hepatitis B surface antigen [HBsAg] or the presence of hepatitis B virus [HBV] DNA), hepatitis C infection (defined by the presence of hepatitis C virus [HCV] antibody and positive HCV RNA), or human immunodeficiency virus (HIV) infection with measurable viral load.
6. Have a life-threatening illness, medical condition, active uncontrolled infection, or organ system dysfunction (such as ascites, coagulopathy, or encephalopathy), or other reasons which, in the investigator's opinion, could compromise the participating patient's safety, or interfere with or compromise the integrity of the study outcomes.
7. Have any of the following cardiac-related issues or findings:
   a. History of significant cardiovascular disease, such as cerebrovascular accident, myocardial infarction or unstable angina, within the last 6 months before starting study treatment.
   b. Clinically significant cardiac disease, including New York Heart Association Class II or higher heart failure.
   c. History of left ventricular ejection fraction (LVEF)<50% within the previous 12 months before starting study treatment.
   d. Resting corrected QT interval (QTc)>470 msec, derived as the averaged from three electrocardiograms (ECGs), using the ECG machines provided by the sponsor for study purposes.
   e. Any clinically significant abnormalities in rhythm, conduction, or morphology of resting ECG (e.g., third degree heart block, Mobitz Type II heart block, ventricular arrhythmias, uncontrolled atrial fibrillation).
8. Have a diagnosis of another invasive malignancy within the previous 3 years other than curatively treated non-melanomatous skin cancer, superficial urothelial carcinoma, in situ cervical cancer, or any other curatively treated malignancy that is not expected to require treatment for recurrence during the course of the study.
9. Have untreated brain metastases from non-brain tumors. Patients who have had brain metastases resected or have received radiation therapy ending at least 4 weeks prior to Cycle 1, Day 1 are eligible if they meet all of the following criteria prior to first dose of study medication: a) residual neurological symptoms related to the CNS treatment Grade≤2; b) on a stable or decreasing dose of ≤10 mg daily prednisone (or equivalent) for at least 2 weeks prior to Cycle 1, Day 1, if applicable; and c) follow-up magnetic resonance imaging (MRI) within 4 weeks prior to C1D1 shows no new lesions appearing.
10. Have undergone major surgery within 4 weeks prior to study enrollment.
   Note: This does not include patients who have had procedures such as peripherally inserted central catheter line placement, thoracentesis, paracentesis, biopsies, or abscess drainage.
11. Have a history of hypersensitivity to the EGFR inhibitor or Compound (10b), active or inactive excipients of the EGFR inhibitor or Compound (10b) or drugs with a similar chemical structure or class to either the EGFR inhibitor or Compound (10b), dependent on which combination the patient could receive.
12. Have previously received a SHP2 inhibitor (eg, TNO-155, RMC-4630, RLY-1971, JAB-3068, JAB-3312 and PF-07284892).
13. Have gastrointestinal illness that, in the opinion of the investigator, would preclude absorption of Compound (10b) and/or the EGFR inhibitor (e.g., post gastrectomy, short bowel syndrome, uncontrolled Crohn's disease, celiac disease with villous atrophy, or chronic gastritis).
14. Are on dialysis.
15. Have a history of allogenic bone marrow transplant.
16. Are unable to swallow oral medications (capsules, tablets) without chewing, breaking, crushing, opening, or otherwise altering the product dosage form.
17. Have known or suspected autoimmune disease, except patients are permitted to enroll in cases of type 1 diabetes, hypothyroidism only requiring hormone replacement, skin disorders (e.g., vitiligo, psoriasis, or alopecia) not requiring systemic treatment, or conditions no expected to recur in the absence of an external trigger.
18. Have a condition requiring systemic treatment with either corticosteroids (>10 mg prednisone equivalent) or other immunosuppressive medication within 14 days of Cycle 1 Day 1. Inhaled or topical steroids, and adrenal replacement steroids >10 mg prednisone equivalent are permitted in the absence of active autoimmune disease.
19. Have received any live/attenuated vaccine within 30 days of first study treatment.

### Study Design

A study may include an initial screening period (e.g., a 30 day screening period), followed by a treatment period including multiple consecutive treatment cycles and a subsequent post-treatment follow up period. Dosing may continue for 1 or more years unless a patient is discontinued from study treatment or withdrawn from the study.

A dose escalation phase of a clinical study may follow a Bayesian optimal interval (BOIN) design. Multiple dose levels of Compound (10b) may be used in the dose escalation study, such as two or more of 250 mg, 400 mg, and 550 mg. The EGFR inhibitor will be administered in combination with Compound (10b) at appropriate dosing, such as dosing approved by the Food and Drug Administration. The dose escalation phase will be used to determine an RP2D that will be used in the dose escalation phase of the study.

In a dose escalation phase of a clinical study, subjects will receive Compound (10b) at the RP2D from the dose escalation phase in combination with the EGFR inhibitor. Depending on the results of the dose escalation phase, one or more additional cohorts including dosing of Compound (10b) at different dosing levels may be used.

Dosing of Compound (10b) or the EGFR inhibitor may be adjusted, e.g., in the event of drug-related adverse events.

FIG. 9 shows a flowchart for a trial conducted using the BOIN Design. Abbreviations: BOIN=Bayesian optimal interval design; DLT=dose limiting toxicity; MTD=maximum tolerated dose. Note: λe=19.7% and Δd=29.8%. In practice, with 6 patients/cohort, if the DLT rate is ≤1/6 then escalate the dose, if the DLT rate is ≥2/6 then de-escalate the dose.

Although the foregoing disclosure has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A method of treating cancer in a subject, comprising administering to the subject:
a) a therapeutically effective amount of a compound of formula (10b): 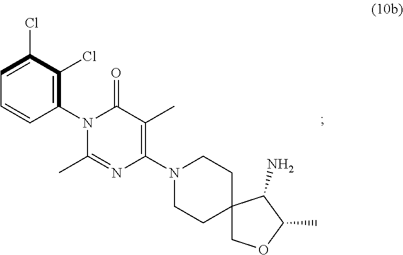
and
b) a therapeutically effective amount of an EGFR inhibitor.

2-4. (canceled)

5. The method of claim 1, wherein the subject has an EGFR mutation comprising an EGFR exon 19 deletion, exon 20 insertion, L858X mutation, T790X mutation, C797X mutation, G719X mutation, L861X mutation, S768X mutation, E709X mutation, or any combination thereof.

6. (canceled)

7. The method of claim 1, wherein the cancer is biliary tract cancer, brain cancer, breast cancer, cervical cancer, colorectal cancer, esophageal cancer, gastric cancer, head and neck squamous cell carcinoma, lung cancer, pancreatic cancer, thyroid cancer, or a combination thereof.

8. The method of claim 7, wherein the cancer is non-small cell lung cancer (NSCLC).

9. The method of claim 1, wherein the cancer is an EGFR-positive cancer resistant to an EGFR inhibitor.

10. (canceled)

11. (canceled)

12. The method of claim 1, wherein the EGFR inhibitor is a selective EGFR inhibitor or an EGFR/HER2 dual inhibitor.

13. (canceled)

14. The method of claim 1, wherein the EGFR inhibitor is erlotinib, cetuximab, panitumumab, vandetanib, afatinib, gefitinib, osimertinib, necitumumab, brigatinib, neratinib, dacomitinib, amivantamab (JNJ-61186372), mobocertinib (TAK-788), BLU-945, varlitinib, tarloxitinib, poziotinib, icotinib, or lapatinib.

15-17. (canceled)

18. The method of claim 1, wherein the subject is not previously treated with a PTPN11 inhibitor and/or an EGFR inhibitor.

19-23. (canceled)

24. The method of claim 1, wherein the compound of formula (10b) and the EGFR inhibitor are administered concomitantly.

25. (canceled)

26. The method of claim 1, wherein the compound of formula (10b) and the EGFR inhibitor are administered sequentially.

27. (canceled)

28. (canceled)

29. The method of claim 1, wherein the compound of formula (10b) and/or the EGFR inhibitor are administered orally.

30. The method of claim 1, wherein the compound of formula (10b) and the EGFR inhibitor are provided in jointly therapeutically effective amounts.

31. The method of claim 1, wherein the compound of formula (10b) and the EGFR inhibitor are provided in synergistically effective amounts.

32-44. (canceled)

45. The method of claim 1, wherein the therapeutically effective amount of the compound of formula (I) er (10b) is a total daily dosage of from about 10 mg to about 2000 mg, from about 50 mg to about 2000 mg, from about 80 mg to about 2000 mg, from about 80 mg to about 1000 mg, from about 80 mg to about 700 mg, from about 80 mg to about 550 mg, from about 80 mg to about 400 mg, from about 80 mg to about 250 mg, from about 80 mg to about 150 mg, 100 mg to about 2000 mg, from about 150 mg to about 1000 mg, from about 200 mg to about 1000 mg, from about 250 mg to about 1000 mg, from about 300 mg to about 1000 mg, from about 350 mg to about 1000 mg, from about 400 mg to about 1000 mg, from about 450 mg to about 1000 mg, from about 500 mg to about 1000 mg, from about 550 mg to about 1000 mg, from about 600 mg to about 1000 mg, from about 650 mg to about 1000 mg, from about 700 mg to about 1000 mg, from about 100 mg to about 700 mg, from about 150 mg to about 700 mg, from about 200 mg to about 700 mg, from about 250 mg to about 700 mg, from about 300 mg to about 700 mg, from about 3 mg to about 700 mg, from about 400 mg to about 700 mg, from about 450 mg to about 700 mg, from about 500 mg to about 700 mg, from about 550 mg to about 700 mg, from about 100 mg to about 550 mg, from about 150 mg to about 550 mg, from about 200 mg to about 550 mg, from about 250 mg to about 550 mg, from about 300 mg to about 550 mg, from about 350 mg to about 550 mg, from about 400 mg to about 550 mg, from about 450 mg to about 550 mg, from about 100 mg to about 400 mg, from about 150 mg to about 400 mg, from about 200 mg to about 400 mg, from about 250 mg to about 400 mg, or from about 300 mg to about 400 mg, on a salt-free and anhydrous basis.

46. (canceled)

47. The method of claim 45, wherein the therapeutically effective amount of the compound of formula (10b) is a total daily dosage of about 80 mg, about 150 mg, about 250 mg, about 400 mg, about 550 mg, or about 700 mg, on a salt-free and anhydrous basis.

48. The method of claim 1, wherein the EGFR inhibitor is osimertinib, and the therapeutically effective amount of osimertinib is a total daily dosage of about 80 mg.

49. The method of claim 1, wherein the compound of formula (10b) is administered orally and/or is administered once daily.

50. (canceled)

51. (canceled)

52. The method of claim 1, wherein administration of the compound of formula (10b) and the EGFR inhibitor reduces a volume of and/or stabilizes the cancer.

53. A pharmaceutical composition for treating cancer in a subject, comprising:
a) a therapeutically effective amount of a compound of formula (10b): 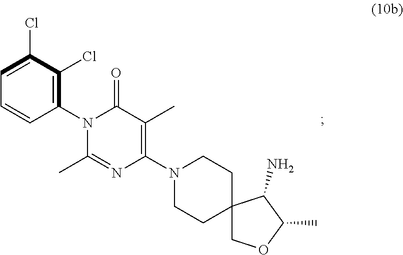
and
b) a therapeutically effective amount of an EGFR inhibitor,
together with a pharmaceutically acceptable carrier or excipient.

54-57. (canceled)

58. A kit for treating cancer in a subject, comprising:
a) a therapeutically effective amount of a compound of formula (10b): 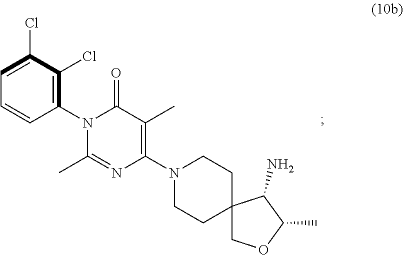
and
b) a therapeutically effective amount of an EGFR inhibitor,
together with instruction for effective administration.

59-64. (canceled)
